SEQUENCE LISTING (1 sequence)

SEQ ID NO: 1 - protein, 1255 aa, Homo sapiens [sequence omitted]

(19) United States
(12) Patent Application Publication
(10) Pub. No.: US 2024/0024320 A1
(43) Pub. Date: Jan. 25, 2024

(54) METHODS OF TREATING CANCER WITH A COMBINATION OF TUCATINIB AND AN ANTI-PD-1/ANTI-PD-L1 ANTIBODY

(71) Applicants: Seagen Inc., Bothell, WA (US); Peter MacCallum Cancer Institute, Melbourne (AU)

(72) Inventors: Scott PETERSON, Bothell, WA (US); Sherene LOI, Camberwell (AU)

(21) Appl. No.: 18/035,077

(22) PCT Filed: Nov. 16, 2021
(86) PCT No.: PCT/US2021/059534

§ 371 Date: May 2, 2023

Related U.S. Application Data
(60) Provisional application No. 63/114,797, filed on Nov. 17, 2020.

(51) Int. Cl. A61K 31/517; C07K 16/28; A61K 45/06; A61P 35/00

(52) U.S. Cl. CPC A61K 31/517; C07K 16/2818; C07K 16/2827; A61K 45/06; A61P 35/00; A61K 2039/505

(57) ABSTRACT

The disclosure provides methods of treating solid tumors with a combination of tucatinib, or salt or solvent thereof, and an anti-PD-1 antibody, or an antigen-binding fragment thereof. The disclosure also provides methods of treating solid tumors with a combination of tucatinib, or salt or solvent thereof, and an anti-PD-L1 antibody, or an antigen-binding fragment thereof.

### CROSS-REFERENCE TO RELATED APPLICATIONS

This application claims the priority benefit of U.S. Provisional Application No. 63/114,797, filed on Nov. 17, 2020, the disclosure of which is incorporated herein by reference for all purposes.

### TECHNICAL FIELD

The present invention relates to methods of treating solid tumors with a combination of tucatinib, or salt or solvate thereof, and an anti-PD-1 antibody, or an antigen-binding fragment thereof. The present invention also relates to methods of treating solid tumors with a combination of tucatinib, or salt or solvate thereof, and an anti-PD-L1 antibody, or an antigen-binding fragment thereof.

### BACKGROUND

Tucatinib ((N⁴-(4-([1,2,4]triazolo[1,5-a]pyridin-7-yloxy)-3-methylphenyl)-N-(4,4-dimethyl-4,5-dihydrooxazol-2-yl) quinazoline-4,6-diamine) (TUKYSA™; formerly known as ARRY-380 and ONT-380) is an orally (PO) administered, potent, highly selective, small-molecule tyrosine kinase inhibitor (TKI) of HER2. Tucatinib is a potent inhibitor of HER2 in vitro, and in cellular signaling assays is >1000-fold more selective for HER2 compared to the closely related kinase EGFR. The selectivity of tucatinib for HER2 reduces the potential for EGFR-related toxicities that can be seen with dual HER2/EGFR inhibitors. Tucatinib inhibits the HER2-driven mitogen-activated protein and PI3 kinase signaling pathways, resulting in inhibition of tumor cell proliferation, survival, and metastasis.

Encoded by the ERBB2 gene, human epidermal growth factor receptor 2 (HER2) is part of a family of 4 related receptor tyrosine kinases, which include HER1 (also known as epidermal growth factor receptor [EGFR]), HER2, HER3, and HER4. HER1-4 are single-pass transmembrane glycoprotein receptors containing an extracellular ligand binding region and an intracellular signaling domain. HER2 has no known ligand, but it is the preferred dimerization partner for the other HER family receptors. When overexpressed in tumors, HER2 forms ligand-independent homodimeric complexes that autophosphorylate. HER2 homo- or heterodimerization results in the activation of multiple signaling cascades, including the Ras/Raf/MEK/MAPK, PI3K/AKT, Src, and STAT pathways. These signaling pathways lead to cell proliferation, inhibition of apoptosis, and metastasis.

HER2 is a validated target in multiple solid tumors, with anti-HER2 biologics and small molecule-drugs approved for patients with HER2 overexpressing/amplified breast and gastric cancers. Amplification of the HER2 gene or overexpression of its protein occurs in approximately 15% to 20% of breast cancers.

In typical HER2+ cancers, including breast cancer, gastric cancer, and colorectal cancer, the amplification of HER2 leads to strong signal transduction through either homodimerization or heterodimerization with another ErbB-family member. This results in downstream activation of both the MAP kinase and phosphatidyl-inositol-3 (PI3) kinase pathways, which in turn enhances mitogenicity and survival.

In some cancers, however, HER2 expression is not amplified, but rather HER2 may contain an activating mutation in the kinase domain that also leads to increased signaling and mitogenicity. See WO 2018/200505. HER2 activating mutations may act as oncogenic drivers in various cancer types. See WO 2018/200505. The majority of these HER2-mutant cancers have not been associated with concurrent HER2 gene amplification, with the result that an important subgroup of HER2-altered cancers are not detected by immunohistochemistry (IHC) or in situ hybridization (ISH) methods. In the clinic, they can be identified by next generation sequencing (NGS) in either tumor biopsies or circulating cell-free DNA (cfDNA). Annals of Oncol 28:136-141 (2017). Preclinical data indicate that HER2 “hot spot” mutations may be constitutively active, have transforming capacity in vitro and in vivo and may show variable sensitivity to anti-HER2 based therapies. J Mol Diagn, 17(5):487-495 (2015), Nat Gen 51, 207-216 (2019). Recent clinical trials also revealed potential activity of HER2-targeted drugs against a variety of tumors harboring HER2 mutations. HER2-targeted agents could potentially be useful for the treatment of cancers harboring these activating mutations. ESMO Open 2017; 2: e000279. However, efforts to target cancers with HER2 mutations have met with limited clinical success, possibly because of their low frequency, inadequate understanding of the biological activity of these mutations, and difficulty in separating the drivers from the passenger mutations. The Oncologist 24(12):e1303-e1314 (2019). The role of HER2-directed therapy in these HER2-mutated cancers is the subject of active exploration.

Targeted therapy of multiple non-redundant molecular pathways regulating immune responses can enhance antitumor immunotherapy. However, not all combinations have acceptable safety and/or efficacy. There remains a need for combination therapies with an acceptable safety profile and high efficacy for the treatment of cancer, in particular for the treatment of breast cancer and cervical cancer. Targeted therapy of multiple non-redundant molecular pathways regulating immune responses can enhance antitumor immunotherapy. However, not all combinations have acceptable safety and/or efficacy. There remains a need for combination therapies with an acceptable safety profile and high efficacy for the treatment of cancer, in particular for the treatment of HER2+ solid tumors.

All references cited herein, including patent applications, patent publications, and scientific literature, are herein incorporated by reference in their entirety, as if each individual reference were specifically and individually indicated to be incorporated by reference.

### SUMMARY

Provided herein is a method of treating cancer in a subject, the method comprising administering to the subject an antibody or an antigen-binding fragment thereof, wherein the antibody binds to Programmed Death-1 (PD-1) and inhibits PD-1 activity, and tucatinib, or salt or solvate thereof, to the subject, wherein the cancer is a solid tumor. In some embodiments, the anti-PD-1 antibody or antigen-binding fragment thereof comprises the complementary determining regions (CDRs) of an antibody or antigen-binding fragment selected from the group consisting of pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof comprises the complementary determining regions (CDRs) of an antibody or antigen-binding fragment of pembrolizumab. In some embodiments, the anti-PD-1 antibody or antigen-binding fragment thereof comprises the complementary determining regions (CDRs) of an antibody or antigen-binding fragment of nivolumab. In some embodiments, the anti-PD-1 antibody or antigen-binding fragment thereof comprises the heavy chain variable region and the light chain variable region of an antibody or antigen-binding fragment selected from the group consisting of pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof comprises the heavy chain variable region and the light chain variable region of an antibody or antigen-binding fragment selected from the group consisting of pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof is selected from the group consisting of pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof is selected from the group consisting of pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof is pembrolizumab. In some embodiments, the anti-PD-1 antibody or antigen-binding fragment thereof is nivolumab. In some embodiments, the anti-PD-1 antibody or antigen-binding fragment thereof is administered intravenously. In some embodiments, one or more therapeutic effects in the subject is improved after administration of the tucatinib, or salt or solvate thereof, and the anti-PD-1 antibody or antigen-binding fragment thereof relative to a baseline. In some embodiments, the one or more therapeutic effects is selected from the group consisting of: size of a tumor derived from the cancer, objective response rate, duration of response, time to response, progression free survival, and overall survival. In some embodiments, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the tucatinib, or salt or solvate thereof, and the anti-PD-1 antibody or antigen-binding fragment thereof. In some embodiments, the objective response rate is at least about 20%, at least about 25%, at least about 30%, at least about 35%, at least about 40%, at least about 45%, at least about 50%, at least about 60%, at least about 70%, or at least about 80%. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the tucatinib, or salt or solvate thereof, and the anti-PD-1 antibody or antigen-binding fragment thereof. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the tucatinib, or salt or solvate thereof, and the anti-PD-1 antibody or antigen-binding fragment thereof. In some embodiments, the duration of response to the tucatinib, or salt or solvate thereof, and the anti-PD-1 antibody or antigen-binding fragment thereof is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the tucatinib, or salt or solvate thereof, and the anti-PD-1 antibody or antigen-binding fragment thereof. In some embodiments, infiltration of natural killer (NK) cells is increased in the solid tumor following administration of tucatinib, or salt or solvate thereof. In some embodiments, infiltration of CD8+ T cells expressing PD-1 is increased in the solid tumor following administration of tucatinib, or salt or solvate thereof. In some embodiments, infiltration of CD8+ T cells expressing IFNγ is increased in the solid tumor following administration of tucatinib, or salt or solvate thereof. In some embodiments, infiltration of CD8+ T cells expressing TIM3 is increased in the solid tumor following administration of tucatinib, or salt or solvate thereof. In some embodiments, infiltration of CD8+ T cells expressing OX40 is increased in the solid tumor following administration of tucatinib, or salt or solvate thereof. In some embodiments, infiltration of CD4+ T cells expressing FOXP3 is increased in the solid tumor following administration of tucatinib, or salt or solvate thereof. In some embodiments, infiltration of CD4+ T cells not expressing FOXP3 is increased in the solid tumor following administration of tucatinib, or salt or solvate thereof. In some embodiments, infiltration of CD4+ T cells expressing Ki67 is increased in the solid tumor following administration of tucatinib, or salt or solvate thereof. In some embodiments, the ratio of CD4+ to CD8+ T cells is increased in the solid tumor following administration of tucatinib, or salt or solvate thereof. In some embodiments, infiltration of neutrophils is decreased in the solid tumor following administration of tucatinib, or salt or solvate thereof. In some embodiments, the percentage of CD11b dendritic cells is increased in the solid tumor following administration of tucatinib, or salt or solvate thereof. In some embodiments, the percentage of MHC-II high expressing macrophages is increased in the solid tumor following administration of tucatinib, or salt or solvate thereof. In some embodiments, the percentage of MHC-II low expressing macrophages is decreased in the solid tumor following administration of tucatinib, or salt or solvate thereof. In some embodiments, the solid tumor is a HER2+ solid tumor. In some embodiments, the cancer has been determined to express a mutant form of HER2. In some embodiments, the cancer expresses a mutant form of HER2. In some embodiments, the mutant form of HER2 is determined by DNA sequencing. In some embodiments, wherein the mutant form of HER2 is determined by determining RNA sequencing. In some embodiments, the mutant form of HER2 is determined by nucleic acid sequencing. In some embodiments, the nucleic acid sequencing is next-generation sequencing (NGS). In some embodiments, the mutant form of HER2 is determined by polymerase chain reaction (PCR). In some embodiments, the mutant form of HER2 is determined by analyzing a sample obtained from the subject. In some embodiments, the sample obtained from the subject is a cell-free plasma sample. In some embodiments, the sample obtained from the subject is a tumor biopsy. In some embodiments, the HER2 mutation comprises at least one amino acid substitution, insertion, or deletion compared to the amino acid sequence of SEQ ID NO:1. In some embodiments, the HER2 mutation is an activating mutation. In some embodiments, the HER2 mutation is a mutation in the extracellular domain, the kinase domain, or the transmembrane/juxtamembrane domain, or any combination thereof. In some embodiments, the HER2 mutation is a mutation in the extracellular domain selected from the group consisting of G309A, G309E, S310F, S310Y, C311R, C311S, and C334S. In some embodiments, the HER2 mutation is a mutation in the kinase domain at an amino acid residue selected from the group consisting of Y772, G776, G778, and T798. In some embodiments, the HER2 mutation is a G776 YVMA insertion. In some embodiments, the HER2 mutation is a mutation in the kinase domain selected from the group consisting of T733I, L755P, L755S, I767M, L768S, D769N, D769Y, D769H, V777L, V777M, L841V, V842I, N857S, T862A, L869R, H878Y, and R896C. In some embodiments, the HER2 mutation is a mutation in the kinase domain at an amino acid residue V697. In some embodiments, the HER2 mutation is a mutation in the transmembrane/juxtamembrane domain selected from the group consisting of S653C, I655V, V659E, G660D, and R678Q. In some embodiments, the cancer does not have HER2 amplification, and wherein the absence of HER2 amplification is determined by immunohistochemistry (IHC). In some embodiments, the cancer has a HER2 amplification score of 0 or 1+, and wherein the HER2 amplification score is determined by immunohistochemistry (IHC). In some embodiments, the cancer has less than a 2 fold increase in HER2 protein levels. In some embodiments, the solid tumor the solid tumor has been determined to comprise HER2 overexpression/amplification. In some embodiments, the solid tumor comprises HER2 overexpression/amplification. In some embodiments, the HER2 overexpression is 3+ overexpression as determined by immunohistochemistry (IHC). In some embodiments, the HER2 amplification is determined by an in situ hybridization assay. In some embodiments, the in situ hybridization assay is fluorescence in situ hybridization (FISH) assay. In some embodiments, the in situ hybridization assay is chromogenic in situ hybridization. In some embodiments, the HER2 amplification is determined in tissue by NGS. In some embodiments, the HER2 amplification is determined in circulating tumor DNA (ctDNA) by a blood-based NGS assay. In some embodiments, the solid tumor is a metastatic solid tumor. In some embodiments, the solid tumor is locally-advanced. In some embodiments, the solid tumor is unresetable. In some embodiments, the solid tumor is selected from the group consisting of cervical cancer, uterine cancer, gallbladder cancer, cholangiocarcinoma, urothelial cancer, lung cancer, breast cancer, gastroesophageal cancer, and colorectal cancer. In some embodiments, the breast cancer is a HER2+ breast cancer. In some embodiments, the breast cancer is hormone receptor positive (HR+) breast cancer. In some embodiments, the lung cancer is non-small cell lung cancer. In some embodiments, the solid tumor is sensitive to trastuzumab. In some embodiments, the solid tumor is resistant to trastuzumab. In some embodiments, the tucatinib, or salt or solvate thereof, is administered to the subject at a dose of about 150 mg to about 650 mg. In some embodiments, the tucatinib, or salt or solvate thereof, is administered to the subject at a dose of about 300 mg. In some embodiments, the tucatinib, or salt or solvate thereof, is administered once or twice per day. In some embodiments, the tucatinib, or salt or solvate thereof, is administered to the subject at a dose of about 300 mg twice per day. In some embodiments, the tucatinib, or salt or solvate thereof, is administered to the subject orally. In some embodiments, the method further comprises administering one or more additional therapeutic agents to the subject to treat the cancer. In some embodiments, the one or more additional therapeutic agents is an anti-CTLA4 antibody or antigen-binding fragment thereof. In some embodiments, the anti-CTLA4 antibody or antigen-binding fragment thereof comprises the CDRs of ipilimumab, or a biosimilar thereof. In some embodiments, the anti-CTLA4 antibody or antigen-binding fragment thereof comprises the heavy chain variable region and the light chain variable region of ipilimumab, or a biosimilar thereof. In some embodiments, the anti-CTLA4 antibody or antigen-binding fragment thereof is ipilimumab, or a biosimilar thereof. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express CTLA4. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1. In some embodiments, treating the subject results in a tumor growth inhibition (TGI) index of at least about 85%. In some embodiments, treating the subject results in a TGI index of about 100%. In some embodiments, the subject has one or more adverse events and is further administered an additional therapeutic agent to eliminate or reduce the severity of the one or more adverse events. In some embodiments, the subject is at risk of developing one or more adverse events and is further administered an additional therapeutic agent to prevent or reduce the severity of the one or more adverse events. In some embodiments, the one or more adverse events is a grade 3 or greater adverse event. In some embodiments, the one or more adverse events is a serious adverse event. In some embodiments, the subject is a human. In some embodiments, the tucatinib, or salt or solvate thereof, is in a pharmaceutical composition comprising the tucatinib, or salt or solvate thereof, and a pharmaceutical acceptable carrier. In some embodiments, the anti-PD-1 antibody or antigen-binding fragment thereof is in a pharmaceutical composition comprising the anti-PD-1 antibody or antigen-binding fragment thereof and a pharmaceutical acceptable carrier. In some embodiments, the anti-CTLA4 antibody or antigen-binding fragment thereof is in a pharmaceutical composition comprising the anti-CTLA4 antibody or antigen-binding fragment thereof and a pharmaceutical acceptable carrier.

Also provided herein is a kit comprising tucatinib, or salt or solvate thereof, an antibody or an antigen-binding fragment thereof, wherein the antibody binds to Programmed Death-1 (PD-1) and inhibits PD-1 activity; and instructions for use of the tucatinib, or salt or solvate thereof, and the anti-PD-1 antibody or antigen-binding fragment thereof according to any of the embodiments herein.

Also provided herein is a method of treating cancer in a subject, the method comprising administering to the subject an antibody or an antigen-binding fragment thereof, wherein the antibody binds to Programmed Death Ligand-1 (PD-L1) and inhibits PD-L1 activity, and tucatinib, or salt or solvate thereof, to the subject, wherein the cancer is a solid tumor. In some embodiments, the anti-PD-L1 antibody or antigen-binding fragment thereof comprises the complementary determining regions (CDRs) of an antibody or antigen-binding fragment selected from the group consisting of atezolizumab, BMS-936559, durvalumab, avelumab, envafolimab, CK-301, CS-1001, SHR-1316, CBT-502, and BGB-A333, or a biosimilar thereof. In some embodiments, the anti-PD-L1 antibody or antigen-binding fragment thereof comprises the complementary determining regions (CDRs) of an antibody or antigen-binding fragment of atezolizumab. In some embodiments, the anti-PD-L1 antibody or antigen-binding fragment thereof comprises the complementary determining regions (CDRs) of an antibody or antigen-binding fragment of BMS936559. In some embodiments, the anti-PD-L1 antibody or antigen-binding fragment thereof comprises the complementary determining regions (CDRs) of an antibody or antigen-binding fragment of durvalumab. In some embodiments, the anti-PD-L1 antibody or antigen-binding fragment thereof comprises the complementary determining regions (CDRs) of an antibody or antigen-binding fragment of avelumab. In some embodiments, the anti-PD-L1 antibody or antigen-binding fragment thereof comprises the heavy chain variable region and the light chain variable region of an antibody or antigen-binding fragment selected from the group consisting of atezolizumab, BMS-936559, durvalumab, avelumab, envafolimab, CK-301, CS-1001, SHR-1316, CBT-502, and BGB-A333, or a biosimilar thereof. In some embodiments, the anti-PD-L1 antibody or antigen-binding fragment thereof comprises the heavy chain variable region and the light chain variable region of an antibody or antigen-binding fragment selected from the group consisting of atezolizumab, BMS-936559, durvalumab, avelumab, envafolimab, CK-301, CS-1001, SHR-1316, CBT-502, and BGB-A333. In some embodiments, the anti-PD-L1 antibody or antigen-binding fragment thereof is selected from the group consisting of atezolizumab, BMS-936559, durvalumab, avelumab, envafolimab, CK-301, CS-1001, SHR-1316, CBT-502, and BGB-A333, or a biosimilar thereof. In some embodiments, the anti-PD-L1 antibody or antigen-binding fragment thereof is selected from the group consisting of atezolizumab, BMS-936559, durvalumab, avelumab, envafolimab, CK-301, CS-1001, SHR-1316, CBT-502, and BGB-A333. In some embodiments, the anti-PD-L1 antibody or antigen-binding fragment thereof is atezolizumab. In some embodiments, the anti-PD-L1 antibody or antigen-binding fragment thereof is BMS-936559. In some embodiments, the anti-PD-L1 antibody or antigen-binding fragment thereof is durvalumab. In some embodiments, the anti-PD-L1 antibody or antigen-binding fragment thereof is avelumab. In some embodiments, the anti-PD-L1 antibody or antigen-binding fragment thereof is administered intravenously. In some embodiments, one or more therapeutic effects in the subject is improved after administration of the tucatinib, or salt or solvate thereof, and the anti-PD-L1 antibody or antigen-binding fragment thereof relative to a baseline. In some embodiments, the one or more therapeutic effects is selected from the group consisting of: size of a tumor derived from the cancer, objective response rate, duration of response, time to response, progression free survival, and overall survival. In some embodiments, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the tucatinib, or salt or solvate thereof, and the anti-PD-L1 antibody or antigen-binding fragment thereof. In some embodiments, the objective response rate is at least about 20%, at least about 25%, at least about 30%, at least about 35%, at least about 40%, at least about 45%, at least about 50%, at least about 60%, at least about 70%, or at least about 80%. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the tucatinib, or salt or solvate thereof, and the anti-PD-L1 antibody or antigen-binding fragment thereof. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the tucatinib, or salt or solvate thereof, and the anti-PD-L1 antibody or antigen-binding fragment thereof. In some embodiments, the duration of response to the tucatinib, or salt or solvate thereof, and the anti-PD-L1 antibody or antigen-binding fragment thereof is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the tucatinib, or salt or solvate thereof, and the anti-PD-L1 antibody or antigen-binding fragment thereof. In some embodiments, infiltration of natural killer (NK) cells is increased in the solid tumor following administration of tucatinib, or salt or solvate thereof. In some embodiments, infiltration of CD8+ T cells expressing PD-1 is increased in the solid tumor following administration of tucatinib, or salt or solvate thereof. In some embodiments, infiltration of CD8+ T cells expressing IFNγ is increased in the solid tumor following administration of tucatinib, or salt or solvate thereof. In some embodiments, infiltration of CD8+ T cells expressing TIM3 is increased in the solid tumor following administration of tucatinib, or salt or solvate thereof. In some embodiments, infiltration of CD8+ T cells expressing OX40 is increased in the solid tumor following administration of tucatinib, or salt or solvate thereof. In some embodiments, infiltration of CD4+ T cells expressing FOXP3 is increased in the solid tumor following administration of tucatinib, or salt or solvate thereof. In some embodiments, infiltration of CD4+ T cells not expressing FOXP3 is increased in the solid tumor following administration of tucatinib, or salt or solvate thereof. In some embodiments, infiltration of CD4+ T cells expressing Ki67 is increased in the solid tumor following administration of tucatinib, or salt or solvate thereof. In some embodiments, the ratio of CD4+ to CD8+ T cells is increased in the solid tumor following administration of tucatinib, or salt or solvate thereof. In some embodiments, infiltration of neutrophils is decreased in the solid tumor following administration of tucatinib, or salt or solvate thereof. In some embodiments, the percentage of CD11b dendritic cells is increased in the solid tumor following administration of tucatinib, or salt or solvate thereof. In some embodiments, the percentage of MHC-II high expressing macrophages is increased in the solid tumor following administration of tucatinib, or salt or solvate thereof. In some embodiments, the percentage of MHC-II low expressing macrophages is decreased in the solid tumor following administration of tucatinib, or salt or solvate thereof. In some embodiments, the solid tumor is a HER2+ solid tumor. In some embodiments, the cancer has been determined to express a mutant form of HER2. In some embodiments, the cancer expresses a mutant form of HER2. In some embodiments, the mutant form of HER2 is determined by DNA sequencing. In some embodiments, wherein the mutant form of HER2 is determined by determining RNA sequencing. In some embodiments, the mutant form of HER2 is determined by nucleic acid sequencing. In some embodiments, the nucleic acid sequencing is next-generation sequencing (NGS). In some embodiments, the mutant form of HER2 is determined by polymerase chain reaction (PCR). In some embodiments, the mutant form of HER2 is determined by analyzing a sample obtained from the subject. In some embodiments, the sample obtained from the subject is a cell-free plasma sample. In some embodiments, the sample obtained from the subject is a tumor biopsy. In some embodiments, the HER2 mutation comprises at least one amino acid substitution, insertion, or deletion compared to the amino acid sequence of SEQ ID NO:1. In some embodiments, the HER2 mutation is an activating mutation. In some embodiments, the HER2 mutation is a mutation in the extracellular domain, the kinase domain, or the transmembrane/juxtamembrane domain, or any combination thereof. In some embodiments, the HER2 mutation is a mutation in the extracellular domain selected from the group consisting of G309A, G309E, S310F, S310Y, C311R, C311S, and C334S. In some embodiments, the HER2 mutation is a mutation in the kinase domain at an amino acid residue selected from the group consisting of Y772, G776, G778, and T798. In some embodiments, the HER2 mutation is a G776 YVMA insertion. In some embodiments, the HER2 mutation is a mutation in the kinase domain selected from the group consisting of T733I, L755P, L755S, I767M, L768S, D769N, D769Y, D769H, V777L, V777M, L841V, V842I, N857S, T862A, L869R, H878Y, and R896C. In some embodiments, the HER2 mutation is a mutation in the kinase domain at an amino acid residue V697. In some embodiments, the HER2 mutation is a mutation in the transmembrane/juxtamembrane domain selected from the group consisting of S653C, I655V, V659E, G660D, and R678Q. In some embodiments, the cancer does not have HER2 amplification, and wherein the absence of HER2 amplification is determined by immunohistochemistry (IHC). In some embodiments, the cancer has a HER2 amplification score of 0 or 1+, and wherein the HER2 amplification score is determined by immunohistochemistry (IHC). In some embodiments, the cancer has less than a 2 fold increase in HER2 protein levels. In some embodiments, the solid tumor the solid tumor has been determined to comprise HER2 overexpression/amplification. In some embodiments, the solid tumor comprises HER2 overexpression/amplification. In some embodiments, the HER2 overexpression is 3+ overexpression as determined by immunohistochemistry (IHC). In some embodiments, the HER2 amplification is determined by an in situ hybridization assay. In some embodiments, the in situ hybridization assay is fluorescence in situ hybridization (FISH) assay. In some embodiments, the in situ hybridization assay is chromogenic in situ hybridization. In some embodiments, the HER2 amplification is determined in tissue by NGS. In some embodiments, the HER2 amplification is determined in circulating tumor DNA (ctDNA) by a blood-based NGS assay. In some embodiments, the solid tumor is a metastatic solid tumor. In some embodiments, the solid tumor is locally-advanced. In some embodiments, the solid tumor is unresetable. In some embodiments, the solid tumor is selected from the group consisting of cervical cancer, uterine cancer, gallbladder cancer, cholangiocarcinoma, urothelial cancer, lung cancer, breast cancer, gastroesophageal cancer, and colorectal cancer. In some embodiments, the breast cancer is a HER2+ breast cancer. In some embodiments, the breast cancer is hormone receptor positive (HR+) breast cancer. In some embodiments, the lung cancer is non-small cell lung cancer. In some embodiments, the solid tumor is sensitive to trastuzumab. In some embodiments, the solid tumor is resistant to trastuzumab. In some embodiments, the tucatinib, or salt or solvate thereof, is administered to the subject at a dose of about 150 mg to about 650 mg. In some embodiments, the tucatinib, or salt or solvate thereof, is administered to the subject at a dose of about 300 mg. In some embodiments, the tucatinib, or salt or solvate thereof, is administered once or twice per day. In some embodiments, the tucatinib, or salt or solvate thereof, is administered to the subject at a dose of about 300 mg twice per day. In some embodiments, the tucatinib, or salt or solvate thereof, is administered to the subject orally. In some embodiments, the method further comprises administering one or more additional therapeutic agents to the subject to treat the cancer. In some embodiments, the one or more additional therapeutic agents is an anti-CTLA4 antibody or antigen-binding fragment thereof. In some embodiments, the anti-CTLA4 antibody or antigen-binding fragment thereof comprises the CDRs of ipilimumab, or a biosimilar thereof. In some embodiments, the anti-CTLA4 antibody or antigen-binding fragment thereof comprises the heavy chain variable region and the light chain variable region of ipilimumab, or a biosimilar thereof. In some embodiments, the anti-CTLA4 antibody or antigen-binding fragment thereof is ipilimumab, or a biosimilar thereof. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express CTLA4. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1. In some embodiments, treating the subject results in a tumor growth inhibition (TGI) index of at least about 85%. In some embodiments, treating the subject results in a TGI index of about 100%. In some embodiments, the subject has one or more adverse events and is further administered an additional therapeutic agent to eliminate or reduce the severity of the one or more adverse events. In some embodiments, the subject is at risk of developing one or more adverse events and is further administered an additional therapeutic agent to prevent or reduce the severity of the one or more adverse events. In some embodiments, the one or more adverse events is a grade 3 or greater adverse event. In some embodiments, the one or more adverse events is a serious adverse event. In some embodiments, the subject is a human. In some embodiments, the tucatinib, or salt or solvate thereof, is in a pharmaceutical composition comprising the tucatinib, or salt or solvate thereof, and a pharmaceutical acceptable carrier. In some embodiments, the anti-PD-L1 antibody or antigen-binding fragment thereof is in a pharmaceutical composition comprising the anti-PD-L1 antibody or antigen-binding fragment thereof and a pharmaceutical acceptable carrier. In some embodiments, the anti-CTLA4 antibody or antigen-binding fragment thereof is in a pharmaceutical composition comprising the anti-CTLA4 antibody or antigen-binding fragment thereof and a pharmaceutical acceptable carrier.

Also provided herein is a kit comprising tucatinib, or salt or solvate thereof, an antibody or an antigen-binding fragment thereof, wherein the antibody binds to Programmed Death ligand-1 (PD-L1) and inhibits PD-L1 activity; and instructions for use of the tucatinib, or salt or solvate thereof, and the anti-PD-L1 antibody or antigen-binding fragment thereof according to any of the embodiments herein.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1A-1D is a series of graphs showing the effect of tucatinib on the tumor microenvironment using a trastuzumab resistant Fo5 murine tumor model.

FIG. 2A-2L is a series of graphs showing the effect of tucatinib on the tumor microenvironment using a trastuzumab sensitive H2N113 murine tumor model.

FIG. 3A-3B is a heatmap RNA profile for trastuzumab resistant Fo5 tumors that have been treated with tucatinib or vehicle control. The IFNγ-related gene signature is shown in FIG. 3A and the expanded immune signature is shown in FIG. 3B.

FIG. 4A-4B is a series of graphs showing the effect of tucatinib and various combination treatments on Fo5 tumor growth (FIG. 4A) and survival (FIG. 4B) using the treatments indicated in the legends. N=5 per treatment group.

FIG. 5A-5C shows the in vitro effect on cell growth of tucatinib at various doses in the cell lines (FIG. 5A) BT474, (FIG. 5B) SKBR3, and (FIG. 5C) H2N113.

FIG. 6 shows the H2N113 trastuzumab-sensitive HER2-positive murine tumor model scheme for assessing the effect of tucatinib on tumor size and mouse survival.

FIG. 7 shows the Fo5 trastuzumab-resistant HER2-positive murine tumor model scheme for assessing the effect of tucatinib on tumor size and mouse survival.

FIG. 8A-8B shows results of tucatinib in a H2N113 trastuzumab-sensitive HER2-positive murine tumor model. BALB/c MMTV mice inoculated with H2N113 by subcutaneous injection were treated with tucatinib or vehicle control by oral gavage at the indicated doses, and (FIG. 8A) tumor volume and (FIG. 8B) percent survival were assessed at the indicated time points. Data shown for tumor volume represent mean tumor volume (mm³)+/−SEM. P values represent two-way ANOVA analysis, post-hoc Tucky's tests for tumor growth; log ranked (Mantel-Cox) test for survival proportions. *P<0.05, **P<0.01, ***P<0.001, ****P<0.0001.

FIG. 9A-9B shows results of tucatinib in a Fo5 trastuzumab-resistant HER2-positive murine tumor model. FVB mice implanted with Fo5 tumors were treated with tucatinib or vehicle control by oral gavage at the indicated doses and (FIG. 9A) tumor volume and (FIG. 9B) percent survival were assessed at the indicated time points. Data shown for tumor volume represent mean tumor volume (mm³)+/−SEM. P values represent two-way ANOVA analysis, post-hoc Tucky's tests for tumor growth; log ranked (Mantel-Cox) test for survival proportions. *P<0.05, **P<0.01, ***P<0.001, ****P<0.0001.

FIG. 10A-10B shows validation of the Fo5 tumor model for trastuzumab resistance. FVB mice were implanted with Fo5 tumors and treated with trastuzumab or vehicle control, and then assessed for (FIG. 10A) tumor volume and (FIG. 10B) survival.

FIG. 11A-11B shows inhibition of HER2 signaling pathway after treatment with tucatinib (T) or vehicle control (V) in (FIG. 11A) Fo5 tumors and (FIG. 11B) H2N113 tumors, as assessed by Western blot.

FIG. 12A-12E shows effect on indicated cell populations at day 14 post treatment with tucatinib (T) or vehicle control (V) in H2N113 tumors as assessed by ex vivo FACS analysis, shown as frequency of parent populations.

FIG. 13A-13B shows effect on indicated cell populations at day 14 post treatment with tucatinib (T) or vehicle control (V) as assessed by ex vivo FACS analysis, shown as frequency of parent populations.

FIG. 14 shows effect on NK cell population at day 14 post treatment with tucatinib (T) or vehicle control (V) as assessed by ex vivo FACS analysis, shown as frequency of parent populations.

FIG. 15A-15B shows PD-L1 expression on CD45+ TILs after treatment with tucatinib (T) or vehicle control (V).

FIG. 16A-16D shows effect on indicated cell populations at day 10 post treatment with tucatinib (T) or vehicle control (V) in Fo5 tumors as assessed by ex vivo FACs analysis, shown as frequency of parent populations.

FIG. 17 shows results of ex vivo RNA extraction and gene expression analysis of Fo5 tumors treated with tucatinib compared to tumors treated with vehicle control. Normalized enrichment scores (NESs) for indicated hallmark genes are shown.

FIG. 18A-18B shows results of ex vivo RNA extraction and gene expression analysis of Fo5 tumors treated with tucatinib compared to tumors treated with vehicle control. NES for (A) hallmark interferon-γ and (B) hallmark interferon-α are shown.

FIG. 19 shows results of ex vivo RNA extraction and gene expression analysis of Fo5 tumors treated with tucatinib compared to tumors treated with vehicle control. NES for indicated gene ontology gene set are shown.

FIG. 20A-20B shows results of ex vivo RNA extraction and gene expression analysis of Fo5 tumors treated with tucatinib compared to tumors treated with vehicle control. NES for (FIG. 20A) gene ontology antigen binding and (FIG. 20B) gene ontology antigen processing and presentation of exogenous peptide via MHC I are shown.

FIG. 21A-21D shows heatmap RNA profiles for Fo5 tumors that have been treated with tucatinib or vehicle control. Gene signatures for the indicated genes are shown in terms of NES of tucatinib-treated Fo5 cells compared to vehicle control treated.

FIG. 22A-22B shows the effect of tucatinib treatment alone or in combination with anti-PD-L1 in H2N113 murine model as assessed by (FIG. 22A) tumor volume and (FIG. 22B) survival. Tumor volume data shown represent mean tumor volume (mm³)+/−SEM. Groups are representative of two repeats (n=4-8 per group). P values represent two-way ANOVA analysis, post-hoc Tucky's tests for tumour growth; log ranked (Mantel-Cox) test for survival proportions. *P<0.05, **P<0.01, ***P<0.001, ****P<0.0001.

FIG. 23A-23B shows the effect of tucatinib treatment alone or in combination with anti-PD-1 in Fo5 murine model as assessed by (FIG. 23A) tumor volume and (FIG. 23B) survival. Tumor volume data shown represent mean tumor volume (mm³)+/−SEM. Groups are representative of two repeats (n=4-8 per group). P values represent two-way ANOVA analysis, post-hoc Tucky's tests for tumour growth; log ranked (Mantel-Cox) test for survival proportions. *P<0.05, **P<0.01, ***P<0.001, ****P<0.0001.

FIG. 24A-24B shows the effect of tucatinib treatment alone or in combination with trastuzumab in Fo5 murine model as assessed by (FIG. 24A) tumor volume and (FIG. 24B) survival. Tumor volume data shown represent mean tumor volume (mm³)+/−SEM. Groups are representative of two repeats (n=4-8 per group). P values represent two-way ANOVA analysis, post-hoc Tucky's tests for tumour growth; log ranked (Mantel-Cox) test for survival proportions. *P<0.05, **P<0.01, ***P<0.001, ****P<0.0001.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “or” as used herein should in general be construed non-exclusively. For example, a claim to “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

The group “A or B” is typically equivalent to the group “selected from the group consisting of A and B.”

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the invention described herein include “comprising,” “consisting,” and “consisting essentially of” aspects and embodiments.

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.” The terms “about” and “approximately,” particularly in reference to a given quantity, encompass and describe the given quantity itself.

Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated.

When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11 mg/kg” is equivalent to “about 7, about 9, or about 11 mg/kg.”

The term “comprising” as used herein should in general be construed as not excluding additional ingredients. For example, a claim to “a composition comprising A” would cover compositions that include A and B; A, B, and C; A, B, C, and D; A, B, C, D, and E; and the like.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As used herein, the term “co-administering” includes sequential or simultaneous administration of two or more structurally different compounds. For example, two or more structurally different pharmaceutically active compounds can be co-administered by administering a pharmaceutical composition adapted for oral administration that contains two or more structurally different active pharmaceutically active compounds. As another example, two or more structurally different compounds can be co-administered by administering one compound and then administering the other compound. The two or more structurally different compounds can be comprised of an anti-PD-1 antibody and tucatinib. In some instances, the co-administered compounds are administered by the same route. In other instances, the co-administered compounds are administered via different routes. For example, one compound can be administered orally, and the other compound can be administered, e.g., sequentially or simultaneously, via intravenous, intramuscular, subcutaneous, or intraperitoneal injection. The simultaneously or sequentially administered compounds or compositions can be administered such that an anti-PD-1 antibody and tucatinib are simultaneously present in a subject or in a cell at an effective concentration. The simultaneously or sequentially administered compounds or compositions can be administered such that an anti-PD-L1 antibody and tucatinib are simultaneously present in a subject or in a cell at an effective concentration.

A “cancer” refers to a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor. For example, a “tumor derived from” a breast cancer refers to a tumor that is the result of a metastasized breast cancer.

In the context of cancer, the term “stage” refers to a classification of the extent of cancer. Factors that are considered when staging a cancer include but are not limited to tumor size, tumor invasion of nearby tissues, and whether the tumor has metastasized to other sites. The specific criteria and parameters for differentiating one stage from another can vary depending on the type of cancer. Cancer staging is used, for example, to assist in determining a prognosis or identifying the most appropriate treatment option(s).

One non-limiting example of a cancer staging system is referred to as the “TNM” system. In the TNM system, “T” refers to the size and extent of the main tumor, “N” refers to the number of nearby lymph nodes to which the cancer has spread, and “M” refers to whether the cancer has metastasized. “TX” denotes that the main tumor cannot be measured, “TO” denotes that the main tumor cannot be found, and “T1,” “T2,” “T3,” and “T4” denote the size or extent of the main tumor, wherein a larger number corresponds to a larger tumor or a tumor that has grown into nearby tissues. “NX” denotes that cancer in nearby lymph nodes cannot be measured, “NO” denotes that there is no cancer in nearby lymph nodes, and “N1,” “N2,” “N3,” and “N4” denote the number and location of lymph nodes to which the cancer has spread, wherein a larger number corresponds to a greater number of lymph nodes containing the cancer. “MX” denotes that metastasis cannot be measured, “MO” denotes that no metastasis has occurred, and “MI” denotes that the cancer has metastasized to other parts of the body.

As another non-limiting example of a cancer staging system, cancers are classified or graded as having one of five stages: “Stage 0,” “Stage I,” “Stage II,” “Stage III,” or “Stage IV.” Stage 0 denotes that abnormal cells are present, but have not spread to nearby tissue. This is also commonly called carcinoma in situ (CIS). CIS is not cancer, but may subsequently develop into cancer. Stages I, II, and III denote that cancer is present. Higher numbers correspond to larger tumor sizes or tumors that have spread to nearby tissues. Stage IV denotes that the cancer has metastasized. One of skill in the art will be familiar with the different cancer staging systems and readily be able to apply or interpret them.

The term “HER2” (also known as also known as HER2/neu, ERBB2, CD340, receptor tyrosine-protein kinase erbB-2, proto-oncogene Neu, and human epidermal growth factor receptor 2) refers to a member of the human epidermal growth factor receptor (HER/EGFR/ERBB) family of receptor tyrosine kinases. Amplification or overexpression of HER2 plays a significant role in the development and progression of certain aggressive types of cancer, including colorectal cancer, gastric cancer, lung cancer (e.g., non-small cell lung cancer (NSCLC)), biliary cancers (e.g., cholangiocarcinoma, gallbladder cancer), bladder cancer, esophageal cancer, melanoma, ovarian cancer, liver cancer, prostate cancer, pancreatic cancer, small intestine cancer, head and neck cancer, uterine cancer, cervical cancer, and breast cancer. Non-limiting examples of HER2 nucleotide sequences are set forth in GenBank reference numbers NP_001005862, NP_001289936, NP_001289937, NP 001289938, and NP_004448. Non-limiting examples of HER2 peptide sequences are set forth in GenBank reference numbers NP 001005862, NP_001276865, NP 001276866, NP_001276867, and NP_004439.

When HER2 is amplified or overexpressed in or on a cell, the cell is referred to as being “HER2 positive.” The level of HER2 amplification or overexpression in HER2 positive cells is commonly expressed as a score ranging from 0 to 3 (i.e., HER2 0, HER2 1+, HER2 2+, or HER2 3+), with higher scores corresponding to greater degrees of expression. Mol Biol Int. 2014:852748 (2014). The scoring method may be based on the cell membrane staining pattern as determined by immunohistochemistry and is as follows:
i. 3+: positive HER2 expression, uniform intense membrane staining of more than 30% of invasive tumor cells;
ii. 2+: equivocal for HER2 protein expression, complete membrane staining that is either nonuniform or weak in intensity but has circumferential distribution in at least 10% of cells;
iii. 0 or 1+: negative for HER2 protein expression.

The term “tucatinib,” also known as ONT-380 and ARRY-380, refers to the small molecule tyrosine kinase inhibitor that suppresses or blocks HER2 activation. Tucatinib has the following structure:

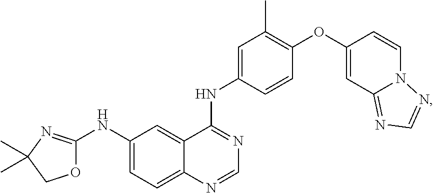 [SMILES: Cc1cc(Nc2ncnc3ccc(NC4=NC(C)(C)CO4)cc23)ccc1Oc1ccn2ncnc2c1]

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or VH) and a heavy chain constant region (C_{H} or CH). The heavy chain constant region typically is comprised of three domains, C_{H}1, C_{H}2, and C_{H}3. The heavy chains are generally inter-connected via disulfide bonds in the so-called “hinge region.” Each light chain typically is comprised of a light chain variable region (abbreviated herein as V_{L} or VL) and a light chain constant region (CL or CL). The light chain constant region typically is comprised of one domain, CL. The CL can be of κ (kappa) or λ (lambda) isotype. The terms “constant domain” and “constant region” are used interchangeably herein. An immunoglobulin can derive from any of the commonly known isotypes, including but not limited to IgA, secretory IgA, IgG, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

The term “variable region” or “variable domain” refers to the domain of an antibody heavy or light chain that is involved in binding the antibody to antigen. The variable regions of the heavy chain and light chain (V_{H} and V_{L}, respectively) of a native antibody may be further subdivided into regions of hypervariability (or hypervariable regions, which may be hypervariable in sequence and/or form of structurally defined loops), also termed complementarity-determining regions (CDRs), interspersed with regions that are more conserved, termed framework regions (FRs). The terms “complementarity determining regions” and “CDRs,” synonymous with “hypervariable regions” or “HVRs” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). Within each V_{H} and V_{L}, three CDRs and four FRs are typically arranged from amino-terminus to carboxy-terminus in the following order: FR1, CDR1, FR2, CDR2, FR3, CDR3, FR4 (See also Chothia and Lesk J. Mot. Biol., 195, 901-917 (1987)).

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half-life of significant periods of time, such as at least about 30 min, at least about 45 min, at least about one hour (h), at least about two hours, at least about four hours, at least about eight hours, at least about 12 hours (h), about 24 hours or more, about 48 hours or more, about three, four, five, six, seven or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The constant regions of the antibodies (Abs) may mediate the binding of the immunoglobulin to host tissues or factors, including various cells of the immune system (such as effector cells) and components of the complement system such as C1q, the first component in the classical pathway of complement activation. An antibody may also be a bispecific antibody, diabody, multispecific antibody or similar molecule.

The term “monoclonal antibody” as used herein refers to a preparation of antibody molecules that are recombinantly produced with a single primary amino acid sequence. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

An “isolated antibody” refers to an antibody that is substantially free of other antibodies having different antigenic specificities (e.g., an isolated antibody that binds specifically to PD-1 is substantially free of antibodies that bind specifically to antigens other than PD-1 and an isolated antibody that binds specifically to PD-L1 is substantially free of antibodies that bind specifically to antigens other than PD-L1). An isolated antibody that binds specifically to PD-1 can, however, have cross-reactivity to other antigens, such as PD-1 molecules from different species. Moreover, an isolated antibody can be substantially free of other cellular material and/or chemicals. An isolated antibody that binds specifically to PD-L1 can, however, have cross-reactivity to other antigens, such as PD-L1 molecules from different species. Moreover, an isolated antibody can be substantially free of other cellular material and/or chemicals.

A “human antibody” (HuMAb) refers to an antibody having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” and are used synonymously.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “chimeric antibody” as used herein, refers to an antibody wherein the variable region is derived from a non-human species (e.g. derived from rodents) and the constant region is derived from a different species, such as human. Chimeric antibodies may be generated by antibody engineering. “Antibody engineering” is a term used generic for different kinds of modifications of antibodies, and which is a well-known process for the skilled person. In particular, a chimeric antibody may be generated by using standard DNA techniques as described in Sambrook et al., 1989, Molecular Cloning: A laboratory Manual, New York: Cold Spring Harbor Laboratory Press, Ch. 15. Thus, the chimeric antibody may be a genetically or an enzymatically engineered recombinant antibody. It is within the knowledge of the skilled person to generate a chimeric antibody, and thus, generation of the chimeric antibody according to the present invention may be performed by other methods than described herein. Chimeric monoclonal antibodies for therapeutic applications are developed to reduce antibody immunogenicity. They may typically contain non-human (e.g. murine) variable regions, which are specific for the antigen of interest, and human constant antibody heavy and light chain domains. The terms “variable region” or “variable domains” as used in the context of chimeric antibodies, refers to a region which comprises the CDRs and framework regions of both the heavy and light chains of the immunoglobulin.

An “anti-antigen antibody” refers to an antibody that binds to the antigen. For example, an anti-PD-1 antibody is an antibody that binds to the antigen PD-1, an anti-PD-L1 antibody is an antibody that binds to the antigen PD-L1, and an anti-CTLA4 antibody is an antibody that binds to the antigen CTLA4.

An “antigen-binding portion” or antigen-binding fragment” of an antibody refers to one or more fragments of an antibody that retain the ability to bind specifically to the antigen bound by the whole antibody. Examples of antibody fragments (e.g., antigen-binding fragment) include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % sequence identity of a given amino acid sequence A to, with, or against a given amino acid sequence B (which can alternatively be phrased as a given amino acid sequence A that has or comprises a certain % sequence identity to, with, or against a given amino acid sequence B) is calculated as follows:
100 times the fraction X/Y
   where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % sequence identity of A to B will not equal the % sequence identity of B to A.

As used herein, the terms “binding”, “binds” or “specifically binds” in the context of the binding of an antibody to a pre-determined antigen typically is a binding with an affinity corresponding to a K_{D} of about 10⁻⁶ M or less, e.g. 10⁻⁷ M or less, such as about 10⁻⁸ M or less, such as about 10⁻⁹ M or less, about 1010 M or less, or about 10⁻¹¹ M or even less when determined by for instance BioLayer Interferometry (BLI) technology in a Octet HTX instrument using the antibody as the ligand and the antigen as the analyte, and wherein the antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} of binding to a non-specific antigen (e.g., BSA, casein) other than the predetermined antigen or a closely related antigen. The amount with which the K_{D} of binding is lower is dependent on the K_{D} of the antibody, so that when the K_{D} of the antibody is very low, then the amount with which the K_{D} of binding to the antigen is lower than the K_{D} of binding to a non-specific antigen may be at least 10,000-fold (that is, the antibody is highly specific).

The term “K_{D}” (M), as used herein, refers to the dissociation equilibrium constant of a particular antibody-antigen interaction. Affinity, as used herein, and K_{D} are inversely related, that is that higher affinity is intended to refer to lower K_{D}, and lower affinity is intended to refer to higher K_{D}.

“Programmed Death-1” (PD-1) refers to an immunoinhibitory receptor belonging to the CD28 family. PD-1 is expressed predominantly on previously activated T-cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. In some embodiments, hPD-1 comprises the amino acid sequence found under GenBank Accession No. U64863.

“Programmed Death Ligand-1” (PD-L1) is one of two cell surface glycoprotein ligands for PD-1 (the other being PD-L2) that downregulate T-cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. In some embodiments, hPD-L1 comprises the amino acid sequence found under GenBank Accession No. Q9NZQ7.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example for the treatment of tumors, a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, by at least about 95%, by at least about 96%, by at least about 97%, by at least about 98%, or by at least about 99% in a treated subject(s) (e.g., one or more treated subjects) relative to an untreated subject(s) (e.g., one or more untreated subjects). In some embodiments, a therapeutically effective amount of an anti-cancer agent inhibits cell growth or tumor growth by 100% in a treated subject(s) (e.g., one or more treated subjects) relative to an untreated subject(s) (e.g., one or more untreated subjects).

In other embodiments of the disclosure, tumor regression can be observed and continue for a period of at least about 20 days, at least about 30 days, at least about 40 days, at least about 50 days, or at least about 60 days. Notwithstanding these ultimate measurements of therapeutic effectiveness, evaluation of immunotherapeutic drugs must also make allowance for “immune-related response patterns”.

A therapeutically effective amount of a drug (e.g., tucatinib, or salt or solvate thereof, an anti-PD-1 antibody, an anti-PD-L1 antibody, or an anti-CTLA4 antibody) includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

As used herein, “subtherapeutic dose” means a dose of a therapeutic compound (e.g., tucatinib, or salt or solvate thereof, an anti-PD-1 antibody, an anti-PD-L1 antibody, or an anti-CTLA4 antibody) that is lower than the usual or typical dose of the therapeutic compound when administered alone for the treatment of a hyperproliferative disease (e.g., cancer).

The term “tumor growth inhibition (TGI) index” refers to a value used to represent the degree to which an agent (e.g., tucatinib, an anti-PD-1 antibody, an anti-PD-L1 antibody, an anti-CTLA4 antibody, or a combination thereof) inhibits the growth of a tumor when compared to an untreated control. The TGI index is calculated for a particular time point (e.g., a specific number of days into an experiment or clinical trial) according to the following formula:

$$TGI = 1 - ( \frac{{Volume}_{treated ( Tx Day X )} - {Volume}_{treated ( Tx Day 0 )}}{{Volume}_{control ( Tx Day X )} - {Volume}_{control ( Tx Day 0 )}} ) \times 100 \% ,$$

where “Tx Day 0” denotes the first day that treatment is administered (i.e., the first day that an experimental therapy or a control therapy (e.g., vehicle only) is administered) and “Tx Day X” denotes X number of days after Day 0. Typically, mean volumes for treated and control groups are used. As a non-limiting example, in an experiment where study day 0 corresponds to “Tx Day 0” and the TGI index is calculated on study day 28 (i.e., “Tx Day 28”), if the mean tumor volume in both groups on study day 0 is 250 mm³ and the mean tumor volumes in the experimental and control groups are 125 mm³ and 750 mm³, respectively, then the TGI index on day 28 is 125%.

As used herein, the term “synergistic” or “synergy” refers to a result that is observed when administering a combination of components or agents (e.g., a combination of tucatinib and an anti-PD-1 antibody, a combination of tucatinib and an anti-PD-L1 antibody, a combination of tucatinib and an anti-CTLA4 antibody, a combination of tucatinib, an anti-PD-1 antibody, and an anti-CTLA4 antibody, or a combination of tucatinib, an anti-PD-L1 antibody, and an anti-CTLA4 antibody) produces an effect (e.g., inhibition of tumor growth, prolongation, of survival time) that is greater than the effect that would be expected based on the additive properties or effects of the individual components. In some embodiments, synergism is determined by performing a Bliss analysis (see, e.g., Foucquier et al. Pharmacol. Res. Perspect. (2015) 3(3):e00149; hereby incorporated by reference in its entirety for all purposes). The Bliss Independence model assumes that drug effects are outcomes of probabilistic processes, and assumes that the drugs act completely independently (i.e., the drugs do not interfere with one another (e.g., the drugs have different sites of action) but each contributes to a common result). According to the Bliss Independence model, the predicted effect of a combination of two drugs is calculated using the formula:

E_{AB}=E_{A}+E_{B}−E_{A}×E_{B},

where E_{A} and E_{B} represent the effects of drugs A and B, respectively, and E_{AB} represents the effect of a combination of drugs A and B. When the observed effect of the combination is greater than the predicted effect E_{AB}, then the combination of the two drugs is considered to be synergistic. When the observed effect of the combination is equal to E_{AB}, then the effect of the combination of the two drugs is considered to be additive. Alternatively, when the observed effect of the combination is less than E_{AB}, then the combination of the two drugs is considered to be antagonistic.

The observed effect of a combination of drugs can be based on, for example, the TGI index, tumor size (e.g., volume, mass), an absolute change in tumor size (e.g., volume, mass) between two or more time points (e.g., between the first day a treatment is adminstered and a particular number of days after treatment is first administered), the rate of change of tumor size (e.g., volume, mass) between two or more time points (e.g., between the first day a treatment is adminstered and a particular number of days after treatment is first administered), or the survival time of a subject or a population of subjects. When the TGI index is taken as a measure of the observed effect of a combination of drugs, the TGI index can be determined at one or more time points. When the TGI index is determined at two or more time points, in some instances the mean or median value of the multiple TGI indices can be used as a measure of the observed effect. Furthermore, the TGI index can be determined in a single subject or a population of subjects. When the TGI index is determined in a population, the mean or median TGI index in the population (e.g., at one or more time points) can be used as a measure of the observed effect. When tumor size or the rate of tumor growth is used as a measure of the observed effect, the tumor size or rate of tumor growth can be measured in a subject or a population of subjects. In some instances, the mean or median tumor size or rate of tumor growth is determined for a subject at two or more time points, or among a population of subjects at one or more time points. When survival time is measured in a population, the mean or median survival time can be used as a measure of the observed effect.

The predicted combination effect E_{AB} can be calculated using either a single dose or multiple doses of the drugs that make up the combination (e.g., tucatinib and an anti-PD-1 antibody, tucatinib and an anti-PD-L1 antibody, or tucatinib and an anti-CTLA4 antibody). In some embodiments, the predicted combination effect E_{AB} is calculated using only a single dose of each drug A and B (e.g., tucatinib and an anti-PD-1 antibody or tucatinib and an anti-PD-L1 antibody), and the values E_{A} and E_{B} are based on the observed effect of each drug when administered as a single agent. When the values for E_{A} and E_{B} are based on the observed effects of administering drugs A and B as single agents, E_{A} and E_{B} can be based on, for example, TGI indices, tumor sizes (e.g., volume, mass) measured at one or more time points, absolute changes in tumor size (e.g., volume, mass) between two or more time points (e.g., between the first day a treatment is adminstered and a particular number of days after treatment is first administered), the rates of change of tumor sizes (e.g., volume, mass) between two or more time points (e.g., between the first day a treatment is adminstered and a particular number of days after treatment is first administered), or the survival time of a subject or a population of subjects in each treatment group.

When TGI indices are taken as a measure of the observed effects, the TGI indices can be determined at one or more time points. When TGI indices are determined at two or more time points, in some instances the mean or median values can be used as measures of the observed effects. Furthermore, the TGI indices can be determined in a single subject or a population of subjects in each treatment group. When the TGI indices are determined in populations of subjects, the mean or median TGI indices in each population (e.g., at one or more time points) can be used as measures of the observed effects. When tumor sizes or the rates of tumor growth are used as measures of the observed effects, the tumor sizes or rates of tumor growth can be measured in a subject or a population of subjects in each treatment group. In some instances, the mean or median tumor sizes or rates of tumor growth are determined for subjects at two or more time points, or among populations of subjects at one or more time points. When survival time is measured in a population, mean or median survival times can be used as measures of the observed effects.

In some embodiments, the predicted combination effect E_{AB} is calculated using a range of doses (i.e., the effects of each drug, when administered as a single agent, are observed at multiple doses and the observed effects at the multiple doses are used to determine the predicted combination effect at a specific dose). As a non-limiting example, E_{AB} can be calculated using values for E_{A} and E_{B} that are calculated according to the following formulae:

$${E}_{A} = {E}_{Amax} \times \frac{{a}^{p}}{{A}_{50}^{p} + {a}^{p}} \\ {E}_{B} = {E}_{Bmax} \times \frac{{b}^{q}}{{B}_{50}^{q} + {b}^{q}} ,$$

where E_{Amax} and E_{Bmax} are the maximum effects of drugs A and B, respectively, A₅₀ and B₅₀ are the half maximum effective doses of drugs A and B, respectively, a and b are administered doses of drugs A and B, respectively, and p and q are coefficients that are derived from the shapes of the dose-response curves for drugs A and B, respectively (see, e.g., Foucquier et al. Pharmacol. Res. Perspect. (2015) 3(3):e00149).

In some embodiments, a combination of two or more drugs is considered to be synergistic when the combination produces an observed TGI index that is greater than the predicted TGI index for the combination of drugs (e.g., when the predicted TGI index is based upon the assumption that the drugs produced a combined effect that is additive). In some instances, the combination is considered to be synergistic when the observed TGI index is at least about 1%, 2%, 3%, 4%, 5%, 6%, 7%, 8%, 9%, 10%, 110%, 2, 13%, 14%, 15%, 16%, 17%, 18%, 19%, 20%, 25%, 30%, 35%, 40%, 45%, 50%, 55%, 60%, 65%, 70%, 75%, or 80% greater than the predicted TGI index for the combination of drugs.

In some embodiments, the rate of tumor growth (e.g., the rate of change of the size (e.g., volume, mass) of the tumor) is used to determine whether a combination of drugs is synergistic (e.g., the combination of drugs is synergistic when the rate of tumor growth is slower than would be expected if the combination of drugs produced an additive effect). In other embodiments, survival time is used to determine whether a combination of drugs is synergistic (e.g., a combination of drugs is synergistic when the survival time of a subject or population of subjects is longer than would be expected if the combination of drugs produced an additive effect).

An “immune-related response pattern” refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” or “ORR” refers to the sum of complete response (CR) rate and partial response (PR) rate.

As used herein, “overall survival” or “OS” refers to the percentage of individuals in a group who are likely to be alive after a particular duration of time.

The term “weight-based dose”, as referred to herein, means that a dose administered to a subject is calculated based on the weight of the subject. For example, when a subject with 60 kg body weight requires 2.0 mg/kg of an anti-PD-1 antibody, one can calculate and use the appropriate amount of the anti-PD-1 antibody (i.e., 120 mg) for administration to said subject.

The use of the term “fixed dose” with regard to a method of the disclosure means that two or more different agents (e.g., tucatinib, or salt or solvate thereof, and an anti-PD-1 antibody) are administered to a subject in particular (fixed) ratios with each other. In some embodiments, the fixed dose is based on the amount (e.g., mg) of the tucatinib, or salt or solvate thereof, or antibody. In certain embodiments, the fixed dose is based on the concentration (e.g., mg/ml) of the tucatinib, or salt or solvate thereof, or antibody. For example, a 3:1 ratio of an anti-PD-1 antibody to tucatinib, or salt or solvate thereof, administered to a subject can mean about 240 mg of the anti-PD-1 antibody and about 80 mg of tucatinib, or salt or solvate thereof, or about 3 mg/ml of the anti-PD-1 antibody and about 1 mg/ml of the tucatinib, or salt or solvate thereof, are administered to the subject.

The use of the term “flat dose” with regard to the methods and dosages of the disclosure means a dose that is administered to a subject without regard for the weight or body surface area (BSA) of the subject. The flat dose is therefore not provided as a mg/kg dose, but rather as an absolute amount of the agent (e.g., tucatinib, or salt or solvate thereof, and/or anti-PD-1 antibody). For example, a subject with 60 kg body weight and a subject with 100 kg body weight would receive the same dose of tucatinib, or salt or solvate thereof, or antibody (e.g., 300 mg of tucatinib, or salt or solvate thereof, or e.g. 240 mg of an anti-PD-1 antibody).

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Administering” or “administration” refer to the physical introduction of a therapeutic agent to a subject, using any of the various methods and delivery systems known to those skilled in the art. Exemplary routes of administration for tucatinib, or salt or solvate thereof, and/or anti-PD-1 antibody and/or anti-PD-L1 antibody and/or anti-CTLA4 antibody include intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

The terms “baseline” or “baseline value” used interchangeably herein can refer to a measurement or characterization of a symptom before the administration of the therapy (e.g., tucatinib, or salt or solvate thereof, as described herein and/or an anti-PD-1 antibody as described herein and/or an anti-PD-L1 antibody as described herein) or at the beginning of administration of the therapy. The baseline value can be compared to a reference value in order to determine the reduction or improvement of a symptom of tucatinib, or salt or solvate thereof, and/or PD-1 and/or PD-L1 associated disease contemplated herein (e.g., solid tumor). The terms “reference” or “reference value” used interchangeably herein can refer to a measurement or characterization of a symptom after administration of the therapy (e.g., tucatinib, or salt or solvate thereof, as described herein and/or an anti-PD-1 antibody as described herein and/or an anti-PD-L1 antibody as described herein). The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

The term “monotherapy” as used herein means that tucatinib, or salt or solvate thereof, anti-PD-1 antibody, or anti-PD-L1 antibody is the only anti-cancer agent administered to the subject during the treatment cycle. Other therapeutic agents, however, can be administered to the subject. For example, anti-inflammatory agents or other agents administered to a subject with cancer to treat symptoms associated with cancer, but not the underlying cancer itself, including, for example inflammation, pain, weight loss, and general malaise, can be administered during the period of monotherapy.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity
Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically significant”
Requires inpatient hospitalization or prolongation of existing hospitalization, excluding the following: 1) routine treatment or monitoring of the underlying disease, not associated with any deterioration in condition; 2) elective or pre-planned treatment for a pre-existing condition that is unrelated to the indication under study and has not worsened since signing the informed consent; and 3) social reasons and respite care in the absence of any deterioration in the patient's general condition.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±two days, i.e., every twelve days to every sixteen days. “Once about every three weeks” can include every twenty-one days±three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Description of the Embodiments

A. Methods for Treating a Solid Tumor with Tucatinib and an Anti-PD-1 or an Anti-PD-L1 Antibody

In one aspect, the present invention provides a method for treating a solid tumor in a subject comprising administering a combination of tucatinib, or salt or solvate thereof, and an anti-PD-1 antibody, or antigen-binding fragment thereof, to the subject. In another aspect, the present invention provides a method for treating a solid tumor in a subject comprising administering a combination of tucatinib, or salt or solvate thereof, and an anti-PD-L1 antibody, or antigen-binding fragment thereof, to the subject. In some embodiments, infiltration of natural killer (NK) cells is increased in the solid tumor following administration of tucatinib, or salt or solvate thereof. In some embodiments, infiltration of CD8+ T cells expressing PD-1 is increased in the solid tumor following administration of tucatinib, or salt or solvate thereof. In some embodiments, infiltration of CD8+ T cells expressing IFNγ3 is increased in the solid tumor following administration of tucatinib, or salt or solvate thereof. In some embodiments, infiltration of CD8+ T cells expressing TIM3 is increased in the solid tumor following administration of tucatinib, or salt or solvate thereof. In some embodiments, infiltration of CD8+ T cells expressing OX40 is increased in the solid tumor following administration of tucatinib, or salt or solvate thereof. In some embodiments, infiltration of CD4+ T cells expressing FOXP3 is increased in the solid tumor following administration of tucatinib, or salt or solvate thereof. In some embodiments, infiltration of CD4+ T cells not expressing FOXP3 is increased in the solid tumor following administration of tucatinib, or salt or solvate thereof. In some embodiments, infiltration of CD4+ T cells expressing Ki67 is increased in the solid tumor following administration of tucatinib, or salt or solvate thereof. In some embodiments, the ratio of CD4+ to CD8+ T cells is increased in the solid tumor following administration of tucatinib, or salt or solvate thereof. In some embodiments, infiltration of neutrophils is decreased in the solid tumor following administration of tucatinib, or salt or solvate thereof. In some embodiments, the percentage of CD11b dendritic cells is increased in the solid tumor following administration of tucatinib, or salt or solvate thereof. In some embodiments, the percentage of MHC-II high expressing macrophages is increased in the solid tumor following administration of tucatinib, or salt or solvate thereof. In some embodiments, the percentage of MHC-II low expressing macrophages is decreased in the solid tumor following administration of tucatinib, or salt or solvate thereof. In some embodiments, the method further comprises administering one or more additional therapeutic agents to the subject to treat the cancer. In some embodiments, the one or more additional therapeutic agents is an anti-CTLA4 antibody, or antigen-binding fragment thereof, as described herein. In some embodiments, the anti-CTLA4 antibody, or antigen-binding fragment thereof, comprises the CDRs of ipilimumab, or a biosimilar thereof. In some embodiments, the anti-CTLA4 antibody, or antigen-binding fragment thereof, comprises the heavy chain variable region and the light chain variable region of ipilimumab, or a biosimilar thereof. In some embodiments, the anti-CTLA4 antibody, or antigen-binding fragment thereof, is ipilimumab, or a biosimilar thereof. In some embodiments, the solid tumor is a HER2+ solid tumor.

In some embodiments, the solid tumor has been determined to express a mutant form of HER2. In some embodiments, the solid tumor expresses a mutant form of HER2. In some embodiments, the solid tumor comprises one or more HER2 alterations. In some embodiments, the one or more HER2 alterations is a HER2 mutation. In some at least one amino acid substitution, insertion, or deletion compared to the human wild-type HER2 amino acid sequence. In some embodiments, human wild-type HER2 comprises the amino acid sequence of MELAALCRWGLLLALLPPGAASTQVCTGTDMKLRLPASPETHLDMLRHLYQGCQVVQ GNLELTYLPTNASLSFLQDIQEVQGYVLIAHNQVRQVPLQRLRIVRGTQLFEDNYALAV LDNGDPLNNTTPVTGASPGGLRELQLRSLTEILKGGVLIQRNPQLCYQDTILWKDIFHKN NQLALTLIDTNRSRACHPCSPMCKGSRCWGESSEDCQSLTRTVCAGGCARCKGPLPTDC CHEQCAAGCTGPKHSDCLACLHFNHSGICELHCPALVTYNTDTFESMPNPEGRYTFGAS CVTACPYNYLSTDVGSCTLVCPLHNQEVTAEDGTQRCEKCSKPCARVCYGLGMEHLRE VRAVTSANIQEFAGCKKIFGSLAFLPESFDGDPASNTAPLQPEQLQVFETLEEITGYLYIS AWPDSLPDLSVFQNLQVIRGRILHNGAYSLTLQGLGISWLGLRSLRELGSGLALIHHNTH LCFVHTVPWDQLFRNPHQALLHTANRPEDECVGEGLACHQLCARGHCWGPGPTQCVN CSQFLRGQECVEECRVLQGLPREYVNARHCLPCHPECQPQNGSVTCFGPEADQCVACA HYKDPPFCVARCPSGVKPDLSYMPIWKFPDEEGACQPCPINCTHSCVDLDDKGCPAEQR ASPLTSIISAVVGILLVVVLGVVFGILIKRRQQKIRKYTMRRLLQETELVEPLTPSGAMPN QAQMRILKETELRKVKVLGSGAFGTVYKGIWIPDGENVKIPVAIKVLRENTSPKANKEIL DEAYVMAGVGSPYVSRLLGICLTSTVQLVTQLMPYGCLLDHVRENRGRLGSQDLLNWC MQIAKGMSYLEDVRLVHRDLAARNVLVKSPNHVKITDFGLARLLDIDETEYHADGGKV PIKWMALESILRRRF THQSDVWSYGVTVWELMTFGAKPYDGIPAREIPDLLEKGERLPQ PPICTIDVYMIMVKCWMIDSECRPRFRELVSEFSRMARDPQRFVVIQNEDLGPASPLDST FYRSLLEDDDMGDLVDAEEYLVPQQGFFCPDPAPGAGGMVHHRHRSSSTRSGGGDLTL GLEPSEEEAPRSPLAPSEGAGSDVFDGDLGMGAAKGLQSLPTHDPSPLQRYSEDPTVPLP SETDGYVAPLTCSPQPEYVNQPDVRPQPPSPREGPLPAARPAGATLERPKTLSPGKNGVV KDVFAFGGAVENPEYLTPQGGAAPQPHPPPAFSPAFDNLYYWDQDPPERGAPPSTFKGT PTAENPEYLGLDVPV (SEQ ID NO: 1). In some embodiments, the HER2 mutation is an activating mutation. In some embodiments, the HER2 mutation results in constitutive HER2 kinase domain activation. In some embodiments, the HER2 mutation is a mutation in the extracellular domain, the kinase domain, or the transmembrane/juxtamembrane domain, or any combination thereof. In some embodiments, the HER2 mutation is a mutation in the extracellular domain. In some embodiments, the HER2 mutation is a mutation in the extracellular domain selected from the group consisting of G309A, G309E, S310F, S310Y, C311R, C311S, and C334S. In some embodiments, the mutation in the extracellular domain is G309A. In some embodiments, the mutation in the extracellular domain is G309E. In some embodiments, the mutation in the extracellular domain is S310F. In some embodiments, the mutation in the extracellular domain is S310Y. In some embodiments, the mutation in the extracellular domain is C311R. In some embodiments, the mutation in the extracellular domain is C311S. In some embodiments, the mutation in the extracellular domain is C334S. In some embodiments, the HER2 mutation is a mutation in the kinase domain. In some embodiments, the HER2 mutation is a mutation in the kinase domain at an amino acid residue selected from the group consisting of Y772, G776, G778, and T798. In some embodiments, the mutation in the kinase domain is at Y772. In some embodiments, the mutation in the kinase domain is at G776. In some embodiments, the mutation at G776 is a G776 YVMA insertion (G776 ins YVMA). The G776 ins YVMA mutant form of HER2 is a mutant in which YVMA (tyrosine, valine, methionine, alanine), which is the amino acid sequence at positions 772 to 775 of the HER2 protein, is repeated once again (also referred to as “Y772_A775dup” or “A775_G776insYVMA”). Nature. 2004 Sep. 30; 431 (7008): 525-6, and Cancer Res. 2005 Mar. 1; 65 (5): 1642-6. In some embodiments, the mutation in the kinase domain is at G778. In some embodiments, the mutation in the kinase domain is at T798. In some embodiments, the HER2 mutation is a mutation in the kinase domain selected from the group consisting of T733I, L755P, L755S, I767M, L768S, D769N, D769Y, D769H, V777L, V777M, L841V, V842I, N857S, T862A, L869R, H878Y, and R896C. In some embodiments, the mutation in the kinase domain is T733I. In some embodiments, the mutation in the kinase domain is L755P. In some embodiments, the mutation in the kinase domain is L755S. In some embodiments, the mutation in the kinase domain is I767M. In some embodiments, the mutation in the kinase domain is L768S. In some embodiments, the mutation in the kinase domain is D769N. In some embodiments, the mutation in the kinase domain is D769Y. In some embodiments, the mutation in the kinase domain is D769H. In some embodiments, the mutation in the kinase domain is V777L. In some embodiments, the mutation in the kinase domain is V777M. In some embodiments, the mutation in the kinase domain is L841V. In some embodiments, the mutation in the kinase domain is V842I. In some embodiments, the mutation in the kinase domain is N857S. In some embodiments, the mutation in the kinase domain is T862A. In some embodiments, the mutation in the kinase domain is L869R. In some embodiments, the mutation in the kinase domain is H878Y. In some embodiments, the mutation in the kinase domain is R896C. In some embodiments, the HER2 mutation is a mutation in the transmembrane/juxtamembrane domain. In some embodiments, the HER2 mutation is a mutation in the kinase domain at an amino acid residue V697. In some embodiments, the HER2 mutation is a mutation in the transmembrane/juxtamembrane domain selected from the group consisting of S653C, I655V, V659E, G660D, and R678Q. In some embodiments, the mutation in the transmembrane/juxtamembrane domain is S653C. In some embodiments, the mutation in the transmembrane/juxtamembrane domain is I655V. In some embodiments, the mutation in the transmembrane/juxtamembrane domain is V659E. In some embodiments, the mutation in the transmembrane/juxtamembrane domain is G660D. In some embodiments, the mutation in the transmembrane/juxtamembrane domain is R678Q. In some embodiments, the cancer does not have HER2 amplification. In some embodiments, the cancer has been determined to not comprise a HER2 amplification. In some embodiments, HER2 amplification is determined by IHC. In some embodiments, the cancer has a HER2 amplification score of 0, wherein the HER2 amplification score is determined by IHC. In some embodiments, the cancer has a HER2 amplification score of 1+, wherein the HER2 amplification score is determined by IHC. In some embodiments, the cancer has a HER2 amplification score of 0 or 1+, wherein the HER2 amplification score is determined by IHC. In some embodiments, HER2 is not amplified if the cancer has a score of 0 as determined by IHC. In some embodiments, HER2 is not amplified if the cancer has a score of 1+ as determined by IHC. In some embodiments, the cancer has less than a 2 fold increase in HER2 protein levels compared to non-cancerous tissue. In some embodiments, the HER2 mutation is determined by DNA sequencing. In some embodiments, the HER2 mutation is determined by RNA sequencing. In some embodiments, the HER2 mutation is determined by using next generation sequencing (NGS). In some embodiments, the HER2 mutation is determined by polymerase chain reaction (PCR).

In some embodiments, the solid tumor comprises one or more HER2 alterations. In some embodiments, the one or more HER2 alterations is a HER2 overexpression/amplification. In some embodiments, the cancer has a HER2 amplification score of 2+, wherein the HER2 amplification score is determined by immunohistochemistry (IHC). In some embodiments, the cancer has a HER2 amplification score of 3+, wherein the HER2 amplification score is determined by IHC. In some embodiments, HER2 is amplified if the cancer has a score of 2+ as determined by IHC. In some embodiments, HER2 is amplified if the cancer has a score of 3+ as determined by IHC. In some embodiments, HER2 is amplified if it is overexpressed in the cancer by at least about 5%, about 10%, about 15%, about 20%, about 25%, about 30%, about 35%, about 40%, about 45%, about 50%, about 55%, about 60%, about 70%, about 75%, about 80%, about 85%, about 90%, about 95%, about 100%, about 125%, about 150%, about 175%, about 200%, about 250%, about 300%, about 350%, about 400%, about 450%, or about 500%. In some embodiments, HER2 is amplified if it is overexpressed in the cancer by at least 50%. In some embodiments, HER2 is amplified if it is overexpressed in the cancer by at least 75%. In some embodiments, HER2 is amplified if it is overexpressed in the cancer by at least 100%. In some embodiments, HER2 is amplified if it is overexpressed in the cancer by at least 150%. In some embodiments, HER2 is amplified if it is overexpressed in the cancer by at least 200%. In some embodiments, HER2 is amplified if it is overexpressed in the cancer by at least 250%. In some embodiments, HER2 is amplified if it is overexpressed in the cancer by at least 300%. In some embodiments, HER2 is amplified if it is overexpressed in the cancer by at least 400%. In some embodiments, HER2 is amplified if it is overexpressed in the cancer by at least 500%. In some embodiments, HER2 is amplified if there is at least about a 1.5 fold, about a 2 fold, about a 3 fold, about a 4 fold, about a 5 fold, about a 10 fold, about a 15 fold, about a 20 fold, about a 25 fold, about a 30 fold, about a 40 fold, about a 50 fold, about a 60 fold, about a 70 fold, about a 80 fold, about a 90 fold, or about a 100 fold increase in HER2 protein levels in the cancer. In some embodiments, HER2 is amplified if there is at least about a 1.5 fold increase in HER2 protein levels in the cancer. In some embodiments, HER2 is amplified if there is at least about a 2 fold increase in HER2 protein levels in the cancer. In some embodiments, HER2 is amplified if there is at least about a 3 fold increase in HER2 protein levels in the cancer. In some embodiments, HER2 is amplified if there is at least about a 4 fold increase in HER2 protein levels in the cancer. In some embodiments, HER2 is amplified if there is at least about a 5 fold increase in HER2 protein levels in the cancer. In some embodiments, HER2 is amplified if there is at least about a 10 fold increase in HER2 protein levels in the cancer. In some embodiments, HER2 is amplified if there is at least about a 15 fold increase in HER2 protein levels in the cancer. In some embodiments, HER2 is amplified if there is at least about a 20 fold increase in HER2 protein levels in the cancer. In some embodiments, HER2 is amplified if there is at least about a 25 fold increase in HER2 protein levels in the cancer. In some embodiments, HER2 is amplified if there is at least about a 30 fold increase in HER2 protein levels in the cancer. In some embodiments, HER2 is amplified if there is at least about a 40 fold increase in HER2 protein levels in the cancer. In some embodiments, HER2 is amplified if there is at least about a 50 fold increase in HER2 protein levels. In some embodiments, HER2 is amplified if there is at least about a 60 fold increase in HER2 protein levels in the cancer. In some embodiments, HER2 is amplified if there is at least about a 70 fold increase in HER2 protein levels in the cancer. In some embodiments, HER2 is amplified if there is at least about an 80 fold increase in HER2 protein levels in the cancer. In some embodiments, HER2 is amplified if there is at least about a 90 fold increase in HER2 protein levels in the cancer. In some embodiments, HER2 is amplified if there is at least about a 100 fold increase in HER2 protein levels in the cancer. In some embodiments, the HER2 overexpression is 3+ overexpression as determined by immunohistochemistry (IHC). In some embodiments, the HER2 amplification is determined by an in situ hybridization assay. In some embodiments, the in situ hybridization assay is fluorescence in situ hybridization (FISH) assay. In some embodiments, the in situ hybridization assay is chromogenic in situ hybridization. In some embodiments, the HER2 amplification is determined in tissue by NGS. In some embodiments, the HER2 amplification is determined in circulating tumor DNA (ctDNA) by a blood-based NGS assay.

In some embodiments, the solid tumor is a HER2+ solid tumor. In some embodiments, the solid tumor is a metastatic solid tumor. In some embodiments, the solid tumor is locally-advanced. In some embodiments, the solid tumor is unresetable. In some embodiments, the subject has been previously treated with one or more additional therapeutic agents for the solid tumor. In some embodiments, the subject has been previously treated with one or more additional therapeutic agents for the solid tumor and did not respond to the treatment. In some embodiments, the subject has been previously treated with one or more additional therapeutic agents for the solid tumor and relapsed after the treatment. In some embodiments, the subject has been previously treated with one or more additional therapeutic agents for the solid tumor and experienced disease progression during the treatment. In some embodiments, the solid tumor is sensitive to trastuzumab. In some embodiments, the solid tumor is resistant to trastuzumab. In some embodiments, the solid tumor is selected from the group consisting of cervical cancer, uterine cancer, gallbladder cancer, cholangiocarcinoma, urothelial cancer, lung cancer, breast cancer, gastroesophageal cancer, and colorectal cancer. In some embodiments, the solid tumor is cervical cancer. In some embodiments, the solid tumor is uterine cancer. In some embodiments, the solid tumor is a biliary tract cancer, e.g., gallbladder cancer or cholangiocarcinoma. In some embodiments, the solid tumor is gallbladder cancer. In some embodiments, the solid tumor is cholangiocarcinoma. In some embodiments, the solid tumor is urothelial cancer. In some embodiments, the solid tumor is lung cancer. In some embodiments, the lung cancer is non-small cell lung cancer (NSCLC). In some embodiments, the NSCLC is non-squamous NSCLC. In some embodiments, the solid tumor is breast cancer. In some embodiments, the breast cancer is HER2+ breast cancer. In some embodiments, the breast cancer is hormone receptor positive (HR+) breast cancer. In some embodiments, the breast cancer is hormone receptor negative (HR−) breast cancer. In some embodiments, the breast cancer is triple negative breast cancer. In some embodiments, the solid tumor is gastroesophageal cancer. In some embodiments, the solid tumor is colorectal cancer. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express CTLA4.

In some embodiments, the HER2 status of a sample cell is determined. The determination can be made before treatment (i.e., administration of tucatinib) begins, during treatment, or after treatment has been completed. In some instances, determination of the HER2 status results in a decision to change therapy (e.g., adding an anti-HER2 antibody to the treatment regimen, discontinuing the use of tucatinib, discontinuing therapy altogether, or switching from another treatment method to a method of the present invention).

In some embodiments, the sample cell is a cancer cell. In some instances, the sample cell is obtained from a subject who has cancer. The sample cell can be obtained as a biopsy specimen, by surgical resection, or as a fine needle aspirate (FNA). In some embodiments, the sample cell is a circulating tumor cell (CTC).

HER2 expression can be compared to a reference cell. In some embodiments, the reference cell is a non-cancer cell obtained from the same subject as the sample cell. In other embodiments, the reference cell is a non-cancer cell obtained from a different subject or a population of subjects. In some embodiments, measuring expression of HER2 comprises, for example, determining HER2 gene copy number or amplification, nucleic acid sequencing (e.g., sequencing of genomic DNA or cDNA or RNA sequencing), measuring mRNA expression, measuring protein abundance, or a combination thereof. HER2 testing methods include immunohistochemistry (IHC), in situ hybridization, fluorescence in situ hybridization (FISH), chromogenic in situ hybridization (CISH), ELISAs, and RNA quantification (e.g., of HER2 expression) using techniques such as RT-PCR and microarray analysis.

In some embodiments, the presence or absence of a HER2 mutation is confirmed by, for example, collecting tumor tissue from a cancer patient and performing a method such as real-time quantitative PCR (qRT-PCR) or microarray analysis. In some embodiments, the tumor tissue is a formalin-fixed paraffin-embedded specimen (FFPE). In some embodiments, the presence or absence of HER2 mutation is confirmed by collecting acellular circulating tumor DNA (ctDNA) from a cancer patient and performing a method such as next generation sequencing (NGS) (J Clin Oncol 2013; 31: 1997-2003, Clin Cancer Res 2012; 18: 4910-8, J Thorac Oncol 2012; 7: 85-9, Lung Cancer 2011; 74: 139-44, Cancer Res 2005; 65: 1642-6, Cancer Sci 2006; 97: 753-9, and ESMO Open 2017; 2: e000279).

Nucleic acids used to detect HER2 mutations in any of the methods described herein include genomic DNA, RNA transcribed from genomic DNA, and cDNA generated from RNA. Nucleic acids can be derived from vertebrates, for example mammals. A nucleic acid is said to be directly derived from a particular source or “derived from” a particular source if it is a copy of a nucleic acid found in that source.

In certain embodiments, the nucleic acid comprises a copy of the nucleic acid, e.g., a copy resulting from amplification. For example, amplification to obtain the desired amount of material to detect mutations may be desirable in certain instances. The amplicon may then go through a mutation detection method, such as those described below, to determine whether the mutation is present in the amplicon.

Somatic mutations or variations can be detected by certain methods known to those skilled in the art. Such methods include, but are not limited to, DNA sequencing, primers including somatic mutation-specific nucleotide incorporation assays and somatic mutation-specific primer extension assays (e.g., somatic mutation-specific PCR, somatic mutation-specific ligation chain reaction (LCR), and gap-LCR extension assays), mutation-specific oligonucleotide hybridization assays (e.g., oligonucleotide ligation assays), cleavage protection assays in which protection from cleavage agents is used to detect fluorinated bases in nucleic acid duplexes, electrophoretic analysis comparing the mobility of variants and wild type nucleic acid molecules, denaturation-gradient gel electrophoresis (e.g., DGGE as in Myers et al. (1985) Nature 313: 495), analysis of RNase cleavage on uninched base pairs, analysis of chemical or enzymatic cleavage of heteroduplex DNA, mass spectrometry (e.g., MALDI-TOF); genetic bit analysis (GBA), 5′ nuclease assay (e.g., TaqMan™), and assays using molecular pathway labels.

Detection of variation in the target nucleic acid can be accomplished by molecular cloning and sequencing of the target nucleic acid using techniques well known in the art. Alternatively, amplification techniques such as polymerase chain reaction (PCR) can be used to amplify target nucleic acid sequences directly from genomic DNA preparations from tumor tissue. The nucleic acid sequence of the amplified sequence can then be determined and variations identified therefrom. Amplification techniques are well known in the art, for example, polymerase chain reactions are described in Saiki et al., Science 239: 487, 1988; U.S. Pat. Nos. 4,683,203 and 4,683,195.

Ligase chain reactions known in the art can also be used to amplify target nucleic acid sequences. See, e.g., Wu et al., Genomics 4: 560-569 (1989). Also, a technique known as allele-specific PCR can also be used to detect somatic mutations (e.g., substitutions). See, e.g., Ruano and Kidd (1989) Nucleic Acids Research 17: 8392; McClay et al. (2002) Analytical Biochem. 301: 200-206. In certain embodiments of this technique, the 3′ terminal nucleotides of the primers are complementary to (i.e., specifically form base pairs with) certain variations of the target nucleic acid. Mutation-specific primers are used. If no specific mutation is present, no amplification product is observed. Amplification resistance mutation systems (ARMS) can also be used to detect variations (e.g., substitutions). ARMS is described, for example, in European Patent Application Publication No. 0332435, and Newton et al., Nucleic Acids Research, 17: 7, 1989.

Other methods useful for detecting variations (e.g., substitutions) include, but are not limited to: (1) mutation-specific nucleotide incorporation assays, such as single base extension assays (see, e.g., Chen et al. (2000) Genome Res. 10: 549-557); (2) mutation-specific primer extension assays (see, e.g., Ye et al. (2001) Hum. Mut. 17: 305-316); (3) 5′ nuclease assay (see, e.g., De La Vega et al. (2002) BioTechniques 32: S48-S54 (which describes the TaqMan® assay); (4) assays using molecular pathway labels (see, e.g., Tyagi et al. (1998) Nature Biotech. 16: 49-53); (5) oligonucleotide ligation assays (see, e.g., Grossman et al. (1994) Nuc. Acids Res. 22: 4527-4534) and (6) allele-specific PCR;

Variations can also be detected by mismatch detection methods. Mismatches are hybridized nucleic acid duplexes that are not 100% complementary. Lack of total complementarity can be attributed to deletions, insertions, inversions, or substitutions. One example of a mismatch detection method is, for example, a mismatch recovery detection (MRD) assay described in Faham et al., Proc. Natl. Acad. Sci. USA 102: 14717-14722 (2005). Another example of a mismatched cutting technique is the RNase protection method described in detail in Myers et al., Science 230: 1242, 1985. For example, the methods used to detect variation may include the use of labeled riboprobes that are complementary to human wild type target nucleic acids. Riboprobes and target nucleic acids derived from tissue samples are annealed (hybridized) together and subsequently digested with the enzyme RNase A, which can detect some mismatches in the duplex RNA structure. If a mismatch is detected by RNase A, it is cleaved at the site of the mismatch. Thus, when annealed RNA preparations are separated on an electrophoretic gel matrix, if mismatches are detected and cleaved by RNase A, smaller RNA products will be observed than mRNA or full length duplex RNA for DNA and riboprobes. Riboprobes need not be the full length of the target nucleic acid, but can be part of the target nucleic acid, as long as it includes a position suspected of having a mutation.

In a similar manner, DNA probes can be used to detect mismatches, for example, via enzymatic or chemical cleavage. For example, Cotton et al., Proc. Natl. Acad. Sci. USA, 85: 4397, 1988. Alternatively, discrepancies can be detected by the transition of the electrophoretic mobility of the mismatched duplex to the matched duplex. See, e.g., Cariello, Human Genetics, 42: 726, 1988. With either riboprobes or DNA probes, target nucleic acids suspected of containing mutations can be amplified prior to hybridization. In particular, if the change is a severe rearrangement such as deletion and insertion, changes in the target nucleic acid can also be detected using Southern hybridization.

Restriction fragment length polymorphism (RFLP) probes to target nucleic acids or surrounding marker genes can be used to detect variations, for example insertions or deletions. Insertions and deletions can also be detected by cloning, sequencing and amplification of target nucleic acids. Single stranded polymorphism (SSCP) assays can also be used to detect base altering variants of the allele. SSCP can be modified for the detection of ErbB2 somatic mutations. SSCP identifies base differences due to alterations in electrophoretic shifting of single stranded PCR products. Single-stranded PCR products can be produced by heating or otherwise denaturing the double-stranded PCR product. Single-stranded nucleic acids may refold or form secondary structures that are partially dependent on the base sequence. Different electrophoretic mobility of single-stranded amplification products is related to base-sequence differences at SNP positions. Denaturation gradient gel electrophoresis (DGGE) differentiates SNP alleles based on different sequence-dependent stability and melting characteristics inherent to polymorphic DNA and corresponding differences in electrophoretic migration patterns in denaturing gradient gels.

Somatic mutations or modifications can also be detected using microarrays. Microarrays are typically a multiplex technique using a series of thousands of nucleic acid probes arranged to hybridize under high-stringency conditions, e.g., with a cDNA or cRNA sample. Probe-target hybridization is typically detected and quantified by detection of fluorophore-, silver-, or chemiluminescent-labeled targets to determine the relative abundance of nucleic acid sequences at the target. In a typical microarray, the probe is attached to a hard surface by covalent bonds to the chemical matrix (via epoxy-silane, amino-silane, lysine, polyacrylamide or the like). Hard surfaces are, for example, glass, silicon chips, or microscopic beads.

Another method for the detection of somatic mutations is based on mass spectrometry. Mass spectrometry uses the unique mass of each of the four nucleotides of DNA. Potential mutation-containing ErbB2 nucleic acids can be clearly analyzed by mass spectrometry by measuring the difference in mass of nucleic acids with somatic mutations. MALDI-TOF (matrix assisted laser desorption ionization-timeout) mass spectrometry techniques are useful for extremely accurate determination of molecular weight, such as nucleic acids containing somatic mutations. Numerous approaches to nucleic acid analysis have been developed based on mass spectrometry. Exemplary mass spectrometry-based methods also include primer extension assays, which can be used in combination with other approaches, such as traditional gel-based formats and microarrays.

Sequence-specific ribozymes (U.S. Pat. No. 5,498,531) can also be used to detect somatic mutations based on the development or loss of ribozyme cleavage sites. Perfectly matched sequences can be distinguished from mismatched sequences by nuclease cleavage digestion assays or differences in melting temperatures. If a mutation affects a restriction enzyme cleavage site, the mutation can be identified by a change in the restriction enzyme digestion pattern and a corresponding change in nucleic acid fragment length determined by gel electrophoresis.

In certain embodiments of the present disclosure, protein-based detection techniques are used to detect variant proteins encoded by genes with genetic variations as disclosed herein. Determination of the presence of variant forms of proteins can be performed by any suitable technique known in the art, for example electrophoresis (e.g., denatured or non-modified polyacrylamide gel electrophoresis, two-dimensional gel electrophoresis, capillary electrophoresis). Electrophoresis, and isoelectronic focusing, chromatography (e.g., sizing chromatography, high performance liquid chromatography (HPLC), and cation exchange HPLC), mass spectroscopy (e.g., MALDI-TOF mass spectroscopy, electrospray), ionization (ESI) mass spectroscopy, and tandem mass spectroscopy). See, e.g., Ahrer and Jungabauer (2006) J. Chromatog. B. Analyt. Technol. Biomed. Life Sci. 841: 110-122. A suitable technique can be selected based in part on the nature of the variation detected. For example, variations in which substituted amino acids result in amino acid substitutions with charges different from the original amino acids can be detected by isoelectric point electrophoresis. Isoelectric electrophoresis of a polypeptide through a gel with a pH gradient at high voltage separates the protein by its isoelectric point (pi). pH gradient gels can be compared to co-operated gels containing wild type protein. In instances where the mutation results in the generation of new proteolytic cleavage sites or the abolition of existing ones, the samples can be peptide mapped using proteolytic digestion followed by appropriate electrophoresis, chromatography, or mass spectrometry techniques. The presence of the variation can also be detected using protein sequencing techniques such as Edman degradation or certain forms of mass spectroscopy.

Methods known in the art using a combination of these techniques can also be used. For example, in HPLC-microscopy tandem mass spectrometry techniques, proteolytic digestion is performed on proteins and the resulting peptide mixtures are separated by reverse phase chromatography separation. Tandem mass spectrometry is then performed and the data collected therefrom are analyzed. In another example, unmodified gel electrophoresis is combined with MALDI mass spectroscopy

In certain embodiments, a protein can be isolated from a sample using reagents such as antibodies or peptides that specifically bind to the protein, and then further analyzed to present the genetic variation using any of the techniques disclosed above.

Alternatively, the presence of the variant protein in the sample may be directed to an antibody specific for a protein having a genetic variation, i.e., an antibody that specifically binds to a protein having a mutation but does not bind to a protein having no mutation. It can be detected by an immunoaffinity assay. Such antibodies can be produced by any suitable technique known in the art. Antibodies can be used to immunoprecipitate a particular protein from a solution sample or to immunoblot a protein separated by, for example, a polyacrylamide gel. Immunocytochemical methods can also be used to detect specific protein variants in tissues or cells. For example, immunoenzymatic assays (IEMA), including enzyme-linked immunosorbent assays (ELISA), radioimmunoassay (RIA), immunoradiometric (IRMA) and sandwich assays using monoclonal or polyclonal antibodies.

B. Tucatinib Dose and Administration

In some embodiments, a dose of tucatinib is between about 0.1 mg and 10 mg per kg of the subject's body weight (e.g., about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg per kg of the subject's body weight). In other embodiments, a dose of tucatinib is between about 10 mg and 100 mg per kg of the subject's body weight (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg per kg of the subject's body weight). In some embodiments, a dose of tucatinib is at least about 100 mg to 500 mg per kg of the subject's body weight (e.g., at least about 100, 125, 150, 175, 200, 225, 250, 275, 300, 325, 350, 375, 400, 425, 450, 475, or 500 mg per kg of the subject's body weight). In particular embodiments, a dose of tucatinib is between about 1 mg and 50 mg per kg of the subject's body weight (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg per kg of the subject's body weight). In some instances, a dose of tucatinib is about 50 mg per kg of the subject's body weight.

In some embodiments, a dose of tucatinib comprises between about 1 mg and 100 mg (e.g. about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 mg) of tucatinib. In other embodiments, a dose of tucatinib comprises between about 100 mg and 1,000 mg (e.g., about 100, 105, 110, 115, 120, 125, 130, 135, 140, 145, 150, 155, 160, 165, 170, 175, 180, 185, 190, 195, 200, 205, 210, 215, 220, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg) of tucatinib. In some embodiments, a dose of tucatinib is about 150 mg to about 650 mg. In some embodiments, a dose of tucatinib is about 300 mg. In particular embodiments, a dose of tucatinib is about 300 mg (e.g., when administered twice per day).

In some embodiments, a dose of tucatinib comprises at least about 1,000 mg to 10,000 mg (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more mg) of tucatinib.

In some embodiments, a dose of tucatinib, or salt or solvate thereof, contains a therapeutically effective amount of tucatinib, or salt or solvate thereof. In other embodiments, a dose of tucatinib, or salt or solvate thereof, contains less than a therapeutically effective amount of tucatinib, or salt or solvate thereof, (e.g., when multiple doses are given in order to achieve the desired clinical or therapeutic effect).

Tucatinib, or salt or solvate thereof, can be administered by any suitable route and mode. Suitable routes of administering small molecules of the present invention are well known in the art and may be selected by those of ordinary skill in the art. In one embodiment, tucatinib, or salt or solvate thereof, administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of tucatinib, or salt or solvate thereof, is intravenous injection or infusion. In some embodiments, the route of administration of tucatinib, or salt or solvate thereof, is intravenous infusion. In some embodiments, the route of administration of tucatinib, or salt or solvate thereof, is intravenous injection or infusion. In some embodiments, the tucatinib, or salt or solvate thereof, is intravenous infusion. In some embodiments, the route of administration of tucatinib, or salt or solvate thereof, is oral.

In one embodiment of the methods or uses or product for uses provided herein, tucatinib, or salt or solvate thereof, is administered to the subject daily, twice daily, three times daily or four times daily. In some embodiments, tucatinib, or salt or solvate thereof, is administered to the subject every other day, once about every week or once about every three weeks. In some embodiments, tucatinib, or salt or solvate thereof, is administered to the subject once per day. In some embodiments, tucatinib, or salt or solvate thereof, is administered to the subject twice per day. In some embodiments, tucatinib, or salt or solvate thereof, is administered to the subject at a dose of about 300 mg twice per day. In some embodiments, tucatinib, or salt or solvate thereof, is administered to the subject at a dose of 300 mg twice per day. In some embodiments, tucatinib, or salt or solvate thereof, is administered to the subject at a dose of about 600 mg once per day. In some embodiments, tucatinib, or salt or solvate thereof, is administered to the subject at a dose of 600 mg once per day. In some embodiments, tucatinib, or salt or solvate thereof, is administered to the subject twice per day on each day of a 21-day treatment cycle. In some embodiments, the tucatinib, or salt or solvate thereof, is administered to the subject orally.

C. Anti-PD-1 Antibody

Generally, anti-PD-1 antibodies or antigen-binding fragments thereof of the disclosure bind to PD-1, e.g., human PD-1. In some embodiments, the anti-PD-1 antibody or antigen-binding fragment thereof comprises the complementary determining regions (CDRs) of an antibody or antigen-binding fragment selected from the group consisting of pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof comprises the CDRs of an antibody or antigen-binding fragment selected from the group consisting of pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof comprises the heavy chain variable region and the light chain variable region of an antibody or antigen-binding fragment selected from the group consisting of pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof comprises the heavy chain variable region and the light chain variable region of an antibody or antigen-binding fragment selected from the group consisting of pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof is selected from the group consisting of pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof is selected from the group consisting of pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof is pembrolizumab. See U.S. Pat. Nos. 8,354,509 and 8,900,587. The antibody pembrolizumab is also known as KEYTRUDA®. In some embodiments, the anti-PD-1 antibody or antigen-binding fragment thereof is nivolumab. See, e.g., U.S. Pat. No. 8,008,449; WO 2013/173223; WO 2006/121168. The antibody nivolumab is also known as OPDIVO®. In some embodiments, the anti-PD-1 antibody or antigen-binding fragment thereof is Amp-514. See, e.g., Naing et al., Annals of Oncology, Volume 27, Issue suppl_6, 1 Oct. 2016, 1072P. The antibody Amp-514 is also known as MEDI0680. In some embodiments, the anti-PD-1 antibody or antigen-binding fragment thereof is tislelizumab. See, e.g., U.S. Pat. No. 9,834,606. The antibody tislelizumab is also known as BGB-A317. In some embodiments, the anti-PD-1 antibody or antigen-binding fragment thereof is cemiplimab. See, e.g., Burova et al., Mol Cancer Ther. 2017 May; 16(5):861-870. The antibody cemiplimab is also known as REGN2810. In some embodiments, the anti-PD-1 antibody or antigen-binding fragment thereof is TSR-042 (readily available on the world wide web at www.ejcancer.com/article/S0959-8049(16)32902-1/pdf). In some embodiments, the anti-PD-1 antibody or antigen-binding fragment thereof is JNJ-63723283. See, e.g., Calvo et al., Journal of Clinical Oncology 36, no. 5_suppl (February 2018) 58-58. The antibody JNJ-63723283 is also known as JNJ-3283. In some embodiments, the anti-PD-1 antibody or antigen-binding fragment thereof is CBT-501. See, e.g., Patel et al., Journal for ImmunoTherapy of Cancer, 2017, 5 (Suppl 2):P242. The antibody CBT-501 is also known as genolimzumab. In some embodiments, the anti-PD-1 antibody or antigen-binding fragment thereof is PF-06801591. See, e.g., Youssef et al., Proceedings of the American Association for Cancer Research Annual Meeting 2017; Cancer Res 2017;77 (13 Suppl): Abstract. In some embodiments, the anti-PD-1 antibody or antigen-binding fragment thereof is JS-001. See, e.g., US 2016/0272708. In some embodiments, the anti-PD-1 antibody or antigen-binding fragment thereof is camrelizumab. See, e.g., U.S. Patent Publication US2016/376367; Huang et al., Clinical Cancer Research 2018 Mar. 15;24(6):1296-1304. The antibody camrelizumab is also known as SHR-1210 and INCSHR-1210. In some embodiments, the anti-PD-1 antibody or antigen-binding fragment thereof is PDR001. See, e.g., WO2017/106656; Naing et al., Journal of Clinical Oncology 34, no. 15_suppl (May 2016) 3060-3060. In some embodiments, the anti-PD-1 antibody or antigen-binding fragment thereof is BCD-100. See, e.g., WO2018/103017. In some embodiments, the anti-PD-1 antibody or antigen-binding fragment thereof is AGEN2034. See, e.g., WO2017/040790. In some embodiments, the anti-PD-1 antibody or antigen-binding fragment thereof is IBI-308. See, e.g., WO2017/024465; WO2017/133540. In some embodiments, the anti-PD-1 antibody or antigen-binding fragment thereof is BI-754091. See, e.g., U.S. Patent Publication US2017/334995; Johnson et al., Journal of Clinical Oncology 36, no. 5_suppl (February 2018) 212-212. In some embodiments, the anti-PD-1 antibody or antigen-binding fragment thereof is GLS-010. See, e.g., WO2017/025051. In some embodiments, the anti-PD-1 antibody or antigen-binding fragment thereof is LZM-009. See, e.g., U.S. Patent Publication US2017/210806. In some embodiments, the anti-PD-1 antibody or antigen-binding fragment thereof is AK-103. See, e.g., WO2017/071625; WO2017/166804; WO2018/036472. In some embodiments, the anti-PD-1 antibody or antigen-binding fragment thereof is MGA-012. See, e.g., WO2017/019846. In some embodiments, the anti-PD-1 antibody or antigen-binding fragment thereof is Sym-021. See, e.g., WO2017/055547. In some embodiments, the anti-PD-1 antibody or antigen-binding fragment thereof is CS1003. See, e.g., CN107840887.

Anti-PD-1 antibodies of the disclosure are preferably monoclonal, and may be multispecific, human, humanized or chimeric antibodies, single chain antibodies, Fab fragments, F(ab′) fragments, fragments produced by a Fab expression library, and PD-1 binding fragments of any of the above. In some embodiments, an anti-PD-1 antibody described herein binds specifically to PD-1 (e.g., human PD-1). The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the antibodies are antigen-binding fragments (e.g., human antigen-binding fragments) as described herein and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-PD-1 antibodies or antigen-binding fragments thereof are human, murine (e.g., mouse and rat), donkey, sheep, rabbit, goat, guinea pig, camelid, horse, or chicken.

The anti-PD-1 antibodies of the present disclosure may be monospecific, bispecific, trispecific or of greater multi specificity. Multispecific antibodies may be specific for different epitopes of PD-1 or may be specific for both PD-1 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Anti-PD-1 antibodies of the present disclosure may be described or specified in terms of the particular CDRs they comprise. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January;27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8;309(3):657-70, (“Aho” numbering scheme); and Martin et al., “Modeling antibody hypervariable loops: a combined algorithm,” PNAS, 1989, 86(23):9268-9272, (“AbM” numbering scheme). The boundaries of a given CDR may vary depending on the scheme used for identification. In some embodiments, a “CDR” or “complementarity determining region,” or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

In some embodiments, numbering of amino acid residues in CDR sequences of anti-PD-1 antibodies or antigen-binding fragments thereof provided herein are according to the IMGT numbering scheme as described in Lefranc, M. P. et al., Dev. Comp. Immunol., 2003, 27, 55-77.

In some embodiments, numbering of amino acid residues in CDR sequences of anti-PD-1 antibodies or antigen-binding fragments thereof provided herein are according to the Kabat numbering scheme as described in Kabat, E. A., et al (1991) Sequences of Proteins of Immunological Interest, Fifth Edition, U.S. Department of Health and Human Services, NTH Publication No. 91-3242.

In some embodiments, the anti-PD-1 antibodies of the present disclosure comprise the CDRs of the antibody nivolumab. See WO 2006/121168. In some embodiments, the CDRs of the antibody nivolumab are delineated using the Kabat numbering scheme (Kabat, E. A., et al. (1991) Sequences of Proteins of Immunological Interest, Fifth Edition, U.S. Department of Health and Human Services, NTH Publication No. 91-3242). The present disclosure encompasses an anti-PD-1 antibody or derivative thereof comprising a heavy or light chain variable domain, said variable domain comprising (a) a set of three CDRs, in which said set of CDRs are from the monoclonal antibody nivolumab, and (b) a set of four framework regions, in which said set of framework regions differs from the set of framework regions in the monoclonal antibody nivolumab, and in which said anti-PD-1 antibody or derivative thereof binds to PD-1. In certain embodiments, the anti-PD-1 antibody is nivolumab. The antibody nivolumab is also known as OPDIVO®.

In some embodiments, the anti-PD-1 antibodies of the present disclosure comprise the CDRs of the antibody pembrolizumab. See U.S. Pat. Nos. 8,354,509 and 8,900,587. In some embodiments, the CDRs of the antibody pembrolizumab are delineated using the Kabat numbering scheme (Kabat, E. A., et al. (1991) Sequences of Proteins of Immunological Interest, Fifth Edition, U.S. Department of Health and Human Services, NTH Publication No. 91-3242). The present disclosure encompasses an anti-PD-1 antibody or derivative thereof comprising a heavy or light chain variable domain, said variable domain comprising (a) a set of three CDRs, in which said set of CDRs are from the monoclonal antibody pembrolizumab, and (b) a set of four framework regions, in which said set of framework regions differs from the set of framework regions in the monoclonal antibody pembrolizumab, and in which said anti-PD-1 antibody or derivative thereof binds to PD-1. In certain embodiments, the anti-PD-1 antibody is pembrolizumab. The antibody pembrolizumab is also known as KEYTRUDA®. (Merck & Co., Inc., Kenilworth, NJ, USA).

In some embodiments, the anti-PD-1 antibody is a monoclonal antibody.

In some embodiments, the anti-PD-1 antibody is nivolumab, which is also known as the antibody OPDIVO® as described in WO 2006/121168.

In some embodiments, the anti-PD-1 antibody is pembrolizumab, which is also known as the antibody KEYTRUDA® as described in U.S. Pat. Nos. 8,354,509 and 8,900,587.

Anti-PD-1 antibodies of the present invention may also be described or specified in terms of their binding affinity to PD-1 (e.g., human PD-1). Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

The antibodies also include derivatives that are modified, i.e., by the covalent attachment of any type of molecule to the antibody such that covalent attachment does not prevent the antibody from binding to PD-1. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Anti-PD-1 antibodies, or antigen-binding fragments thereof, as described herein can be administered by any suitable route and mode. Suitable routes of administering antibodies, or antigen-binding fragments thereof, of the present invention are well known in the art and may be selected by those of ordinary skill in the art. In one embodiment, anti-PD-1 antibodies, or antigen-binding fragments thereof, as described herein are administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of the anti-PD-1 antibody, or antigen-binding fragment thereof, as described herein is intravenous infusion. In some embodiments, the route of administration of the anti-PD-1 antibody, or antigen-binding fragment thereof, as described herein is intravenous injection or infusion. In some embodiments, the route of administration of the anti-PD-1 antibody, or antigen-binding fragment thereof, as described herein is oral.

D. Anti-PD-L1 Antibody

Generally, anti-PD-L1 antibodies or antigen-binding fragments thereof of the disclosure bind to PD-L1, e.g., human PD-L1. In some embodiments, the anti-PD-L1 antibody or antigen-binding fragment thereof comprises the complementary determining regions (CDRs) of an antibody or antigen-binding fragment selected from the group consisting of atezolizumab, BMS-936559, durvalumab, avelumab, envafolimab, CK-301, CS-1001, SHR-1316, CBT-502, and BGB-A333, or a biosimilar thereof. In some embodiments, the anti-PD-L1 antibody or antigen-binding fragment thereof comprises the CDRs of an antibody or antigen-binding fragment selected from the group consisting of atezolizumab, BMS-936559, durvalumab, avelumab, envafolimab, CK-301, CS-1001, SHR-1316, CBT-502, and BGB-A333. In some embodiments, the anti-PD-L1 antibody or antigen-binding fragment thereof comprises the heavy chain variable region and the light chain variable region of an antibody or antigen-binding fragment selected from the group consisting of atezolizumab, BMS-936559, durvalumab, avelumab, envafolimab, CK-301, CS-1001, SHR-1316, CBT-502, and BGB-A333, or a biosimilar thereof. In some embodiments, the anti-PD-L1 antibody or antigen-binding fragment thereof comprises the heavy chain variable region and the light chain variable region of an antibody or antigen-binding fragment selected from the group consisting of atezolizumab, BMS-936559, durvalumab, avelumab, envafolimab, CK-301, CS-1001, SHR-1316, CBT-502, and BGB-A333. In some embodiments, the anti-PD-L1 antibody or antigen-binding fragment thereof is selected from the group consisting of atezolizumab, BMS-936559, durvalumab, avelumab, envafolimab, CK-301, CS-1001, SHR-1316, CBT-502, and BGB-A333, or a biosimilar thereof. In some embodiments, the anti-PD-L1 antibody or antigen-binding fragment thereof is selected from the group consisting of atezolizumab, BMS-936559, durvalumab, avelumab, envafolimab, CK-301, CS-1001, SHR-1316, CBT-502, and BGB-A333. In some embodiments, the anti-PD-L1 antibody or antigen-binding fragment thereof is atezolizumab. See U.S. Pat. No. 8,217,149. The antibody atezolizumab is also known as TECENTRIQ®. In some embodiments, the anti-PD-L1 antibody or antigen-binding fragment thereof is BMS-936559. See U.S. Pat. No. 7,943,743 (referred to as 12A4). In some embodiments, the anti-PD-L1 antibody or antigen-binding fragment thereof is durvalumab. The antibody durvalumab is also known as IMFINZI®. See Akinleye and Rasool, J. Hematol. Oncol., 2019, 12:92. In some embodiments, the anti-PD-L1 antibody or antigen-binding fragment thereof is avelumab. The antibody avelumab is also known as BAVENCIO®. See Akinleye and Rasool, J. Hematol. Oncol., 2019, 12:92. In some embodiments, the anti-PD-L1 antibody or antigen-binding fragment thereof is envafolimab. See Akinleye and Rasool, J. Hematol. Oncol., 2019, 12:92. In some embodiments, the anti-PD-L1 antibody or antigen-binding fragment thereof is CK-301. See Akinleye and Rasool, J. Hematol. Oncol., 2019, 12:92. In some embodiments, the anti-PD-L1 antibody or antigen-binding fragment thereof is CS-1001. See Akinleye and Rasool, J. Hematol. Oncol., 2019, 12:92. In some embodiments, the anti-PD-L1 antibody or antigen-binding fragment thereof is SHR-1316. See Akinleye and Rasool, J. Hematol. Oncol., 2019, 12:92. In some embodiments, the anti-PD-L1 antibody or antigen-binding fragment thereof is CBT-502. See Akinleye and Rasool, J. Hematol. Oncol., 2019, 12:92. In some embodiments, the anti-PD-L1 antibody or antigen-binding fragment thereof is BGB-A333. See Akinleye and Rasool, J. Hematol. Oncol., 2019, 12:92.

Anti-PD-L1 antibodies of the disclosure are preferably monoclonal, and may be multispecific, human, humanized or chimeric antibodies, single chain antibodies, Fab fragments, F(ab′) fragments, fragments produced by a Fab expression library, and PD-L1 binding fragments of any of the above. In some embodiments, an anti-PD-L1 antibody described herein binds specifically to PD-L1 (e.g., human PD-L1). The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the antibodies are antigen-binding fragments (e.g., human antigen-binding fragments) as described herein and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-PD-L1 antibodies or antigen-binding fragments thereof are human, murine (e.g., mouse and rat), donkey, sheep, rabbit, goat, guinea pig, camelid, horse, or chicken.

The anti-PD-L1 antibodies of the present disclosure may be monospecific, bispecific, trispecific or of greater multi specificity. Multispecific antibodies may be specific for different epitopes of PD-L1 or may be specific for both PD-L1 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Anti-PD-L1 antibodies of the present disclosure may be described or specified in terms of the particular CDRs they comprise. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January;27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8;309(3):657-70, (“Aho” numbering scheme); and Martin et al., “Modeling antibody hypervariable loops: a combined algorithm,” PNAS, 1989, 86(23):9268-9272, (“AbM” numbering scheme). The boundaries of a given CDR may vary depending on the scheme used for identification. In some embodiments, a “CDR” or “complementarity determining region,” or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

In some embodiments, numbering of amino acid residues in CDR sequences of anti-PD-L1 antibodies or antigen-binding fragments thereof provided herein are according to the IMGT numbering scheme as described in Lefranc, M. P. et al., Dev. Comp. Immunol., 2003, 27, 55-77.

In some embodiments, numbering of amino acid residues in CDR sequences of anti-PD-L1 antibodies or antigen-binding fragments thereof provided herein are according to the Kabat numbering scheme as described in Kabat, E. A., et al. (1991) Sequences of Proteins of Immunological Interest, Fifth Edition, U.S. Department of Health and Human Services, NTH Publication No. 91-3242.

In some embodiments, the anti-PD-L1 antibodies of the present disclosure comprise the CDRs of the antibody atezolizumab. See U.S. Pat. No. 8,217,149. In some embodiments, the CDRs of the antibody atezolizumab are delineated using the Kabat numbering scheme (Kabat, E. A., et al. (1991) Sequences of Proteins of Immunological Interest, Fifth Edition, U.S. Department of Health and Human Services, NTH Publication No. 91-3242). The present disclosure encompasses an anti-PD-L1 antibody or derivative thereof comprising a heavy or light chain variable domain, said variable domain comprising (a) a set of three CDRs, in which said set of CDRs are from the monoclonal antibody atezolizumab, and (b) a set of four framework regions, in which said set of framework regions differs from the set of framework regions in the monoclonal antibody atezolizumab, and in which said anti-PD-L1 antibody or derivative thereof binds to PD-L1. In certain embodiments, the anti-PD-L1 antibody is atezolizumab. The antibody atezolizumab is also known as TECENTRIQ®.

In some embodiments, the anti-PD-L1 antibodies of the present disclosure comprise the CDRs of the antibody BMS-936559. See U.S. Pat. No. 7,943,743 (referred to as 12A4). In some embodiments, the CDRs of the antibody BMS-936559 are delineated using the Kabat numbering scheme (Kabat, E. A, et al, (1991) Sequences of Proteins of Immunological Interest, Fifth Edition, US. Department of Health and Human Services, NTH Publication No. 91-3242). The present disclosure encompasses an anti-PD-L1 antibody or derivative thereof comprising a heavy or light chain variable domain, said variable domain comprising (a) a set of three CDRs, in which said set of CDRs are from the monoclonal antibody BMS-936559, and (b) a set of four framework regions, in which said set of framework regions differs from the set of framework regions in the monoclonal antibody BMS-936559, and in which said anti-PD-L1 antibody or derivative thereof binds to PD-L1. In certain embodiments, the anti-PD-L1 antibody is BMS-936559.

In some embodiments, the anti-PD-L1 antibodies of the present disclosure comprise the CDRs of the antibody durvalumab. In some embodiments, the CDRs of the antibody durvalumab are delineated using the Kabat numbering scheme (Kabat, E. A., et al. (1991) Sequences of Proteins of Immunological Interest, Fifth Edition, U.S. Department of Health and Human Services, NTH Publication No. 91-3242). The present disclosure encompasses an anti-PD-L1 antibody or derivative thereof comprising a heavy or light chain variable domain, said variable domain comprising (a) a set of three CDRs, in which said set of CDRs are from the monoclonal antibody durvalumab, and (b) a set of four framework regions, in which said set of framework regions differs from the set of framework regions in the monoclonal antibody durvalumab, and in which said anti-PD-L1 antibody or derivative thereof binds to PD-L1. In certain embodiments, the anti-PD-L1 antibody is durvalumab. The antibody durvalumab is also known as IMFINZI®.

In some embodiments, the anti-PD-L1 antibodies of the present disclosure comprise the CDRs of the antibody avelumab. In some embodiments, the CDRs of the antibody avelumab are delineated using the Kabat numbering scheme (Kabat, E A., et al (1991) Sequences of Proteins of Immunological Interest. Fifth Edition, U. S. Department of Health and Human Services, NTH Publication No. 91-3242). The present disclosure encompasses an anti-PD-L1 antibody or derivative thereof comprising a heavy or light chain variable domain, said variable domain comprising (a) a set of three CDRs, in which said set of CDRs are from the monoclonal antibody avelumab, and (b) a set of four framework regions, in which said set of framework regions differs from the set of framework regions in the monoclonal antibody avelumab, and in which said anti-PD-L1 antibody or derivative thereof binds to PD-L1. In certain embodiments, the anti-PD-L1 antibody is avelumab. The antibody avelumab is also known as BAVENCIO®.

In some embodiments, the anti-PD-L1 antibody is a monoclonal antibody.

Anti-PD-L1 antibodies of the present invention may also be described or specified in terms of their binding affinity to PD-L1 (e.g., human PD-L1). Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹M, 10⁻⁹ M, 5×10-10M, 10-10 M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

The antibodies also include derivatives that are modified, i.e., by the covalent attachment of any type of molecule to the antibody such that covalent attachment does not prevent the antibody from binding to PD-L1. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Anti-PD-L1 antibodies, or antigen-binding fragments thereof, as described herein can be administered by any suitable route and mode. Suitable routes of administering antibodies, or antigen-binding fragments thereof, of the present invention are well known in the art and may be selected by those of ordinary skill in the art. In one embodiment, anti-PD-L1 antibodies, or antigen-binding fragments thereof, as described herein are administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of the anti-PD-L1 antibody, or antigen-binding fragment thereof, as described herein is intravenous infusion. In some embodiments, the route of administration of the anti-PD-L1 antibody, or antigen-binding fragment thereof, as described herein is intravenous injection or infusion. In some embodiments, the route of administration of the anti-PD-L1 antibody, or antigen-binding fragment thereof, as described herein is oral.

E. Anti-CTLA4 Antibody

Generally, anti-CTLA4 antibodies or antigen-binding fragments thereof of the disclosure bind to cytotoxic T-lymphocyte-associated protein 4 (CTLA4), e.g., human CTLA4. In some embodiments, the anti-CTLA4 antibody or antigen-binding fragment thereof comprises the complementary determining regions (CDRs) of ipilimumab or a biosimilar thereof. In some embodiments, the anti-CTLA4 antibody or antigen-binding fragment thereof comprises the CDRs of ipilimumab. In some embodiments, the anti-CTLA4 antibody or antigen-binding fragment thereof comprises the heavy chain variable region and the light chain variable region of ipilimumab or a biosimilar thereof. In some embodiments, the anti-CTLA4 antibody or antigen-binding fragment thereof comprises the heavy chain variable region and the light chain variable region of ipilimumab. In some embodiments, the anti-CTLA4 antibody or antigen-binding fragment ipilimumab or a biosimilar thereof. In some embodiments, the anti-CTLA4 antibody or antigen-binding fragment thereof is ipilimumab. The antibody ipilimumab is also known as YERVOY®.

Anti-CTLA4 antibodies of the disclosure are preferably monoclonal, and may be multispecific, human, humanized or chimeric antibodies, single chain antibodies, Fab fragments, F(ab′) fragments, fragments produced by a Fab expression library, and CTLA4 binding fragments of any of the above. In some embodiments, an anti-CTLA4 antibody described herein binds specifically to CTLA4 (e.g., human CTLA4). The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the antibodies are antigen-binding fragments (e.g., human antigen-binding fragments) as described herein and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-CTLA4 antibodies or antigen-binding fragments thereof are human, murine (e.g., mouse and rat), donkey, sheep, rabbit, goat, guinea pig, camelid, horse, or chicken.

The anti-CTLA4 antibodies of the present disclosure may be monospecific, bispecific, trispecific or of greater multi specificity. Multispecific antibodies may be specific for different epitopes of CTLA4 or may be specific for both CTLA4 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Anti-CTLA4 antibodies of the present disclosure may be described or specified in terms of the particular CDRs they comprise. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January;27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8;309(3):657-70, (“Aho” numbering scheme); and Martin et al., “Modeling antibody hypervariable loops: a combined algorithm,” PNAS, 1989, 86(23):9268-9272, (“AbM” numbering scheme). The boundaries of a given CDR may vary depending on the scheme used for identification. In some embodiments, a “CDR” or “complementarity determining region,” or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

In some embodiments, numbering of amino acid residues in CDR sequences of anti-CTLA4 antibodies or antigen-binding fragments thereof provided herein are according to the IMGT numbering scheme as described in Lefranc, M. P. et al., Dev. Comp. Immunol., 2003, 27, 55-77.

In some embodiments, the anti-CTLA4 antibodies of the present disclosure comprise the CDRs of the antibody ipilimumab. In some embodiments, the CDRs of the antibody ipilimumab are delineated using the Kabat numbering scheme (Kabat, E. A., et al (1991) Sequences of Proteins of immunological Interest, Fifth Edition, U S Department of Health and Human Services, NTH Publication No. 91-3242). The present disclosure encompasses an anti-CTLA4 antibody or derivative thereof comprising a heavy or light chain variable domain, said variable domain comprising (a) a set of three CDRs, in which said set of CDRs are from the monoclonal antibody ipilimumab, and (b) a set of four framework regions, in which said set of framework regions differs from the set of framework regions in the monoclonal antibody ipilimumab, and in which said anti-CTLA4 antibody or derivative thereof binds to CTLA4. In certain embodiments, the anti-CTLA4 antibody is ipilimumab. The antibody ipilimumab is also known as YERVOY®.

In some embodiments, the anti-CTLA4 antibody is a monoclonal antibody.

Anti-CTLA4 antibodies of the present invention may also be described or specified in terms of their binding affinity to CTLA4 (e.g., human CTLA4). Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹M, 10⁻⁹ M, 5×10-10M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10-14 M, 10-14 M, 5×10⁻¹⁵ M, or 10⁻¹⁵ M.

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

The antibodies also include derivatives that are modified, i.e., by the covalent attachment of any type of molecule to the antibody such that covalent attachment does not prevent the antibody from binding to CTLA4. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Anti-CTLA4 antibodies, or antigen-binding fragments thereof, as described herein can be administered by any suitable route and mode. Suitable routes of administering antibodies, or antigen-binding fragments thereof, of the present invention are well known in the art and may be selected by those of ordinary skill in the art. In one embodiment, anti-CTLA4 antibodies, or antigen-binding fragments thereof, as described herein are administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of the anti-CTLA4 antibody, or antigen-binding fragment thereof, as described herein is intravenous infusion. In some embodiments, the route of administration of the anti-CTLA4 antibody, or antigen-binding fragment thereof, as described herein is intravenous injection or infusion. In some embodiments, the route of administration of the anti-CTLA4 antibody, or antigen-binding fragment thereof, as described herein is oral.

F. Nucleic Acids, Host Cells and Methods of Production

In some aspects, also provided herein are nucleic acids encoding an anti-PD-1 antibody or antigen-binding fragment thereof as described herein, an anti-PD-L1 antibody or antigen-binding fragment thereof as described herein, or an anti-CTLA4 antibody or antigen-binding fragment thereof as described herein. Further provided herein are vectors comprising the nucleic acids encoding an anti-PD-1 antibody or antigen-binding fragment thereof as described herein, an anti-PD-L1 antibody or antigen-binding fragment thereof as described herein, or an anti-CTLA4 antibody or antigen-binding fragment thereof as described herein. Further provided herein are host cells expressing the nucleic acids encoding an anti-PD-1 antibody or antigen-binding fragment thereof as described herein, an anti-PD-L1 antibody or antigen-binding fragment thereof as described herein, or an anti-CTLA4 antibody or antigen-binding fragment thereof as described herein. Further provided herein are host cells comprising the vectors comprising the nucleic acids encoding an anti-PD-1 antibody or antigen-binding fragment thereof as described herein, an anti-PD-L1 antibody or antigen-binding fragment thereof as described herein or an anti-CTLA4 antibody or antigen-binding fragment thereof as described herein.

The anti-PD-1 antibodies described herein, anti-PD-L1 antibodies described herein, or anti-CTLA4 antibodies described herein may be prepared by well-known recombinant techniques using well known expression vector systems and host cells. In one embodiment, the antibodies are prepared in a CHO cell using the GS expression vector system as disclosed in De la Cruz Edmunds et al., 2006, Molecular Biotechnology 34; 179-190, EP216846, U.S. Pat. No. 5,981,216, WO 87/04462, EP323997, U.S. Pat. Nos. 5,591,639, 5,658,759, EP338841, U.S. Pat. Nos. 5,879,936, and 5,891,693.

Monoclonal anti-PD-1 antibodies described herein, anti-PD-L1 antibodies described herein, or anti-CTLA4 antibodies described herein may e.g. be produced by the hybridoma method first described by Kohler et al., Nature, 256, 495 (1975), or may be produced by recombinant DNA methods. Monoclonal antibodies may also be isolated from phage antibody libraries using the techniques described in, for example, Clackson et al., Nature, 352, 624-628 (1991) and Marks et al., J. Mol. Biol., 222(3):581-597 (1991). Monoclonal antibodies may be obtained from any suitable source. Thus, for example, monoclonal antibodies may be obtained from hybridomas prepared from murine splenic B cells obtained from mice immunized with an antigen of interest, for instance in form of cells expressing the antigen on the surface, or a nucleic acid encoding an antigen of interest. Monoclonal antibodies may also be obtained from hybridomas derived from antibody-expressing cells of immunized humans or non-human mammals such as rats, dogs, primates, etc.

In one embodiment, the antibody (e.g., anti-PD-1 antibody, anti-PD-L1 antibody, or anti-CTLA4 antibody) of the invention is a human antibody. Human monoclonal antibodies directed against PD-1, PD-L1, or CTLA4 may be generated using transgenic or transchromosomal mice carrying parts of the human immune system rather than the mouse system. Such transgenic and transchromosomic mice include mice referred to herein as HuMAb mice and KM mice, respectively, and are collectively referred to herein as “transgenic mice”.

The HuMAb mouse contains a human immunoglobulin gene minilocus that encodes unrearranged human heavy (μ and γ) and κ light chain immunoglobulin sequences, together with targeted mutations that inactivate the endogenous μ and κ chain loci (Lonberg, N. et al., Nature, 368, 856-859 (1994)). Accordingly, the mice exhibit reduced expression of mouse IgM or κ and in response to immunization, the introduced human heavy and light chain transgenes undergo class switching and somatic mutation to generate high affinity human IgG,κ monoclonal antibodies (Lonberg, N. et al. (1994), supra; reviewed in Lonberg, N. Handbook of Experimental Pharmacology 113, 49-101 (1994), Lonberg, N. and Huszar. D., Intern. Rev. Immunol, Vol. 13 65-93 (1995) and Harding, F. and Lonberg, N. Ann, N.Y. Acad. Sci 764:536-546 (1995)). The preparation of HuMAb mice is described in detail in Taylor, L. et al., Nucleic Acids Research. 20:6287-6295 (1992), Chen, J. et al., International Immunology. 5:647-656 (1993), Tuaillon at al., J. Immunol, 152:2912-2920 (1994), Taylor, L. et al., International Immunology, 6:579-591 (1994), Fishwild, D. et al., Nature Biotechnology, 14:845-851 (1996). See also U.S. Pat. Nos. 5,545,806, 5,569,825, 5,625,126, 5,633,425, 5,789,650, 5,877,397, 5,661,016, 5,814,318, 5,874,299, 5,770,429, 5,545,807, WO 98/24884, WO 94/25585, WO 93/1227, WO 92/22645, WO 92/03918 and WO 01/09187.

The HCo7 mice have a JKD disruption in their endogenous light chain (kappa) genes (as described in Chen et al, EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCo7 human heavy chain transgene (as described in U.S. Pat. No. 5,770,429).

The HCo12 mice have a JKD disruption in their endogenous light chain (kappa) genes (as described in Chen et al., EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCo12 human heavy chain transgene (as described in Example 2 of WO 01/14424).

The HCo17 transgenic mouse strain (see also US 2010/0077497) was generated by coinjection of the 80 kb insert of pHC2 (Taylor et al. (1994) Int. Immunol., 6:579-591), the Kb insert of pVX6, and a −460 kb yeast artificial chromosome fragment of the yIgH24 chromosome. This line was designated (HCo17) 25950. The (HCo17) 25950 line was then bred with mice comprising the CMD mutation (described in Example 1 of PCT Publication WO 01109187), the JKD mutation (Chen et al, (1993) EMBO J. 12:811-820), and the (KC05) 9272 transgene (Fishwild et al. (1996) Nature Biotechnology, 14:845-851). The resulting mice express human immunoglobulin heavy and kappa light chain trans genes in a background homozygous for disruption of the endogenous mouse heavy and kappa light chain loci.

The HCo20 transgenic mouse strain is the result of a co-injection of minilocus 30 heavy chain transgene pHC2, the germline variable region (Vh)-containing YAC yIgH10, and the minilocus construct pVx6 (described in WO09097006). The (HCo20) line was then bred with mice comprising the CMD mutation (described in Example 1 of PCT Publication WO 01/09187), the JKD mutation (Chen et al. (1993) EMBO J. 12:811-820), and the (KC05) 9272 trans gene (Fishwild eta). (1996) Nature Biotechnology, 14:845-851). The resulting mice express human 10 immunoglobulin heavy and kappa light chain transgenes in a background homozygous for disruption of the endogenous mouse heavy and kappa light chain loci.

In order to generate HuMab mice with the salutary effects of the Ba1b/c strain, HuMab mice were crossed with KCO05 [MIK] (Ba1b) mice which were generated by backcrossing the KC05 strain (as described in Fishwild et (1996) Nature Biotechnology, 14:845-851) to wild-type Ba1b/c mice to generate mice as described in WO09097006. Using this crossing Ba1b/c hybrids were created for HCo12, HCo17, and HCo20 strains.

In the KM mouse strain, the endogenous mouse kappa light chain gene has been homozygously disrupted as described in Chen et al., EMBO J. 12:811-820 (1993) and the endogenous mouse heavy chain gene has been homozygously disrupted as described in Example 1 of WO 01/09187, This mouse strain carries a human kappa light chain transgene, KCo5, as described in Fishwild et al., Nature Biotechnology, 14:845-851 (1996). This mouse strain also carries a human heavy chain transchromosome composed of chromosome 14 fragment hCF (SC20) as described in WO 02/43478.

Splenocytes from these transgenic mice may be used to generate hybridomas that secrete human monoclonal antibodies according to well-known techniques. Human monoclonal or polyclonal antibodies of the invention, or antibodies of the invention originating from other species may also be generated transgenically through the generation of another non-human mammal or plant that is transgenic for the immunoglobulin heavy and light chain sequences of interest and production of the antibody in a recoverable form therefrom. In connection with the transgenic production in mammals, antibodies may be produced in, and recovered from, the milk of goats, cows, or other mammals. See for instance U.S. Pat. Nos. 5,827,690, 5,756,687, 5,750,172 and 5,741,957.

Further, human antibodies of the invention or antibodies of the invention from other species may be generated through display-type technologies, including, without limitation, phage display, retroviral display, ribosomal display, and other techniques, using techniques well known in the art and the resulting molecules may be subjected to additional maturation, such as affinity maturation, as such techniques are well known in the art (See for instance Hoogenboom et al., J. Mol, Biol. 227(2):381-388 (1992) (phage display), Vaughan et al., Nature Biotech, 14:309 (1996) (phage display), Hanes and Plucthau, PNAS USA 94:4937-4942 (1997) (ribosomal display), Parmley and Smith, Gene, 73:305-318 (1988) (phage display), Scott, TIBS. 17:241-245 (1992), Cwirla et al., PNAS USA, 87:6378-6382 (1990), Russel et al., Nucl. Acids Research, 21:1081-4085 (1993), Hogenboom et al., Immunol, Reviews, 130:43-68 (1992), Chiswell and McCafferty, TIBTECH, 10:80-84 (1992), and U.S. Pat. No. 5,733,743). If display technologies are utilized to produce antibodies that are not human, such antibodies may be humanized.

G. Treatment Outcome

In some embodiments, treating the subject comprises inhibiting cancer cell growth, inhibiting cancer cell proliferation, inhibiting cancer cell migration, inhibiting cancer cell invasion, decreasing or eliminating one or more signs or symptoms of cancer, reducing the size (e.g., volume) of a cancer tumor, reducing the number of cancer tumors, reducing the number of cancer cells, inducing cancer cell necrosis, pyroptosis, oncosis, apoptosis, autophagy, or other cell death, increasing survival time of the subject, or enhancing the therapeutic effects of another drug or therapy.

In some embodiments, treating the subject as described herein results in a tumor growth inhibition (TGI) index that is between about 10% and 70% (e.g., about 10%, 15%, 20%, 25%, 30%, 35%, 40%, 45%, 50%, 55%, 60%, 65%, or 70%). Preferably, treating the subject results in a TGI index that is at least about 70% (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%). More preferably, treating the subject results in a TGI index that is at least about 85% (e.g., about 85%, 86%, 87%, 88%, 89%, 90%, 91%, 92%, 93%, 94%, 95%, 96%, 97%, 98%, 99%, or 100%). Even more preferably, treating the subject results in a TGI index that is at least about 95% (e.g., about 95%, 96%, 97%, 98%, 99%, or 100%). Most preferably, treating the subject results in a TGI index that is about 100% or more (e.g., about 100%, 101%, 102%, 103%, 104%, 105%, 106%, 107%, 108%, 109%, 110%, 111%, 112%, 113%, 114%, 115%, 116%, 117%, 118%, 119%, 120%, 125%, 130%, 135%, 140%, 145%, 150%, or more).

In particular embodiments, treating the subject with tucatinib, or salt or solvate thereof, and an anti-PD-1 antibody or antigen-binding fragment thereof results in a TGI index that is greater than the TGI index that is observed when tucatinib, or salt or solvate thereof, or an anti-PD-1 antibody or antigen-binding fragment thereof is used alone. In some instances, treating the subject results in a TGI index that is greater than the TGI index that is observed when tucatinib, or salt or solvate thereof, is used alone. In other instances, treating the subject results in a TGI index that is greater than the TGI index that is observed when an anti-PD-1 antibody or antigen-binding fragment thereof is used alone. In some embodiments, treating the subject results in a TGI index that is at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% greater than the TGI index that is observed when tucatinib, or salt or solvate thereof, or an anti-PD-1 antibody or antigen-binding fragment thereof is used alone.

In particular embodiments, treating the subject with tucatinib, or salt or solvate thereof, and an anti-PD-L1 antibody or antigen-binding fragment thereof results in a TGI index that is greater than the TGI index that is observed when tucatinib, or salt or solvate thereof, or an anti-PD-L1 antibody or antigen-binding fragment thereof is used alone. In some instances, treating the subject results in a TGI index that is greater than the TGI index that is observed when tucatinib, or salt or solvate thereof, is used alone. In other instances, treating the subject results in a TGI index that is greater than the TGI index that is observed when an anti-PD-L1 antibody or antigen-binding fragment thereof is used alone. In some embodiments, treating the subject results in a TGI index that is at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% greater than the TGI index that is observed when tucatinib, or salt or solvate thereof, or an anti-PD-L1 antibody or antigen-binding fragment thereof is used alone.

In some embodiments, the combination of the tucatinib, or salt or solvate thereof, and an anti-PD-1 antibody or antigen-binding fragment thereof is synergistic. In particular embodiments, with respect to the synergistic combination, treating the subject results in a TGI index that is greater than the TGI index that would be expected if the combination of tucatinib or salt or solvate thereof, and an anti-PD-1 antibody or antigen-binding fragment thereof produced an additive effect. In some instances, the TGI index observed when a combination of tucatinib, or salt or solvate thereof, an anti-PD-1 antibody or antigen-binding fragment thereof is administered is at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% greater than the TGI index that would be expected if the combination of tucatinib, or salt or solvate thereof, and an anti-PD-1 antibody or antigen-binding fragment thereof produced an additive effect.

In some embodiments, the combination of the tucatinib, or salt or solvate thereof, and an anti-PD-L1 antibody or antigen-binding fragment thereof is synergistic. In particular embodiments, with respect to the synergistic combination, treating the subject results in a TGI index that is greater than the TGI index that would be expected if the combination of tucatinib or salt or solvate thereof, and an anti-PD-L1 antibody or antigen-binding fragment thereof produced an additive effect. In some instances, the TGI index observed when a combination of tucatinib, or salt or solvate thereof, an anti-PD-L1 antibody or antigen-binding fragment thereof is administered is at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% greater than the TGI index that would be expected if the combination of tucatinib, or salt or solvate thereof, and an anti-PD-L1 antibody or antigen-binding fragment thereof produced an additive effect.

In one aspect, a method of treating cancer with tucatinib as described herein and an anti-PD-1 antibody as described herein results in an improvement in one or more therapeutic effects in the subject after administration of tucatinib as described herein and the anti-PD-1 antibody as described herein relative to a baseline. In some embodiments, the one or more therapeutic effects is the size of the tumor derived from the solid tumor, the objective response rate, the duration of response, the time to response, progression free survival, overall survival, or any combination thereof. In one embodiment, the one or more therapeutic effects is the size of the tumor derived from the solid tumor. In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression.

In one embodiment of the methods or uses or product for uses provided herein, response to treatment with tucatinib as described herein and an anti-PD-1 antibody as described herein may include the following criteria (RECIST Criteria 1.1):

| | Category | Criteria |
|---|---|---|
| Based on | Complete | Disappearance of all target lesions. Any pathological |
| target lesions | Response (CR) | lymph nodes must have reduction in short axis to <10 mm. |
| | Partial | ≥30% decrease in the sum of the longest diameter |
| | Response (PR) | (LD) of target lesions, taking as reference the baseline |
| | | sum of LDs. |
| | Stable | Neither sufficient shrinkage to qualify for PR nor |
| | Disease (SD) | sufficient increase to qualify for PD, taking as |
| | | reference the smallest sum of LDs while in trial. |
| | Progressive | ≥20% (and ≥5 mm) increase in the sum of the LDs of |
| | Disease (PD) | target lesions, taking as reference the smallest sum of |
| | | the target LDs recorded while in trial or the appearance |
| | | of one or more new lesions. |
| Based on non- | CR | Disappearance of all non-target lesions and |
| target lesions | | normalization of tumor marker level. All lymph nodes |
| | | must be non-pathological in size (<10 mm short axis). |
| | SD | Persistence of one or more non-target lesion(s) or/and |
| | | maintenance of tumor marker level above the normal |
| | | limits. |
| | PD | Appearance of one or more new lesions and/or |
| | | unequivocal progression of existing non-target lesions. |

In one embodiment of the methods or uses or product for uses provided herein, the effectiveness of treatment with tucatinib described herein and an anti-PD-1 antibody described herein is assessed by measuring the objective response rate. In some embodiments, the objective response rate is the proportion of patients with tumor size reduction of a predefined amount and for a minimum period of time. In some embodiments, the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses provided herein, response to treatment with tucatinib described herein and an anti-PD-1 antibody described herein is assessed by measuring the size of a tumor derived from the cancer described herein (e.g., solid tumor). In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of tucatinib described herein and/or anti-PD-1 antibody described herein. In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of tucatinib described herein and/or anti-PD-1 antibody described herein. In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%. In one embodiment, the size of a tumor derived from the cancer is reduced by 100%. In one embodiment, the size of a tumor derived from the cancer is measured by magnetic resonance imaging (MRI). In one embodiment, the size of a tumor derived from the cancer is measured by computed tomography (CT). In one embodiment, the size of a tumor derived from the cancer is measured by positron emission tomography (PET). In one embodiment, the size of a tumor derived from the cancer is measured by mammography. In one embodiment, the size of a tumor derived from the cancer is measured by sonography. See Gruber et. al., 2013, BMC Cancer. 13:328.

In one embodiment of the methods or uses or product for uses provided described herein, response to treatment with tucatinib described herein and an anti-PD-1 antibody described herein, promotes regression of a tumor derived from the cancer described herein (e.g., solid tumor). In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the tucatinib described herein and/or anti-PD-1 antibody described herein. In one embodiment, a tumor derived from the cancer regresses by at least about 10% to about 80%. In one embodiment, a tumor derived from the cancer regresses by at least about 20% to about 80%. In one embodiment, a tumor derived from the cancer regresses by at least about 30% to about 80%. In one embodiment, a tumor derived from the cancer regresses by at least about 40% to about 80%. In one embodiment, a tumor derived from the cancer regresses by at least about 50% to about 80%. In one embodiment, a tumor derived from the cancer regresses by at least about 60% to about 80%. In one embodiment, a tumor derived from the cancer regresses by at least about 70% to about 80%. In one embodiment, a tumor derived from the cancer regresses by at least about 80%. In one embodiment, a tumor derived from the cancer regresses by at least about 85%. In one embodiment, a tumor derived from the cancer regresses by at least about 90%. In one embodiment, a tumor derived from the cancer regresses by at least about 95%. In one embodiment, a tumor derived from the cancer regresses by at least about 98%. In one embodiment, a tumor derived from the cancer regresses by at least about 99%. In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of tucatinib described herein and/or an anti-PD-1 antibody described herein. In one embodiment, a tumor derived from the cancer regresses by at least 10% to 80%. In one embodiment, a tumor derived from the cancer regresses by at least 20% to 80%. In one embodiment, a tumor derived from the cancer regresses by at least 30% to 80%. In one embodiment, a tumor derived from the cancer regresses by at least 40% to 80%. In one embodiment, a tumor derived from the cancer regresses by at least 50% to 80%. In one embodiment, a tumor derived from the cancer regresses by at least 60% to 80%. In one embodiment, a tumor derived from the cancer regresses by at least 70% to 80%. In one embodiment, a tumor derived from the cancer regresses by at least 80%. In one embodiment, a tumor derived from the cancer regresses by at least 85%. In one embodiment, a tumor derived from the cancer regresses by at least 90%. In one embodiment, a tumor derived from the cancer regresses by at least 95%. In one embodiment, a tumor derived from the cancer regresses by at least 98%. In one embodiment, a tumor derived from the cancer regresses by at least 99%. In one embodiment, a tumor derived from the cancer regresses by 100%. In one embodiment, regression of a tumor is determined by magnetic resonance imaging (MRI). In one embodiment, regression of a tumor is determined by computed tomography (CT). In one embodiment, regression of a tumor is determined by positron emission tomography (PET). In one embodiment, regression of a tumor is determined by mammography. In one embodiment, regression of a tumor is determined by sonography. See Gruber et. al., 2013, BMC Cancer. 13:328.

In one embodiment of the methods or uses or product for uses described herein, response to treatment with tucatinib described and an anti-PD-1 antibody described herein herein is assessed by measuring the time of progression free survival after administration of tucatinib described herein and/or anti-PD-1 antibody described herein. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib described herein and/or anti-PD-1 antibody described herein. In some embodiments, the subject exhibits progression-free survival of at least about 6 months after administration of tucatinib described herein and/or anti-PD-1 antibody described herein. In some embodiments, the subject exhibits progression-free survival of at least about one year after administration of tucatinib described herein and/or anti-PD-1 antibody described herein. In some embodiments, the subject exhibits progression-free survival of at least about two years after administration of tucatinib described herein and/or anti-PD-1 antibody described herein. In some embodiments, the subject exhibits progression-free survival of at least about three years after administration of tucatinib described herein and/or anti-PD-1 antibody described herein. In some embodiments, the subject exhibits progression-free survival of at least about four years after administration of tucatinib described herein and/or anti-PD-1 antibody described herein. In some embodiments, the subject exhibits progression-free survival of at least about five years after administration of tucatinib described herein and/or anti-PD-1 antibody described herein. In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of tucatinib described herein and/or anti-PD-1 antibody described herein. In some embodiments, the subject exhibits progression-free survival of at least 6 months after administration of tucatinib described herein and/or anti-PD-1 antibody described herein. In some embodiments, the subject exhibits progression-free survival of at least one year after administration of tucatinib described herein and/or anti-PD-1 antibody described herein. In some embodiments, the subject exhibits progression-free survival of at least two years after administration of tucatinib described herein and/or anti-PD-1 antibody described herein. In some embodiments, the subject exhibits progression-free survival of at least three years after administration of tucatinib described herein and/or anti-PD-1 antibody described herein. In some embodiments, the subject exhibits progression-free survival of at least four years after administration of tucatinib described herein and/or anti-PD-1 antibody described herein. In some embodiments, the subject exhibits progression-free survival of at least five years after administration of tucatinib described herein and/or anti-PD-1 antibody described herein.

In one embodiment of the methods or uses or product for uses described herein, response to treatment with tucatinib described herein and an anti-PD-1 antibody described herein is assessed by measuring the time of overall survival after administration of tucatinib described herein and/or anti-PD-1 antibody described herein. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib described herein and/or anti-PD-1 antibody described herein. In some embodiments, the subject exhibits overall survival of at least about 6 months after administration of tucatinib described herein and/or anti-PD-1 antibody described herein. In some embodiments, the subject exhibits overall survival of at least about one year after administration of tucatinib described herein and/or anti-PD-1 antibody described herein. In some embodiments, the subject exhibits overall survival of at least about two years after administration of tucatinib described herein and/or anti-PD-1 antibody described herein. In some embodiments, the subject exhibits overall survival of at least about three years after administration of tucatinib described herein and/or anti-PD-1 antibody described herein. In some embodiments, the subject exhibits overall survival of at least about four years after administration of tucatinib described herein and/or anti-PD-1 antibody described herein. In some embodiments, the subject exhibits overall survival of at least about five years after administration of tucatinib described herein and/or anti-PD-1 antibody described herein. In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least about 12 months, at least eighteen months, at least two years, at least three years, at least four years, or at least five years after administration of tucatinib described herein and/or anti-PD-1 antibody described herein. In some embodiments, the subject exhibits overall survival of at least 6 months after administration of tucatinib described herein and/or anti-PD-1 antibody described herein. In some embodiments, the subject exhibits overall survival of at least one year after administration of tucatinib described herein and/or anti-PD-1 antibody described herein. In some embodiments, the subject exhibits overall survival of at least two years after administration of tucatinib described herein and/or anti-PD-1 antibody described herein. In some embodiments, the subject exhibits overall survival of at least three years after administration of tucatinib described herein and/or anti-PD-1 antibody described herein. In some embodiments, the subject exhibits overall survival of at least four years after administration of tucatinib described herein and/or anti-PD-1 antibody described herein. In some embodiments, the subject exhibits overall survival of at least five years after administration of tucatinib described herein and/or anti-PD-1 antibody described herein.

In one embodiment of the methods or uses or product for uses described herein, response to treatment with tucatinib described herein and an anti-PD-1 antibody described herein is assessed by measuring the duration of response to tucatinib described herein and anti-PD-1 antibody described herein after administration of tucatinib described herein and/or anti-PD-1 antibody described herein. In some embodiments, the duration of response to tucatinib described herein and an anti-PD-1 antibody described herein is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib described herein and/or anti-PD-1 antibody described herein. In some embodiments, the duration of response to tucatinib described herein and an anti-PD-1 antibody described herein is at least about 6 months after administration of tucatinib described herein and/or anti-PD-1 antibody described herein. In some embodiments, the duration of response to tucatinib described herein and an anti-PD-1 antibody described herein is at least about one year after administration of tucatinib described herein and/or anti-PD-1 antibody described herein. In some embodiments, the duration of response to tucatinib described herein and an anti-PD-1 antibody described herein is at least about two years after administration of tucatinib described herein and/or anti-PD-1 antibody described herein. In some embodiments, the duration of response to tucatinib described herein and an anti-PD-1 antibody described herein is at least about three years after administration of tucatinib described herein and/or anti-PD-1 antibody described herein. In some embodiments, the duration of response to tucatinib described herein and an anti-PD-1 antibody described herein is at least about four years after administration of tucatinib described herein and/or anti-PD-1 antibody described herein. In some embodiments, the duration of response to tucatinib described herein and an anti-PD-1 antibody described herein is at least about five years after administration of tucatinib described herein and/or anti-PD-1 antibody described herein. In some embodiments, the duration of response to tucatinib described herein and an anti-PD-1 antibody described herein is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of tucatinib described herein and/or anti-PD-1 antibody described herein. In some embodiments, the duration of response to tucatinib described herein and an anti-PD-1 antibody described herein is at least 6 months after administration of tucatinib described herein and/or anti-PD-1 antibody described herein. In some embodiments, the duration of response to tucatinib described herein and an anti-PD-1 antibody described herein is at least one year after administration of tucatinib described herein and/or anti-PD-1 antibody described herein. In some embodiments, the duration of response to tucatinib described herein and an anti-PD-1 antibody described herein is at least two years after administration of tucatinib described herein and/or anti-PD-1 antibody described herein. In some embodiments, the duration of response to tucatinib described herein and an anti-PD-1 antibody described herein is at least three years after administration of tucatinib described herein and/or anti-PD-1 antibody described herein. In some embodiments, the duration of response to tucatinib described herein and an anti-PD-1 antibody described herein is at least four years after administration of tucatinib described herein and/or anti-PD-1 antibody described herein. In some embodiments, the duration of response to tucatinib described herein and an anti-PD-1 antibody described herein is at least five years after administration of tucatinib described herein and/or anti-PD-1 antibody described herein.

In one aspect, a method of treating cancer with tucatinib as described herein and an anti-PD-L1 antibody as described herein results in an improvement in one or more therapeutic effects in the subject after administration of tucatinib as described herein and the anti-PD-L1 antibody as described herein relative to a baseline. In some embodiments, the one or more therapeutic effects is the size of the tumor derived from the solid tumor, the objective response rate, the duration of response, the time to response, progression free survival, overall survival, or any combination thereof. In one embodiment, the one or more therapeutic effects is the size of the tumor derived from the solid tumor. In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression.

In one embodiment of the methods or uses or product for uses provided herein, response to treatment with tucatinib as described herein and an anti-PD-L1 antibody as described herein may include the following criteria (RECIST Criteria 1.1):

| | Category | Criteria |
|---|---|---|
| Based on | Complete | Disappearance of all target lesions. Any pathological |
| target lesions | Response (CR) | lymph nodes must have reduction in short axis to <10 mm. |
| | Partial | ≥30% decrease in the sum of the longest diameter |
| | Response (PR) | (LD) of target lesions, taking as reference the baseline |
| | | sum of LDs. |
| | Stable | Neither sufficient shrinkage to qualify for PR nor |
| | Disease (SD) | sufficient increase to qualify for PD, taking as |
| | | reference the smallest sum of LDs while in trial. |
| | Progressive | ≥20% (and ≥5 mm) increase in the sum of the LDs of |
| | Disease (PD) | target lesions, taking as reference the smallest sum of |
| | | the target LDs recorded while in trial or the appearance |
| | | of one or more new lesions. |
| Based on non- | CR | Disappearance of all non-target lesions and |
| target lesions | | normalization of tumor marker level. All lymph nodes |
| | | must be non-pathological in size (<10 mm short axis). |
| | SD | Persistence of one or more non-target lesion(s) or/and |
| | | maintenance of tumor marker level above the normal |
| | | limits. |
| | PD | Appearance of one or more new lesions and/or |
| | | unequivocal progression of existing non-target lesions. |

In one embodiment of the methods or uses or product for uses provided herein, the effectiveness of treatment with tucatinib described herein and an anti-PD-L1 antibody described herein is assessed by measuring the objective response rate. In some embodiments, the objective response rate is the proportion of patients with tumor size reduction of a predefined amount and for a minimum period of time. In some embodiments, the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses provided herein, response to treatment with tucatinib described herein and an anti-PD-L1 antibody described herein is assessed by measuring the size of a tumor derived from the cancer described herein (e.g., solid tumor). In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of tucatinib described herein and/or anti-PD-L1 antibody described herein. In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of tucatinib described herein and/or anti-PD-L1 antibody described herein. In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%. In one embodiment, the size of a tumor derived from the cancer is reduced by 100%. In one embodiment, the size of a tumor derived from the cancer is measured by magnetic resonance imaging (MRI). In one embodiment, the size of a tumor derived from the cancer is measured by computed tomography (CT). In one embodiment, the size of a tumor derived from the cancer is measured by positron emission tomography (PET). In one embodiment, the size of a tumor derived from the cancer is measured by mammography. In one embodiment, the size of a tumor derived from the cancer is measured by sonography. See Gruber et. al., 2013, BMC Cancer. 13:328.

In one embodiment of the methods or uses or product for uses provided described herein, response to treatment with tucatinib described herein and an anti-PD-L1 antibody described herein, promotes regression of a tumor derived from the cancer described herein (e.g., solid tumor). In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the tucatinib described herein and/or anti-PD-L1 antibody described herein. In one embodiment, a tumor derived from the cancer regresses by at least about 10% to about 80%. In one embodiment, a tumor derived from the cancer regresses by at least about 20% to about 80%. In one embodiment, a tumor derived from the cancer regresses by at least about 30% to about 80%. In one embodiment, a tumor derived from the cancer regresses by at least about 40% to about 80%. In one embodiment, a tumor derived from the cancer regresses by at least about 50% to about 80%. In one embodiment, a tumor derived from the cancer regresses by at least about 60% to about 80%. In one embodiment, a tumor derived from the cancer regresses by at least about 70% to about 80%. In one embodiment, a tumor derived from the cancer regresses by at least about 80%. In one embodiment, a tumor derived from the cancer regresses by at least about 85%. In one embodiment, a tumor derived from the cancer regresses by at least about 90%. In one embodiment, a tumor derived from the cancer regresses by at least about 95%. In one embodiment, a tumor derived from the cancer regresses by at least about 98%. In one embodiment, a tumor derived from the cancer regresses by at least about 99%. In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of tucatinib described herein and/or an anti-PD-L1 antibody described herein. In one embodiment, a tumor derived from the cancer regresses by at least 10% to 80%. In one embodiment, a tumor derived from the cancer regresses by at least 20% to 80%. In one embodiment, a tumor derived from the cancer regresses by at least 30% to 80%. In one embodiment, a tumor derived from the cancer regresses by at least 40% to 80%. In one embodiment, a tumor derived from the cancer regresses by at least 50% to 80%. In one embodiment, a tumor derived from the cancer regresses by at least 60% to 80%. In one embodiment, a tumor derived from the cancer regresses by at least 70% to 80%. In one embodiment, a tumor derived from the cancer regresses by at least 80%. In one embodiment, a tumor derived from the cancer regresses by at least 85%. In one embodiment, a tumor derived from the cancer regresses by at least 90%. In one embodiment, a tumor derived from the cancer regresses by at least 95%. In one embodiment, a tumor derived from the cancer regresses by at least 98%. In one embodiment, a tumor derived from the cancer regresses by at least 99%. In one embodiment, a tumor derived from the cancer regresses by 100%. In one embodiment, regression of a tumor is determined by magnetic resonance imaging (MRI). In one embodiment, regression of a tumor is determined by computed tomography (CT). In one embodiment, regression of a tumor is determined by positron emission tomography (PET). In one embodiment, regression of a tumor is determined by mammography. In one embodiment, regression of a tumor is determined by sonography. See Gruber et. al., 2013, BMC Cancer. 13:328.

In one embodiment of the methods or uses or product for uses described herein, response to treatment with tucatinib described and an anti-PD-L1 antibody described herein herein is assessed by measuring the time of progression free survival after administration of tucatinib described herein and/or anti-PD-L1 antibody described herein. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib described herein and/or anti-PD-L1 antibody described herein. In some embodiments, the subject exhibits progression-free survival of at least about 6 months after administration of tucatinib described herein and/or anti-PD-L1 antibody described herein. In some embodiments, the subject exhibits progression-free survival of at least about one year after administration of tucatinib described herein and/or anti-PD-L1 antibody described herein. In some embodiments, the subject exhibits progression-free survival of at least about two years after administration of tucatinib described herein and/or anti-PD-L1 antibody described herein. In some embodiments, the subject exhibits progression-free survival of at least about three years after administration of tucatinib described herein and/or anti-PD-L1 antibody described herein. In some embodiments, the subject exhibits progression-free survival of at least about four years after administration of tucatinib described herein and/or anti-PD-L1 antibody described herein. In some embodiments, the subject exhibits progression-free survival of at least about five years after administration of tucatinib described herein and/or anti-PD-L1 antibody described herein. In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of tucatinib described herein and/or anti-PD-L1 antibody described herein. In some embodiments, the subject exhibits progression-free survival of at least 6 months after administration of tucatinib described herein and/or anti-PD-L1 antibody described herein. In some embodiments, the subject exhibits progression-free survival of at least one year after administration of tucatinib described herein and/or anti-PD-L1 antibody described herein. In some embodiments, the subject exhibits progression-free survival of at least two years after administration of tucatinib described herein and/or anti-PD-L1 antibody described herein. In some embodiments, the subject exhibits progression-free survival of at least three years after administration of tucatinib described herein and/or anti-PD-L1 antibody described herein. In some embodiments, the subject exhibits progression-free survival of at least four years after administration of tucatinib described herein and/or anti-PD-L1 antibody described herein. In some embodiments, the subject exhibits progression-free survival of at least five years after administration of tucatinib described herein and/or anti-PD-L1 antibody described herein.

In one embodiment of the methods or uses or product for uses described herein, response to treatment with tucatinib described herein and an anti-PD-L1 antibody described herein is assessed by measuring the time of overall survival after administration of tucatinib described herein and/or anti-PD-L1 antibody described herein. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib described herein and/or anti-PD-L1 antibody described herein. In some embodiments, the subject exhibits overall survival of at least about 6 months after administration of tucatinib described herein and/or anti-PD-L1 antibody described herein. In some embodiments, the subject exhibits overall survival of at least about one year after administration of tucatinib described herein and/or anti-PD-L1 antibody described herein. In some embodiments, the subject exhibits overall survival of at least about two years after administration of tucatinib described herein and/or anti-PD-L1 antibody described herein. In some embodiments, the subject exhibits overall survival of at least about three years after administration of tucatinib described herein and/or anti-PD-L1 antibody described herein. In some embodiments, the subject exhibits overall survival of at least about four years after administration of tucatinib described herein and/or anti-PD-L1 antibody described herein. In some embodiments, the subject exhibits overall survival of at least about five years after administration of tucatinib described herein and/or anti-PD-L1 antibody described herein. In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least about 12 months, at least eighteen months, at least two years, at least three years, at least four years, or at least five years after administration of tucatinib described herein and/or anti-PD-L1 antibody described herein. In some embodiments, the subject exhibits overall survival of at least 6 months after administration of tucatinib described herein and/or anti-PD-L1 antibody described herein. In some embodiments, the subject exhibits overall survival of at least one year after administration of tucatinib described herein and/or anti-PD-L1 antibody described herein. In some embodiments, the subject exhibits overall survival of at least two years after administration of tucatinib described herein and/or anti-PD-L1 antibody described herein. In some embodiments, the subject exhibits overall survival of at least three years after administration of tucatinib described herein and/or anti-PD-L1 antibody described herein. In some embodiments, the subject exhibits overall survival of at least four years after administration of tucatinib described herein and/or anti-PD-L1 antibody described herein. In some embodiments, the subject exhibits overall survival of at least five years after administration of tucatinib described herein and/or anti-PD-L1 antibody described herein.

In one embodiment of the methods or uses or product for uses described herein, response to treatment with tucatinib described herein and an anti-PD-L1 antibody described herein is assessed by measuring the duration of response to tucatinib described herein and anti-PD-L1 antibody described herein after administration of tucatinib described herein and/or anti-PD-L1 antibody described herein. In some embodiments, the duration of response to tucatinib described herein and an anti-PD-L1 antibody described herein is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of tucatinib described herein and/or anti-PD-L1 antibody described herein. In some embodiments, the duration of response to tucatinib described herein and an anti-PD-L1 antibody described herein is at least about 6 months after administration of tucatinib described herein and/or anti-PD-L1 antibody described herein. In some embodiments, the duration of response to tucatinib described herein and an anti-PD-L1 antibody described herein is at least about one year after administration of tucatinib described herein and/or anti-PD-L1 antibody described herein. In some embodiments, the duration of response to tucatinib described herein and an anti-PD-L1 antibody described herein is at least about two years after administration of tucatinib described herein and/or anti-PD-L1 antibody described herein. In some embodiments, the duration of response to tucatinib described herein and an anti-PD-L1 antibody described herein is at least about three years after administration of tucatinib described herein and/or anti-PD-L1 antibody described herein. In some embodiments, the duration of response to tucatinib described herein and an anti-PD-L1 antibody described herein is at least about four years after administration of tucatinib described herein and/or anti-PD-L1 antibody described herein. In some embodiments, the duration of response to tucatinib described herein and an anti-PD-L1 antibody described herein is at least about five years after administration of tucatinib described herein and/or anti-PD-L1 antibody described herein. In some embodiments, the duration of response to tucatinib described herein and an anti-PD-L1 antibody described herein is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of tucatinib described herein and/or anti-PD-L1 antibody described herein. In some embodiments, the duration of response to tucatinib described herein and an anti-PD-L1 antibody described herein is at least 6 months after administration of tucatinib described herein and/or anti-PD-L1 antibody described herein. In some embodiments, the duration of response to tucatinib described herein and an anti-PD-L1 antibody described herein is at least one year after administration of tucatinib described herein and/or anti-PD-L1 antibody described herein. In some embodiments, the duration of response to tucatinib described herein and an anti-PD-L1 antibody described herein is at least two years after administration of tucatinib described herein and/or anti-PD-L1 antibody described herein. In some embodiments, the duration of response to tucatinib described herein and an anti-PD-L1 antibody described herein is at least three years after administration of tucatinib described herein and/or anti-PD-L1 antibody described herein. In some embodiments, the duration of response to tucatinib described herein and an anti-PD-L1 antibody described herein is at least four years after administration of tucatinib described herein and/or anti-PD-L1 antibody described herein. In some embodiments, the duration of response to tucatinib described herein and an anti-PD-L1 antibody described herein is at least five years after administration of tucatinib described herein and/or anti-PD-L1 antibody described herein.

H. Compositions

In another aspect, the present invention provides a pharmaceutical composition comprising tucatinib described herein and a pharmaceutically acceptable carrier. In another aspect, the present invention provides a pharmaceutical composition comprising an anti-PD-1 antibody described herein and a pharmaceutically acceptable carrier. In another aspect, the present invention provides a pharmaceutical composition comprising an anti-CTLA4 antibody described herein and a pharmaceutically acceptable carrier. In another aspect, the present invention provides a pharmaceutical composition comprising tucatinib described herein, an anti-PD-1 antibody described herein, and a pharmaceutically acceptable carrier. In some embodiments, the anti-PD-1 antibody is a member selected from the group consisting of pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some instances, the anti-PD-1 antibody is pembrolizumab. In some embodiments, the anti-PD-1 antibody is nivolumab. In some embodiments, the anti-CTLA4 antibody is ipilimumab.

In another aspect, the present invention provides a pharmaceutical composition comprising tucatinib described herein and a pharmaceutically acceptable carrier. In another aspect, the present invention provides a pharmaceutical composition comprising an anti-PD-L1 antibody described herein and a pharmaceutically acceptable carrier. In another aspect, the present invention provides a pharmaceutical composition comprising an anti-CTLA4 antibody described herein and a pharmaceutically acceptable carrier. In another aspect, the present invention provides a pharmaceutical composition comprising tucatinib described herein, an anti-PD-L1 antibody described herein, and a pharmaceutically acceptable carrier. In some embodiments, the anti-PD-L1 antibody is a member selected from the group consisting of atezolizumab, BMS-936559, durvalumab, avelumab, envafolimab, CK-301, CS-1001, SHR-1316, CBT-502, and BGB-A333. In some instances, the anti-PD-L1 antibody is atezolizumab. In some embodiments, the anti-PD-11 antibody is BMS-936559. In some instances, the anti-PD-L1 antibody is atezolizumab. In some embodiments, the anti-PD-11 antibody is durvalumab. In some instances, the anti-PD-L1 antibody is atezolizumab. In some embodiments, the anti-PD-11 antibody is avelumab. In some embodiments, the anti-CTLA4 antibody is ipilimumab.

In some embodiments, tucatinib described herein is present at a concentration between about 0.1 nM and 10 nM (e.g., about 0.1, 0.2, 0.3, 0.4, 0.5 0.6, 0.7, 0.8, 0.9, 1.0, 1.5, 2, 2.5, 3, 3.5, 4, 4.5, 5, 5.5, 6, 6.5, 7, 7.5, 8, 8.5, 9, 9.5, or 10 nM). In other embodiments, tucatinib described herein is present at a concentration between about 10 nM and 100 nM (e.g., about 10, 20, 25, 30, 35, 40, 45, 50, 55, 60, 65, 70, 75, 80, 85, 90, 95, or 100 nM). In some other embodiments, tucatinib described herein is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, tucatinib described herein is present at a concentration at least about 1,000 nM to 10,000 nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM).

In some embodiments, the anti-PD-1 antibody described herein is present at a concentration between about 0.1 nM and 10 nM (e.g., about 0.1, 0.2, 0.3, 0.4, 0.5 0.6, 0.7, 0.8, 1.0, 1.5, 2, 2.5, 3, 3.5, 4, 4.5, 5, 5.5, 6, 6.5, 7, 7.5, 8, 8.5, 9, 9.5, or 10 nM). In other embodiments, the anti-PD-1 antibody described herein is present at a concentration between about 10 nM and 100 nM (e.g., about 10, 15, 20, 25, 30, 35, 40, 45, 50, 55, 60, 65, 70, 75, 80, 85, 95, or 100 nM). In some other embodiments, the anti-PD-1 antibody is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, the anti-PD-1 antibody is present at a concentration of at least about 1,000 nM to nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM).

In some embodiments, the anti-PD-L1 antibody described herein is present at a concentration between about 0.1 nM and 10 nM (e.g., about 0.1, 0.2, 0.3, 0.4, 0.5 0.6, 0.7, 0.8, 1.0, 1.5, 2, 2.5, 3, 3.5, 4, 4.5, 5, 5.5, 6, 6.5, 7, 7.5, 8, 8.5, 9, 9.5, or 10 nM). In other embodiments, the anti-PD-L1 antibody described herein is present at a concentration between about 10 nM and 100 nM (e.g., about 10, 15, 20, 25, 30, 35, 40, 45, 50, 55, 60, 65, 70, 75, 80, 85, 95, or 100 nM). In some other embodiments, the anti-PD-L1 antibody is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, the anti-PD-L1 antibody is present at a concentration of at least about 1,000 nM to nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM).

In some embodiments, the anti-CTLA4 antibody described herein is present at a concentration between about 0.1 nM and 10 nM (e.g., about 0.1, 0.2, 0.3, 0.4, 0.5 0.6, 0.7, 0.8, 1.0, 1.5, 2, 2.5, 3, 3.5, 4, 4.5, 5, 5.5, 6, 6.5, 7, 7.5, 8, 8.5, 9, 9.5, or 10 nM). In other embodiments, the anti-CTLA4 antibody described herein is present at a concentration between about 10 nM and 100 nM (e.g., about 10, 15, 20, 25, 30, 35, 40, 45, 50, 55, 60, 65, 70, 75, 80, 85, 95, or 100 nM). In some other embodiments, the anti-CTLA4 antibody is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, the anti-CTLA4 antibody is present at a concentration of at least about 1,000 nM to 10,000 nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM).

The pharmaceutical compositions of the present invention may be prepared by any of the methods well-known in the art of pharmacy. Pharmaceutically acceptable carriers suitable for use with the present invention include any of the standard pharmaceutical carriers, buffers and excipients, including phosphate-buffered saline solution, water, and emulsions (such as an oil/water or water/oil emulsion), and various types of wetting agents or adjuvants. Suitable pharmaceutical carriers and their formulations are described in Remington's Pharmaceutical Sciences (Mack Publishing Co., Easton, 19th ed. 1995). Preferred pharmaceutical carriers depend upon the intended mode of administration of the active agent.

The pharmaceutical compositions of the present invention can include a combination of drugs (e.g., tucatinib described herein and/or an anti-PD-1 antibody described herein and/or an anti-PD-L1 antibody described herein and/or an anti-CTLA4 antibody described herein), or any pharmaceutically acceptable salts thereof, as active ingredients and a pharmaceutically acceptable carrier or excipient or diluent. A pharmaceutical composition may optionally contain other therapeutic ingredients.

The compositions (e.g., comprising tucatinib described herein, an anti-PD-1 antibody described herein, an anti-PD-L1 antibody described herein, an anti-CTLA4 antibody described herein, or a combination thereof) can be combined as the active ingredients in intimate admixture with a suitable pharmaceutical carrier or excipient according to conventional pharmaceutical compounding techniques. Any carrier or excipient suitable for the form of preparation desired for administration is contemplated for use with the compounds disclosed herein.

The pharmaceutical compositions include those suitable for oral, topical, parenteral, pulmonary, nasal, or rectal administration. The most suitable route of administration in any given case will depend in part on the nature and severity of the cancer condition and also optionally the HER2 status or stage of the cancer.

Other pharmaceutical compositions include those suitable for systemic (e.g., enteral or parenteral) administration. Systemic administration includes oral, rectal, sublingual, or sublabial administration. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In particular embodiments, pharmaceutical compositions of the present invention may be administered intratumorally.

Compositions for pulmonary administration include, but are not limited to, dry powder compositions consisting of the powder of a compound described herein (e.g., tucatinib described herein, an anti-PD-1 antibody described herein, an anti-PD-L1 antibody described herein, an anti-CTLA4 antibody described herein, or a combination thereof), or a salt thereof, and the powder of a suitable carrier or lubricant. The compositions for pulmonary administration can be inhaled from any suitable dry powder inhaler device known to a person skilled in the art.

Compositions for systemic administration include, but are not limited to, dry powder compositions consisting of the composition as set forth herein (e.g., tucatinib described herein, an anti-PD-1 antibody described herein, an anti-PD-L1 antibody described herein, an anti-CTLA4 antibody described herein, or a combination thereof) and the powder of a suitable carrier or excipient. The compositions for systemic administration can be represented by, but not limited to, tablets, capsules, pills, syrups, solutions, and suspensions.

In some embodiments, the compositions (e.g., tucatinib described herein, an anti-PD-1 antibody described herein, an anti-PD-L1 antibody described herein, an anti-CTLA4 antibody described herein, or a combination thereof) further include a pharmaceutical surfactant. In other embodiments, the compositions further include a cryoprotectant. In some embodiments, the cryoprotectant is selected from the group consisting of glucose, sucrose, trehalose, lactose, sodium glutamate, PVP, HPβCD, CD, glycerol, maltose, mannitol, and saccharose.

Pharmaceutical compositions or medicaments for use in the present invention can be formulated by standard techniques using one or more physiologically acceptable carriers or excipients. Suitable pharmaceutical carriers are described herein and in Remington: The Science and Practice of Pharmacy, 21st Ed., University of the Sciences in Philadelphia, Lippencott Williams & Wilkins (2005).

Controlled-release parenteral formulations of the compositions (e.g., tucatinib described herein, an anti-PD-1 antibody described herein, an anti-PD-L1 antibody described herein, an anti-CTLA4 antibody described herein, or a combination thereof) can be made as implants, oily injections, or as particulate systems. For a broad overview of delivery systems see Banga, A. J., Therapeutic Peptides and Proteins: Formulation, Processing, and Delivery Systems, Technomic Publishing Company, Inc., Lancaster, PA, (1995), which is incorporated herein by reference. Particulate systems include microspheres, microparticles, microcapsules, nanocapsules, nanospheres, and nanoparticles.

Polymers can be used for ion-controlled release of compositions of the present invention. Various degradable and nondegradable polymeric matrices for use in controlled drug delivery are known in the art (Langer R., Accounts Chem. Res., 26:537-542 (1993)). For example, the block copolymer, polaxamer 407 exists as a viscous yet mobile liquid at low temperatures but forms a semisolid gel at body temperature. It has been shown to be an effective vehicle for formulation and sustained delivery of recombinant interleukin 2 and urease (Johnston et al., Pharm. Res., 9:425-434 (1992); and Pec et al., J. Parent. Sci. Tech., 44(2):58 65 (1990)). Alternatively, hydroxyapatite has been used as a microcarrier for controlled release of proteins (Ijntema et al., Int. J. Pharm., 112:215-224 (1994)). In yet another aspect, liposomes are used for controlled release as well as drug targeting of the lipid-capsulated drug (Betageri et al., LIPOSOME DRUG DELIVERY SYSTEMS, Technomic Publishing Co., Inc., Lancaster, PA (1993)). Numerous additional systems for controlled delivery of therapeutic proteins are known. See, e.g., U.S. Pat. Nos. 5,055,303, 5,188,837, 4,235,871, 4,501,728, 4,837,028 4,957,735 and 5,055,303; 5,514,670; 5,413,797; 5,268,164; 5,004,697; 4,902,505; 5,506,206, 5,254,342 and 5,534,496, each of which is incorporated herein by reference.

For oral administration of a combination of tucatinib described herein and/or an anti-PD-1 antibody described herein and/or an anti-PD-L1 antibody described herein and/or an anti-CTLA4 antibody described herein, a pharmaceutical composition or a medicament can take the form of, for example, a tablet or a capsule prepared by conventional means with a pharmaceutically acceptable excipient. The present invention provides tablets and gelatin capsules comprising tucatinib described herein, an anti-PD-1 antibody described herein, an anti-PD-L1 antibody described herein, an anti-CTLA4 antibody described herein, or a combination thereof, or a dried solid powder of these drugs, together with (a) diluents or fillers, e.g., lactose, dextrose, sucrose, mannitol, sorbitol, cellulose (e.g., ethyl cellulose, microcrystalline cellulose), glycine, pectin, polyacrylates or calcium hydrogen phosphate, calcium sulfate, (b) lubricants, e.g., silica, talcum, stearic acid, magnesium or calcium salt, metallic stearates, colloidal silicon dioxide, hydrogenated vegetable oil, corn starch, sodium benzoate, sodium acetate or polyethyleneglycol; for tablets also (c) binders, e.g., magnesium aluminum silicate, starch paste, gelatin, tragacanth, methylcellulose, sodium carboxymethylcellulose, polyvinylpyrrolidone or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, or sweetening agents as appropriate. If desired, preparations for oral administration can be suitably formulated to give controlled release of the active compound(s).

Typical formulations for topical administration of tucatinib described herein, an anti-PD-1 antibody described herein, an anti-PD-L1 antibody described herein, an anti-CTLA4 antibody described herein, or a combination thereof include creams, ointments, sprays, lotions, and patches. The pharmaceutical composition can, however, be formulated for any type of administration, e.g., intradermal, subdermal, intravenous, intramuscular, subcutaneous, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral or rectal administration is also contemplated.

Suitable formulations for transdermal application include an effective amount of one or more compounds described herein, optionally with a carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

The compositions and formulations set forth herein (e.g., tucatinib described herein, an anti-PD-1 antibody described herein, an anti-PD-L1 antibody described herein, an anti-CTLA4 antibody described herein, or a combination thereof) can be formulated for parenteral administration by injection, for example by bolus injection or continuous infusion. Formulations for injection can be presented in unit dosage form, for example, in ampules or in multi-dose containers, with an added preservative. Injectable compositions are preferably aqueous isotonic solutions or suspensions, and suppositories are preferably prepared from fatty emulsions or suspensions. The compositions may be sterilized or contain adjuvants, such as preserving, stabilizing, wetting or emulsifying agents, solution promoters, salts for regulating the osmotic pressure or buffers. Alternatively, the active ingredient(s) can be in powder form for constitution with a suitable vehicle, for example, sterile pyrogen-free water, before use. In addition, they may also contain other therapeutically valuable substances. The compositions are prepared according to conventional mixing, granulating or coating methods, respectively.

For administration by inhalation, the compositions (e.g., comprising tucatinib described herein, an anti-PD-1 antibody described herein, an anti-PD-L1 antibody described herein, an anti-CTLA4 antibody described herein, or a combination thereof) may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

The compositions (e.g., comprising tucatinib described herein, an anti-PD-1 antibody described herein, an anti-PD-L1 antibody described herein, an anti-CTLA4 antibody described herein, or a combination thereof) can also be formulated in rectal compositions, for example, suppositories or retention enemas, for example, containing conventional suppository bases, for example, cocoa butter or other glycerides.

Furthermore, the active ingredient(s) can be formulated as a depot preparation. Such long-acting formulations can be administered by implantation (for example, subcutaneously or intramuscularly) or by intramuscular injection. Thus, for example, one or more of the compounds described herein can be formulated with suitable polymeric or hydrophobic materials (for example as an emulsion in an acceptable oil) or ion exchange resins, or as sparingly soluble derivatives, for example, as a sparingly soluble salt.

I. Articles of Manufacture and Kits

In another aspect, the present invention provides an article of manufacture or kit for treating or ameliorating the effects of a solid tumor in a subject, the article of manufacture or kit comprising a pharmaceutical composition of the present invention (e.g., a pharmaceutical composition comprising tucatinib described herein, an anti-PD-1 antibody described herein, an anti-PD-L1 antibody described herein, an anti-CTLA4 antibody as described herein, or a combination thereof). In some embodiments, the anti-PD-1 antibody is pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 or CS1003. In some instances, the anti-PD-1 antibody is pembrolizumab. In some embodiments, the anti-PD-1 antibody is nivolumab. In some embodiments, the anti-PD-L1 antibody is atezolizumab, BMS-936559, durvalumab, avelumab, envafolimab, CK-301, CS-1001, SHR-1316, CBT-502, or BGB-A333. In some embodiments, the anti-PD-L1 antibody is atezolizumab. In some embodiments, the anti-PD-L1 antibody is BMS-936559. In some embodiments, the anti-PD-L1 antibody is durvalumab. In some embodiments, the anti-PD-L1 antibody is avelumab. In some instances, the anti-CTLA4 antibody is ipilimumab.

The articles of manufacture or kits are suitable for treating or ameliorating the effects of cancers, particularly solid tumors. In some embodiments, the cancer is an advanced cancer.

Materials and reagents to carry out the various methods of the present invention can be provided in articles of manufacture or kits to facilitate execution of the methods. As used herein, the term “kit” includes a combination of articles that facilitates a process, assay, analysis, or manipulation. In particular, kits of the present invention find utility in a wide range of applications including, for example, diagnostics, prognostics, therapy, and the like.

Articles of manufacture or kits can contain chemical reagents as well as other components. In addition, the articles of manufacture or kits of the present invention can include, without limitation, instructions to the user, apparatus and reagents for administering combinations of tucatinib described herein and anti-PD-1 antibodies described herein or pharmaceutical compositions thereof, sample tubes, holders, trays, racks, dishes, plates, solutions, buffers, or other chemical reagents. In addition, the articles of manufacture or kits of the present invention can include, without limitation, instructions to the user, apparatus and reagents for administering combinations of tucatinib described herein and anti-PD-L1 antibodies described herein or pharmaceutical compositions thereof, sample tubes, holders, trays, racks, dishes, plates, solutions, buffers, or other chemical reagents. In some embodiments, the articles of manufacture or kits contain instructions, apparatus, or reagents for determining the genotype of a gene (e.g., HER2, KRAS, NRAS, BRAF) or determining the expression of HER2 in a sample. Articles of manufacture or kits of the present invention can also be packaged for convenient storage and safe shipping, for example, in a box having a lid.

### III. Binding Assays and Other Assays

In one aspect, an antibody of the invention is tested for its antigen binding activity, for example, by known methods such as Enzyme-Linked Immunosorbant Assay (ELISA), immunoblotting (e.g., Western blotting), flow cytometry (e.g., FACS™), immunohistochemistry, immunofluorescence, etc.

In another aspect, competition assays may be used to identify an antibody that competes with any one of the antibodies described herein for binding to PD-1, PD-L1, or CTLA4 Cross-competing antibodies can be readily identified based on their ability to cross-compete in standard PD-1, PD-L1, or CTLA4 binding assays such as Biacore analysis, ELISA assays or flow cytometry (See, e.g., WO 2013/173223). In certain embodiments, such a competing antibody binds to the same epitope (e.g., a linear or a conformational epitope) that is bound by any one of the antibodies disclosed herein. Detailed exemplary methods for mapping an epitope to which an antibody binds are provided in Morris “Epitope Mapping Protocols,” in Methods in Molecular Biology Vol. 66 (Humana Press, Totowa, N J, 1996).

In an exemplary competition assay, immobilized PD-1 is incubated in a solution comprising a first labeled antibody that binds to PD-1 and a second unlabeled antibody that is being tested for its ability to compete with the first antibody for binding to PD-1. The second antibody may be present in a hybridoma supernatant. As a control, immobilized PD-1 is incubated in a solution comprising the first labeled antibody but not the second unlabeled antibody. After incubation under conditions permissive for binding of the first antibody to PD-1, excess unbound antibody is removed, and the amount of label associated with immobilized PD-1 is measured. If the amount of label associated with immobilized PD-1 is substantially reduced in the test sample relative to the control sample, then that indicates that the second antibody is competing with the first antibody for binding to PD-1. See, e.g., Harlow et al. Antibodies: A Laboratory Manual. Ch. 14 (Cold Spring Harbor Laboratory, Cold Spring Harbor, N Y, 1988). In some embodiments, an anti-PD-1 antibody competes for binding to PD-1 with another PD-1 antibody (e.g., pembrolizumab) if the antibody blocks binding of the other antibody to PD-1 in a competition assay by more than 20%, more than 25%, more than 30%, more than 35%, more than 40%, more than 45%, more than 50%, more than 55%, more than 60%, more than 65%, more than 70%, more than 75%, more than 80%, more than 85%, more than 90%, more than 95%. In some embodiments, an anti-PD-1 antibody does not compete for binding to PD-1 with another PD-1 antibody (e.g., pembrolizumab) if the antibody blocks binding of the other antibody to PD-1 in a competition assay by less than 20%, less than 15%, less than 10%, less than 9%, less than 8%, less than 7%, less than 6%, less than 5%, less than 4%, less than 3%, less than 2%, less than 1%. In some embodiments, the PD-1 is human PD-1.

Similar competition assays can be performed to determine if an anti-PD-L1 antibody competes with an anti-PD-L1 antibody described herein for binding to PD-L1. In some embodiments, an anti-PD-L1 antibody competes for binding to PD-L1 with another PD-L1 antibody if the antibody blocks binding of the other antibody to PD-L1 in a competition assay by more than 20%, more than 25%, more than 30%, more than 35%, more than 40%, more than 45%, more than 50%, more than 55%, more than 60%, more than 65%, more than 70%, more than 75%, more than 80%, more than 85%, more than 90%, more than 95%. In some embodiments, an anti-PD-L1 antibody does not compete for binding to PD-L1 with another PD-L1 antibody if the antibody blocks binding of the other antibody to PD-L1 in a competition assay by less than 20%, less than 15%, less than 10%, less than 9%, less than 8%, less than 7%, less than 6%, less than 5%, less than 4%, less than 3%, less than 2%, less than 1%. In some embodiments, the PD-L1 is human PD-L1.

Similar competition assays can be performed to determine if an anti-CTLA4 antibody competes with an anti-CTLA4 antibody described herein for binding to CTLA4. In some embodiments, an anti-CTLA4 antibody competes for binding to CTLA4 with another CTLA4 antibody if the antibody blocks binding of the other antibody to CTLA4 in a competition assay by more than 20%, more than 25%, more than 30%, more than 35%, more than 40%, more than 45%, more than 50%, more than 55%, more than 60%, more than 65%, more than 70%, more than 75%, more than 80%, more than 85%, more than 90%, more than 95%. In some embodiments, an anti-CTLA4 antibody does not compete for binding to CTLA4 with another CTLA4 antibody if the antibody blocks binding of the other antibody to CTLA4 in a competition assay by less than 20%, less than 15%, less than 10%, less than 9%, less than 8%, less than 7%, less than 6%, less than 5%, less than 4%, less than 3%, less than 2%, less than 1%. In some embodiments, the CTLA4 is human CTLA4.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1: Effect of Tucatinib on the Tumor Microenvironment Ex Vivo

This study was designed to assess the effect of tucatinib on the tumor microenvironment ex vivo using trastuzumab sensitive and trastuzumab resistant murine HER2-positive tumor models. The trastuzumab sensitive model utilized H2N113 tumors administered by subcutaneous injection in female MMTV-Balb/c mice. The trastuzumab resistant model utilized Fo5 tumors implanted as mammary fat pad implants in FVB mice.

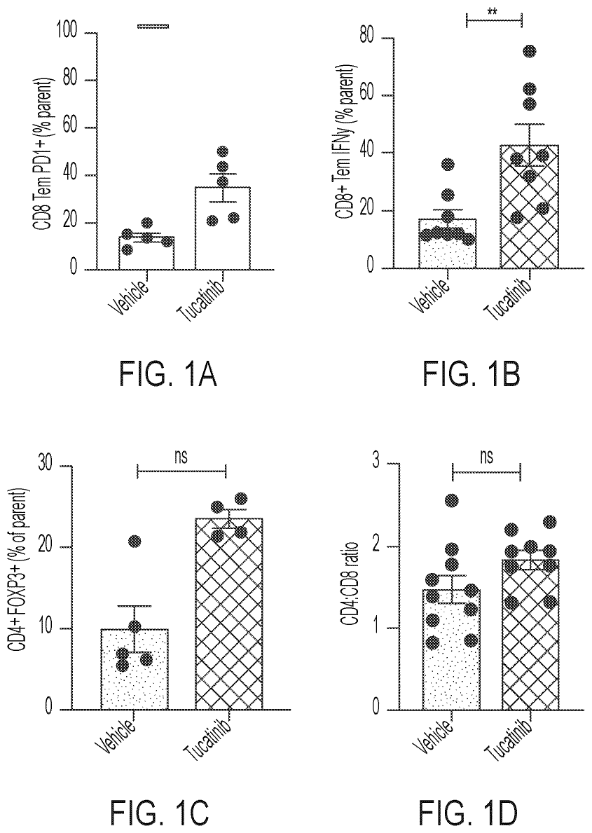

For the trastuzumab resistant model, mice were treated with vehicle control (methyl cellulose 0.5%) or tucatinib 50 mg/kg orally once daily. Tumors were harvested after 10-14 days of tucatinib 50 mg/kg treatment for Fo5 tumors and analyzed by FACS. As shown in FIG. 1A and FIG. 1B, tucatinib resulted in an increase in infiltration of CD8+ T cells expressing PD-1, and IFNγ in the tumor, respectively. As shown in FIG. 1C, tucatinib also resulted in an increase in infiltration of CD8+ T cells expressing FOXP3 in the tumor. Finally, as shown in FIG. 1D, tucatinib resulted in a moderate increase in the CD4+ to CD8+ T cell ratio in the tumor.

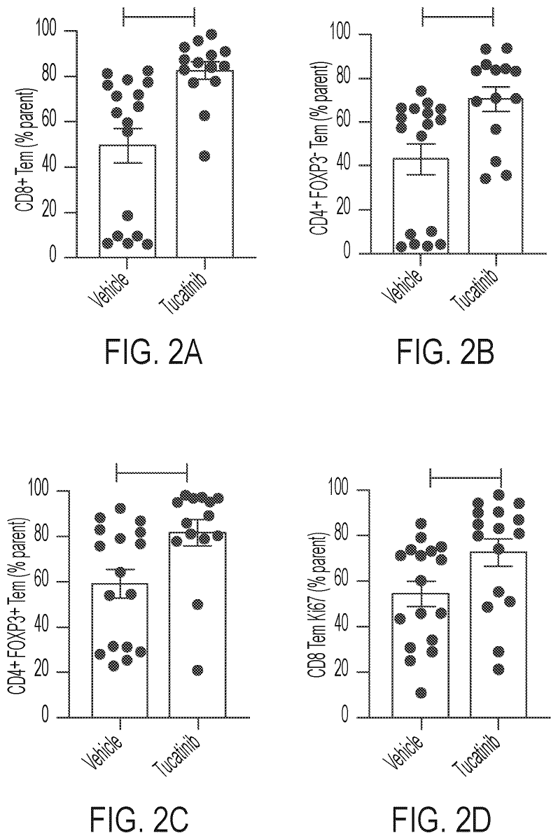

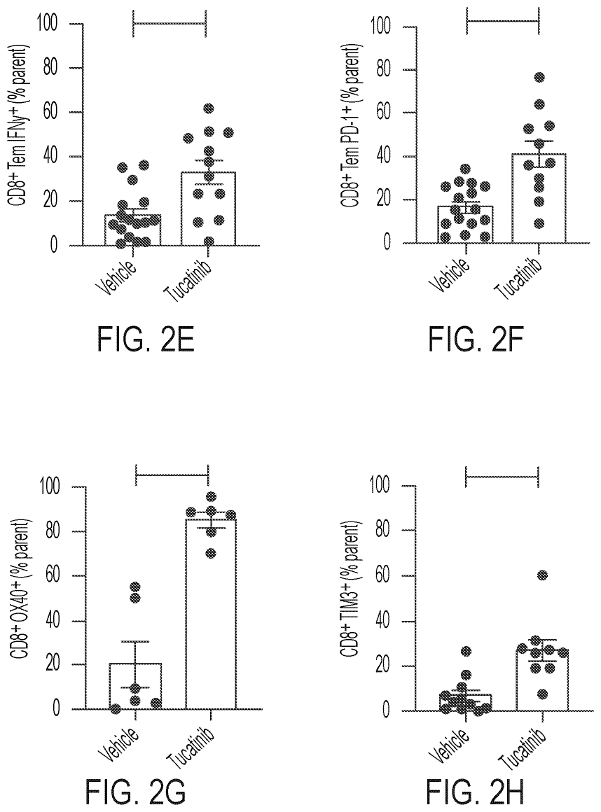

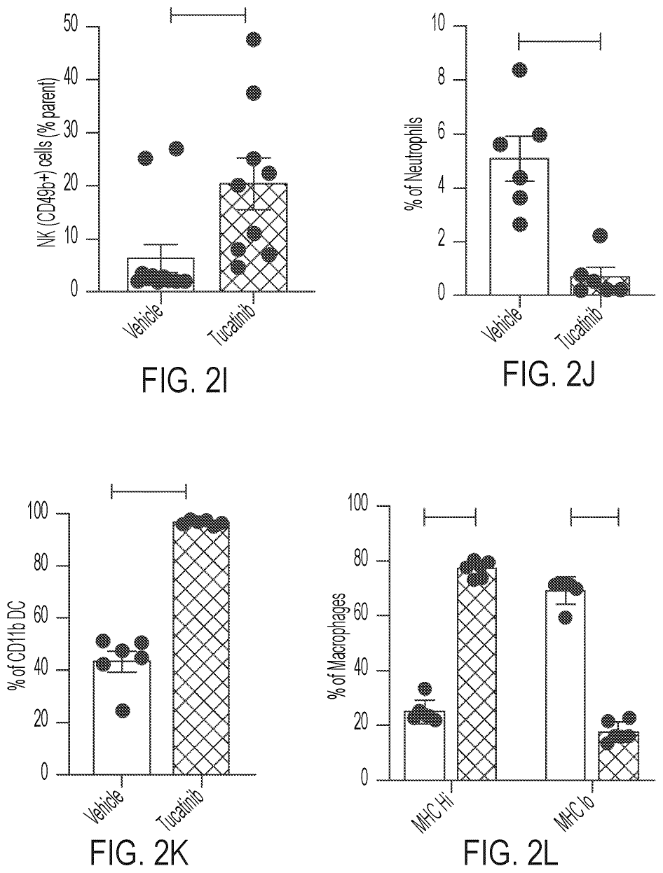

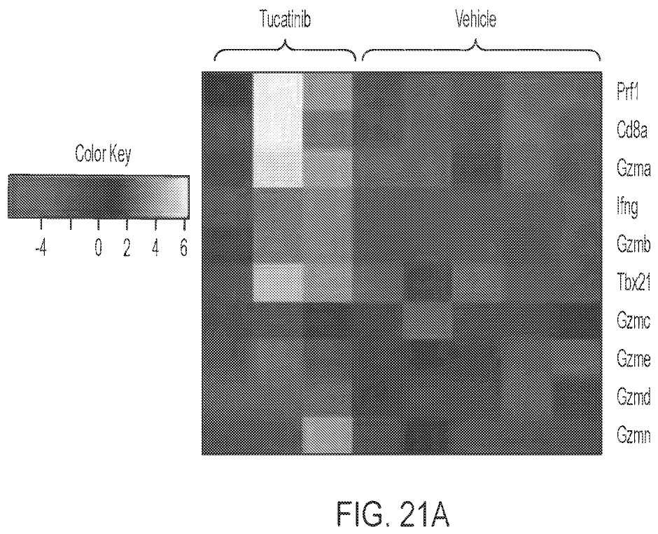

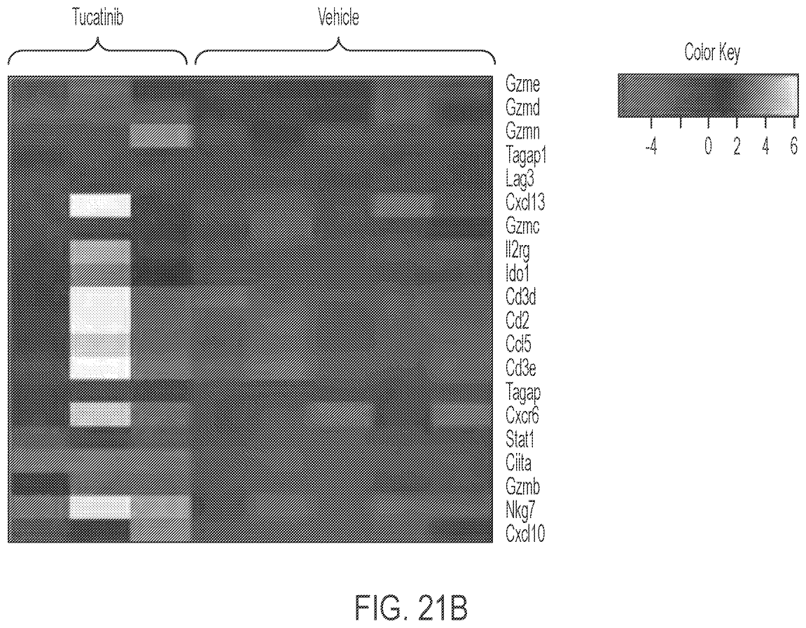

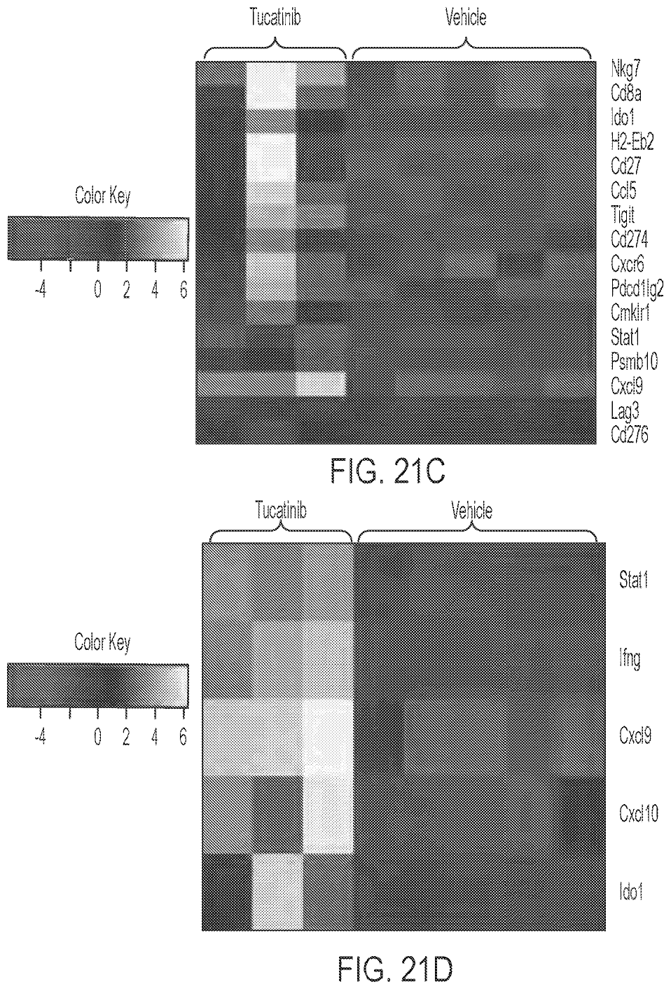

For the trastuzumab sensitive model, mice were treated with vehicle control (methyl cellulose 0.5%) or tucatinib 100 mg/kg orally once daily. Tumors were harvested after 14 days of tucatinib 100 mg/kg treatment for H2N113 tumors and analyzed by FACS. As shown in FIG. 2A, tucatinib resulted in an increase in the infiltration of CD8+ T cells in the tumor. As shown in FIG. 2B and FIG. 2C, tucatinib resulted in an increase in infiltration of CD4+ T cells not expressing FOXP3 and expressing FOXP3 in the tumor, respectively. As shown in FIG. 2D-2H, tucatinib also resulted in an increase in infiltration of CD8+ T cells expressing Ki67, IFNγ, PD-1, OX40, and TIM3 in the tumor, respectively. As shown in FIG. 21 tucatinib also resulted in an increase in infiltration of natural killer (NK) cells in the tumor. As shown in FIG. 2J, tucatinib resulted in a decrease in the infiltration of neutrophils in the tumor. As shown in FIG. 2K, tucatinib resulted in an increase in the percentage of CD11 b dendritic cells in the tumor. As shown in FIG. 2L, tucatinib resulted in an increase in the percentage of MHC-II high and a decrease in the percentage of MHC-II low in the tumor. These results suggest an increase in anti-tumor immunity following administration of tucatinib.

### Example 2: Effect of Tucatinib on the RNA Expression Profile of Tumor Samples Ex Vivo

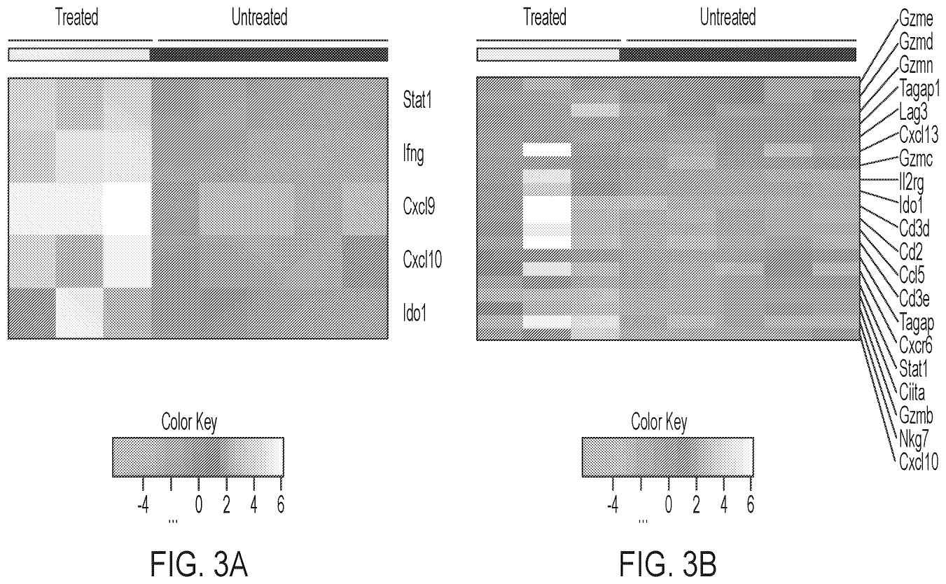

Trastuzumab resistant Fo5 were implanted, the mice were treated with vehicle control or tucatinib, and harvested as described in Example 1. The harvested tumors were then processed for stranded polyA RNA sequencing. As shown in FIGS. 3A and 3B, Fo5 tumors showed increased expression of genes that are predictive of response to immunotherapy as per an IFNγ-related gene expression signature (FIG. 3A) and expanded immune signature (FIG. 3B). Columns represent individual tumor samples and rows are individual genes.

### Example 3: Effect of Tucatinib on Tumor Growth and Survival In Vivo

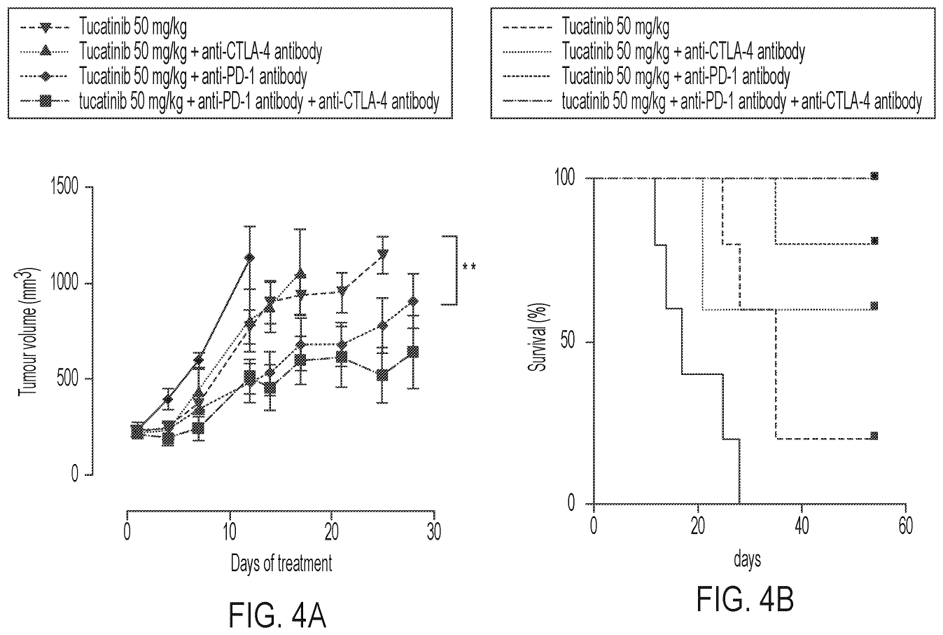

Trastuzumab resistant Fo5 tumors were implanted as mammary fat pad implants in FVB mice. Mice were treated with vehicle control (methyl cellulose 0.5%), tucatinib (25 mg/kg, 50 mg/kg, or 100 mg/kg orally once daily, or a combination of anti-PD-1 antibody, anti-CTLA4, or isotype 2A3. Tucatinib significantly inhibited tumor growth in a dose-dependent manner and was observed at 25 mg/kg, 50 mg/kg and 100 mg/kg (p<0.05). Similar results were obtained with the H2N113 trastuzumab sensitive tumor model. Furthermore, 50 mg/kg tucatinib in combination with PD-1 inhibition demonstrated significantly greater anti-tumor efficacy compared to tucatinib alone (FIG. 4A, p=0.0079) and increased survival (FIG. 4B, p=0.05).

### Example 4: Tucatinib Promotes Immune Activation and Synergies with PD-1/PD-L1 Inhibition in HER2+ Positive Breast Cancer

Cell lines and mouse patient-derived xenograft model. The murine cell line, H2N113, was grown in RPMI media with 10% FBS. This HER2-positive murine cell line was generated from transgenic female mice of BALB/C MMTV ErbB2/Neu background. Fo5 (MMTV-human HER2) tumors were obtained. Two human cell lines overexpressing HER2 were used for in vitro experiments: BT474 (ATCC Catalog Number: HTB-20) and SKBR3 (ATCC Catalog Number: HTB-30). BT474 cells are estrogen and progesterone receptor positive and HER2 amplified and maintained in RPMI media with 10% FBS. SKBR3 cells are estrogen and progesterone receptor negative and HER2 amplified and maintained in DMEM media with 10% FBS.

Drugs. For in vitro experiments, tucatinib was solubilized in DMSO at a concentration of 10 mM. For in vivo study, tucatinib was dissolved in 0.5% methylcellulose at the required concentration and stored at 4° C. for up to two months. Tucatinib was administered in vivo once daily by oral gavage at doses of 25, 50, or 100 mg/kg, as specified. Trastuzumab was stored at 4° C. and diluted in sterile PBS and administered via intraperitoneal injection with a loading dose of 30 mg/kg then 15 mg/kg weekly. Mouse specific antibodies for anti-PD-1, anti-PD-L1, and isotype controls (2A3, LTF2) were purchased from BioXcell and administered at 200 μg per mouse, twice weekly via intraperitoneal injection for two weeks. All antibodies were diluted in sterile PBS for in vivo studies.

Growth inhibition assays. To obtain GI₅₀ values for tucatinib for each cell line, the viability of cells was assessed across a wide range of doses. Cells were plated in 96-well white-walled plates and treated with logarithmically escalating doses of tucatinib. Cells were incubated at 37° C. for 72 hours and cell viability was determined based on quantitation of CellTitre-Glo® Assay (Promega) at 1:3 dilution and luminescence read using Cytation™ (BioTek). Dose-response curves were calculated using GraphPad Prism version 8.1.0 for macOS. All experiments were performed in at least triplicate, with a mean value used for GIs° calculations.

CFSE proliferation assay. Humans PBMCs were isolated and rested overnight in RPMI and 10% FBS. Cells were then stained with CFSE per manufacturer's protocol (Thermo Fisher) and 1×10⁵ cells were seeded in 96-well plates containing anti-CD3 (OKT3; 1:1000)/anti-CD28 beads (Thermo Fisher), with or without tucatinib at incremental doses (zero to 4 μM). After 96 hours, cells were stained with anti-CD3 (UCTH1; 500 ng/mL), anti-CD4 (clone OKT4, Biolegend; 500 ng/mL) and anti-CD8a (clone HIT8a, Biolegend; 500 ng/mL). Population doubling for CD4 and CD8 T cells was determined by integrating CFSE+ peaks using stained, unstimulated cells as a control. Analysis was performed using FlowJo™ software.

Western blot analysis. For Western blot analysis, CD8+ and CD4+ T cells were isolated from human PBMCs. 5×10⁶ T cells per condition were added to anti-CD3 (1 μg/mL) precoated 6-well plates and treated with either anti-CD28 (0.5 μg/mL), anti-CD28, and tucatinib at 10 nM or anti-CD28 and tucatinib at 1.6 μM. Pellets were lysed after 15 minutes or 1 hour of treatment using radioimmunoprecipitation assay (RIPA) buffer. Primary antibodies used for immunoblotting were purchased from Cell Signaling Technologies (Danvers, MA, USA) or Invitrogen (Thermo Fisher Scientific, Waltham, MA, USA): p-ERK1/2 (Thr202/Tyr204; #9101, 1:1000, 42/44 kDa), p-AKT (Ser473; D9E #4060, 1:1000, 65 kDa), p-PI3K (Tyr458/Tyr199; #4228, 1:1000, 85/55 kDa), p-ZAP70, p-ITK (Tyr512, #PA5-64523, 1:1000, 72 kDa), p-Src (Tyr416, #2101, 1:1000, 60 kDa), p-Lck (Tyr505, #2752, 1:1000, 56 kDa) and GAPDH loading control (ab-9484, 1:10,000, 40 kDa; Abcam, Cambridge, UK). HER2-positive human cell lines BT474 and SKBR3 and murine cell line H2N113 were treated with tucatinib (7 nM, 35 nM or 50 nM, respectively) for 1, 3, 6, 24 and 72 hours. Secondary antibody used was α-rabbit HRP IgG (Santa Cruz; sc-2005, 1:3000) for chemiluminescent signal detection.

Genomic analysis of mouse samples. RNA was extracted from Fo5 tumors treated in vivo with tucatinib at 50 mg/kg or vehicle (methyl cellulose, 0.5%) for 10 days using TRIzol® Reagent with the PureLink® RNA Mini Kit (Invitrogen™, Thermo Fisher Scientific, Waltham, MA, USA). Quantity and integrity of the total RNA was checked using a TapeStation 2200 (Agilent Technologies) and 500 ng used for library preparation according to standard protocols (NEBNext Ultra II Directional RNA Library Prep Kit for Illumina and NEBNext Poly(A) mRNA Magnetic Isolation Module, NEB). Indexed libraries were pooled and sequenced on a NextSeq500 flowcell (Illumina) to generate 25-50 million paired-end 75 bp reads per sample. Pre-processing and quantile normalization of microarray data was performed with the AFFY package in R. From normalized RNA intensities, unbiased, differential expression analysis and a short list of gene (FDR<0.05) was generated with the LIMMA package in R. Using the differentially expressed genes, pathway and molecular function enrichment analysis was performed in MetCore GeneCo. The GSEA software for gene set enrichment analysis was used with normalized RNA intensities. The false discovery rate was set at <10%. Top gene pathways were selected based on a P-value of <0.05 and a fold change of >1.5. All analyses were performed using R version 3.5.0. Gene sets for differential expression heat maps included interferon-γ 10-gene, preliminary expanded immune 28-gene, T cell inflamed 18-gene, and T-effector 6-gene signature sets, all of which have been associated with PD-1 or PD-L1 blockade amongst a range of tumor types, including breast cancer.

In vivo mouse studies. Female MMTV/Balb/c and FVB mice between 6 to 8 weeks of age were utilized for experiments. Treatment groups consisted of n=5 to 8 mice per group. For in vivo experiments using the H2N113 HER2-positive, trastuzumab sensitive tumor model, 5×10⁵ cells were suspended in phosphate-buffered saline and injected as subcutaneous single cell suspensions in 100 μL volume into the right flank of female MMTV-Balb/c mice. For in vivo experiments using the Fo5 HER2-positive trastuzumab resistant tumor model, tumor fragments of approximately 1 mm 3 were surgically implanted into the fourth mammary fat pad of female FVB mice. Randomization and treatment commenced at approximately day 10 post-inoculation for the H2N113 tumor model with tumor sizes varying between 40 to 80 mm³ and approximately day 14 for the Fo5 tumor model with tumor sizes varying between 60 to 120 mm³. Tumor volumes were calculated using the equation (length×width)/2, where length and width refer to the larger and smaller dimensions collected at each measurement. Following establishment of tumors and randomization into treatment groups, mice were treated with (1) vehicle control (methyl cellulose 0.5%), (2) tucatinib (25, 50, or 100 mg/kg orally, once daily), (3) trastuzumab alone (30 mg/kg loading dose, then 15 mg/kg weekly, intraperitoneal injection), (4) anti-PD-1 alone (200 μg, intraperitoneal injection), (5) isotype 2A3 alone (200 μg, intraperitoneal injection), (6) anti-PD-L1 alone (200 μg, intraperitoneal injection), (7) isotype LTF2 alone (200 intraperitoneal injection), or (8) double combination of tucatinib and either trastuzumab, anti-PD-1, isotype 2A3, anti-PD-1, or isotype LTF2. Immunotherapy and isotype controls in both models were delivered on days 0, 4, 8, and 12. Tumor volumes were measured twice weekly with calipers. Survival was monitored and determined when the tumors reached an ethical limit of 1500 mm³.

Flow cytometry analysis. For ex vivo studies, tumors were dissected, mechanically morcellated and passed through 70 μm filters. Cells were then resuspended in 30 μg/mL DNAse (Sigma-Aldrich) followed by Fc block (clone 2.4g2). Single cell suspensions were then stained with antibody cocktails for various TIL subsets. In some experiments, isolated cells were stimulated with phorbol 12-myristate 13-acetate (PMA; 50 ng/mL) and ionomycin (1 μg/mL; Sigma-Aldrich) in the presence of GolgiPlug (BD Biosciences; 1:1000) and GolgiStop (BD Biosciences; 1:1500) for 3 to 4 hours prior to flow cytometry analysis. Samples were analyzed by FACS with either Fixable Yellow or Zombie Red to discriminate viable and dead cells. BD fluorosphere counting beads were added to cocktails before running sample.

Statistical analysis. All statistical analyses were performed using Prism (version 9, GraphPad Software, Inc.) Data are presented +/−SEM.

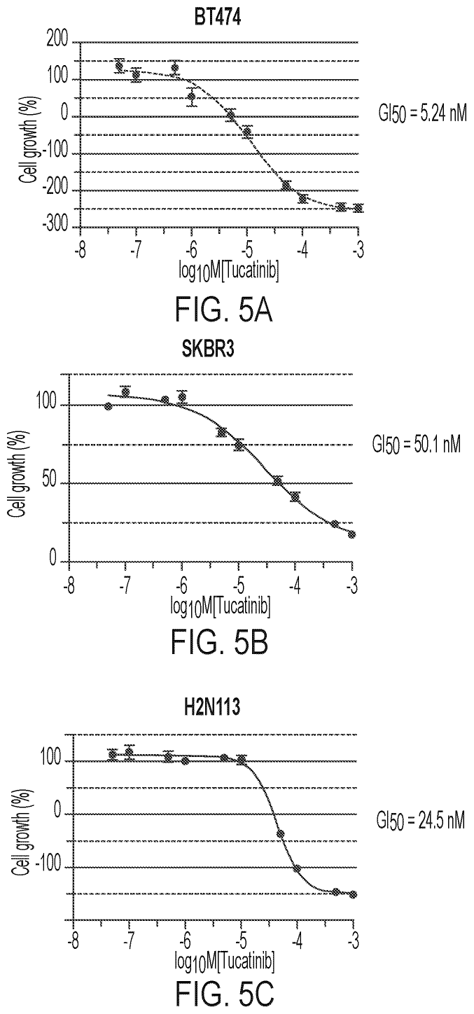

Results. As shown in FIG. 5A-5C, in HER2-positive human (BT474, FIG. 5A and SKBR3, FIG. 5B) and murine (H2N113, FIG. 5C) trastuzumab-sensitive breast tumor cell lines in vitro, tucatinib demonstrated dose-dependent cell growth inhibition with a GI₅₀ at 72 hours in the nanomolar range (data shown in FIG. 5A-5C are +/−SEM).

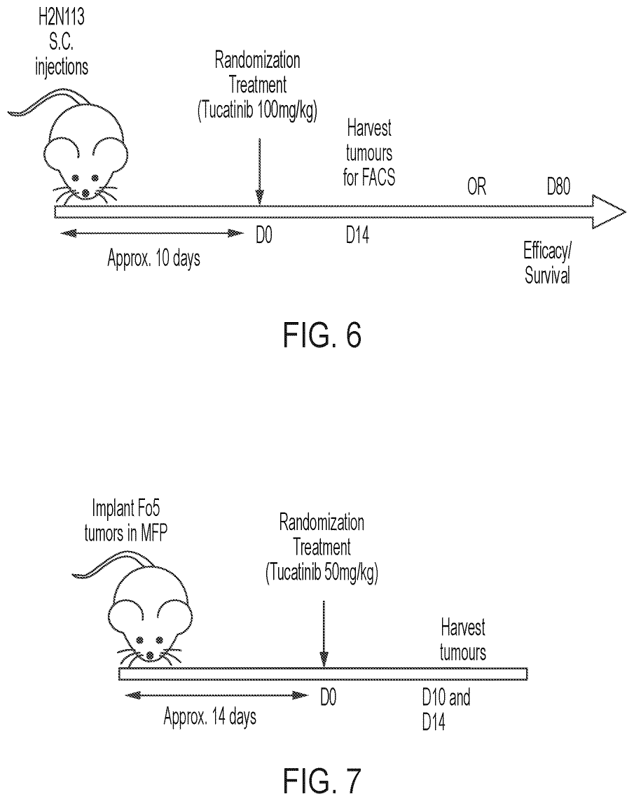

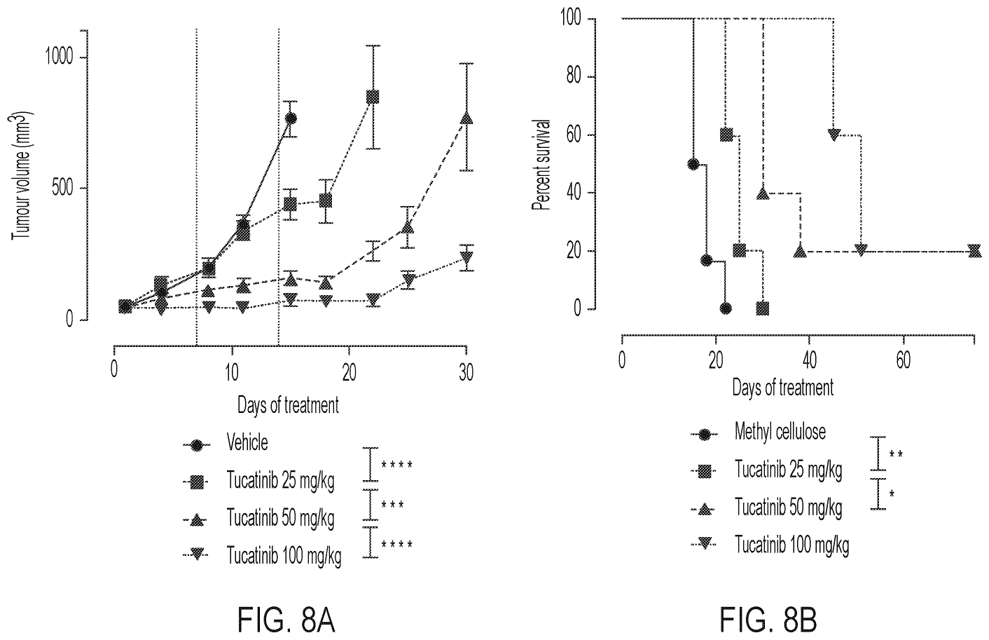

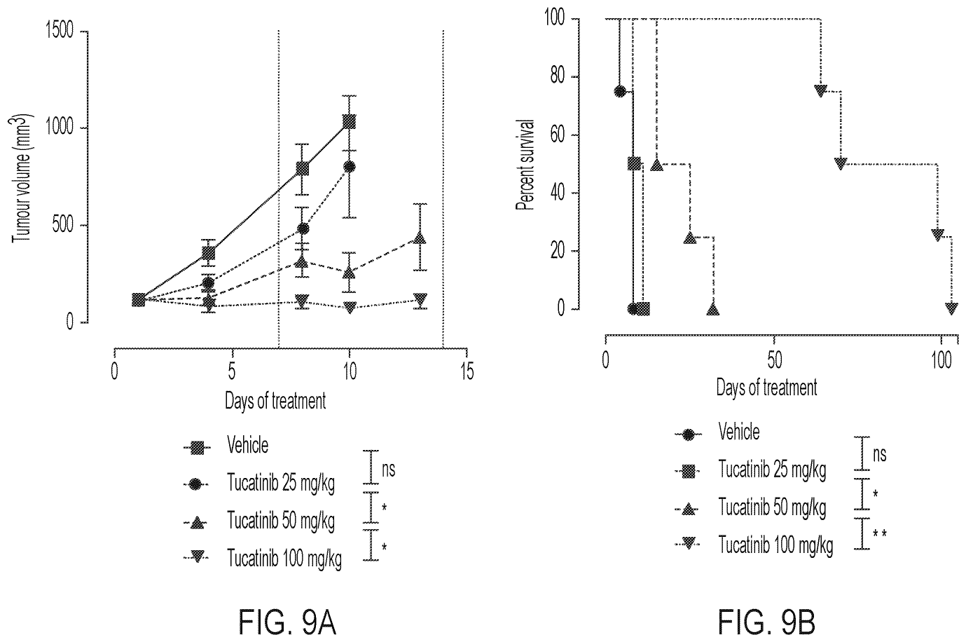

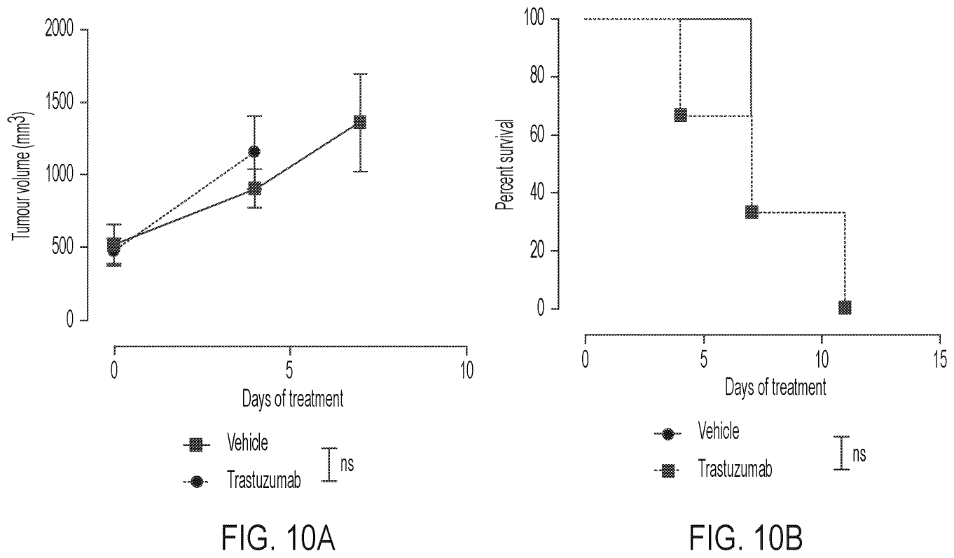

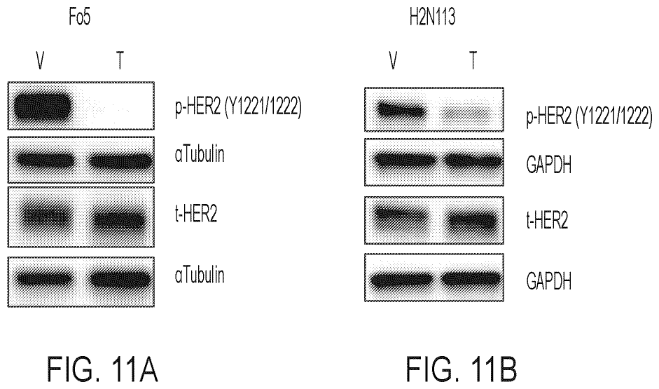

FIG. 6 shows the H2N113 trastuzumab-sensitive HER2-positive murine tumor model for assessing tucatinib effect. FIG. 7 shows the Fo5 trastuzumab-resistant HER2-positive murine tumor model for assessing tucatinib effect. Tucatinib treatment at increasing doses resulted in significant dose-dependent tumor growth inhibition and improved survival compared with vehicle in both model systems. Results for H2N113 mouse model are shown in FIG. 8A-8B. Results for Fo5 mouse model are shown in FIG. 9A-9B. In both models, tucatinib treatment resulted in a dose-dependent improvement in tumor volume and survival. As shown in FIG. 10A-10B, trastuzumab resistance was validated in the Fo5 model and confirms this system represents a clinically-relevant tumor model for the use of tucatinib in a trastuzumab resistant setting. Inhibition of the HER2 signaling pathway with tucatinib was confirmed using Western blot analysis for phosphor-HER2 and total HER2 in H2N113 cells and Fo5 tumors treated with tucatinib (FIG. 11A-11B; T=tucatinib, V=vehicle).

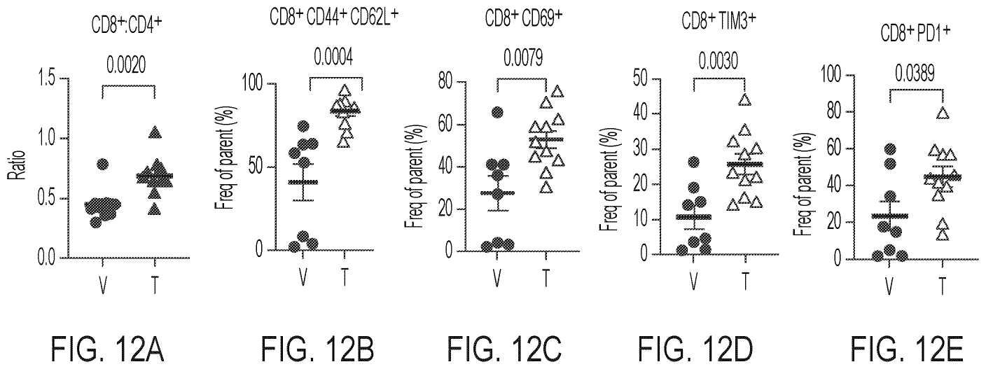

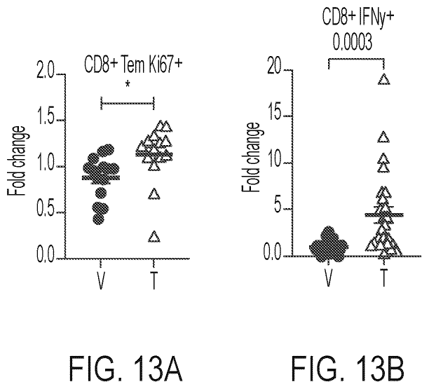

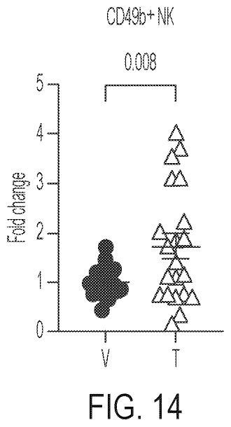

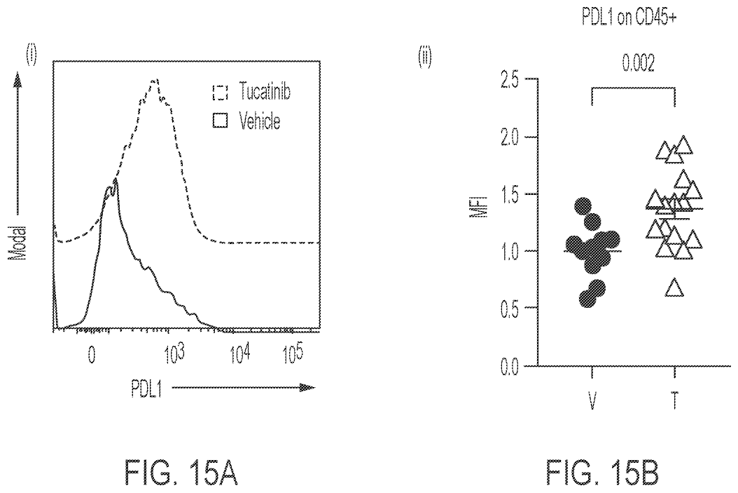

Extensive ex vivo FACS analysis of both trastuzumab sensitive and trastuzumab resistant tumor models was performed. H2N113 tumors after 14 days of tucatinib treatment demonstrated increased CD8+ proportion and frequency of CD8+ effector memory (CD44+CD62L−) T cell frequency as a percentage of total CD8+. CD8+ effector memory T cells showed significantly higher PD-1+ and TIM3+ expression. FIG. 12A-12E shows effect on various cell populations after treatment with vehicle control (V) or tucatinib (T) for H2N113 cells. These cells also exhibited increased expression of Ki67 (FIG. 13A, indicating increased cell proliferation, as well as increased effector function evidenced by significantly greater interferon-7 expression compared to vehicle treated controls (FIG. 13B). In addition, tucatinib treatment also resulted in a significantly increased CD49b+ natural killer cell population proportion of CD45+CD3− lymphocytes (FIG. 14). Moreover, tucatinib treated H2N113 tumors demonstrated a significant decrease in neutrophils (CD11c-F4/80-CD11b+Ly6G+Ly6C+) suppressive MHC II low macrophages, and granulocytic MDSC as proportion of CD45.2+ lymphocytes, and a significant increase in MHC II+ macrophages, CD103+ dendritic cells. Assessment of expression of PD-L1 on total TILs showed an increased intensity of PD-L1 expression on CD45+ cells in tucatinib (T) treated TILs compared with vehicle (V) treated TILs (FIG. 15A-15B).

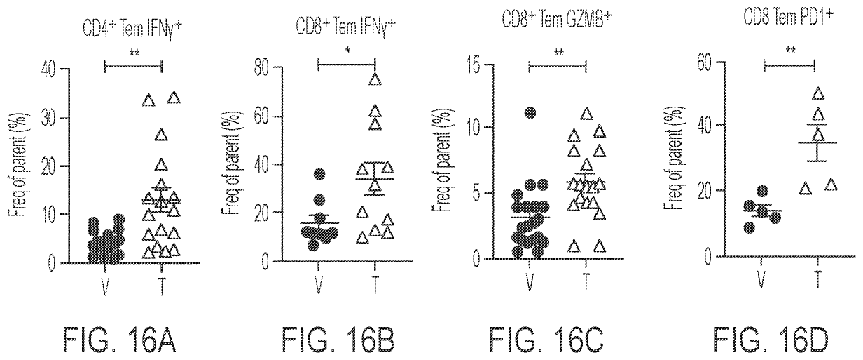

Similar to the H2N113 results of the preceding paragraph, in trastuzumab resistant Fo5 murine tumors treated after 10 days of tucatinib, ex vivo FACS analysis showed increased interferon-7 expression in both CD4+ and CD8+ effector memory T cell subsets (FIG. 16A-16B). CD8+ effector memory T cells also demonstrated increased granzyme B and PD-1+ expression (FIG. 16C-16D). In summary, tucatinib increased frequency and numbers of CD8+ and CD4+ effector cells as well as both effector function and PD-1+ immune checkpoint expression in both trastuzumab sensitive (H2N113) and trastuzumab resistant (Fo5) HER2+ murine tumor models.

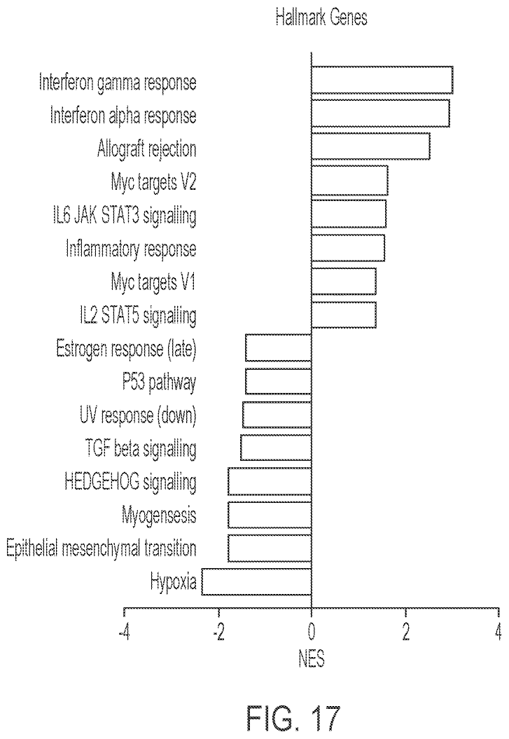

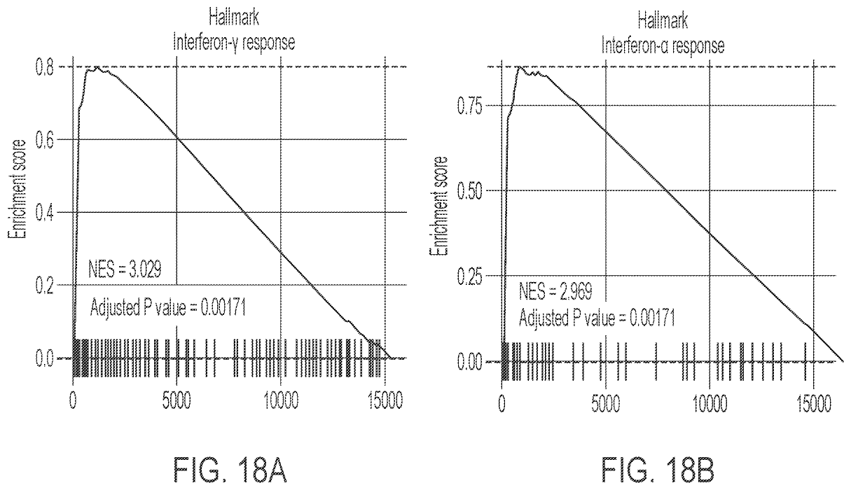

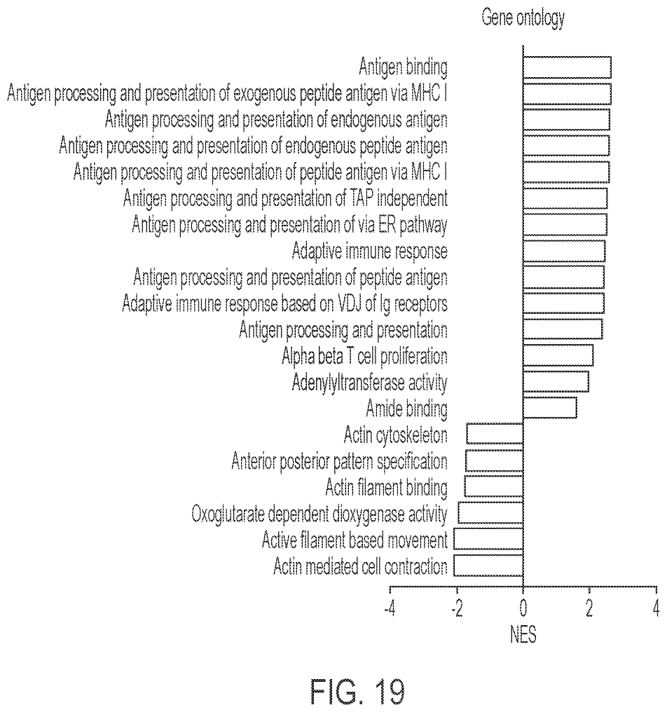

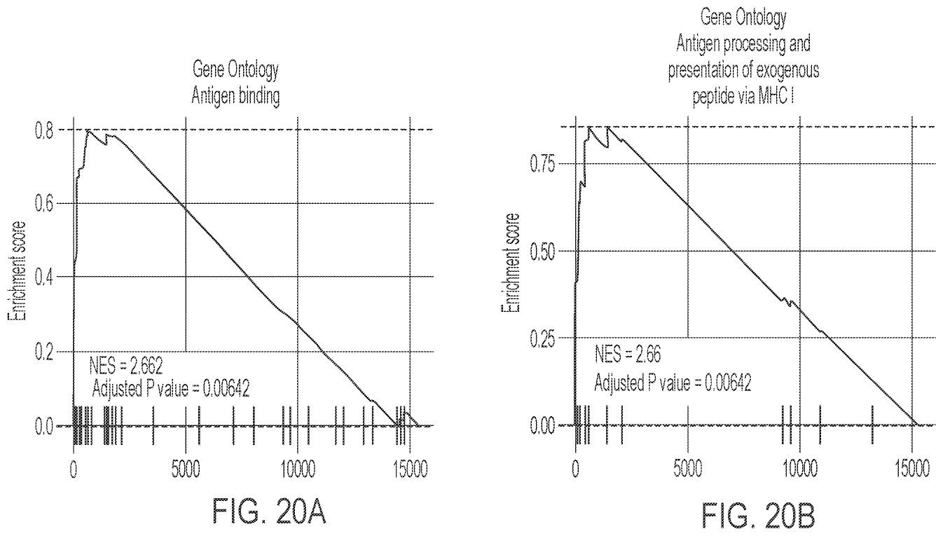

To further understand the effect of tucatinib on the tumor microenvironment in vivo, an unbiased gene expression analysis on Fo5 trastuzumab-resistant tumors treated with tucatinib was performed. The time point for tumor analysis was day 10 post treatment, as determined by FACS, at which point it was observed that effector memory T cell function was significantly different between vehicle and tucatinib treatment groups. A significant upregulation in genes related to immune cell function (FIG. 17 and FIG. 18A-18B) and antigen presentation (FIG. 19 and FIG. 20A-20B) was observed in tucatinib treated tumors compared with vehicle controls (NES=Normalized Enrichment Score). Furthermore, differential gene expression showed upregulation in genes associated with favorable clinical response to checkpoint inhibitors (FIG. 21A-21D) in tucatinib treated tumors. These gene sets are validated from clinical trials and are associated with response to atezolizumab (FIG. 21A) and pembrolizumab (FIG. 21B and FIG. 21C). Within these gene sets, specific genes demonstrating high differential expression include immune related genes such as: CD8A, CD3D, CD3E; genes related to cytokine production: IFNG, GZMB, GZMA, CXCL9, CXCL10, CXCL13, CXCR6, STAT1, TBX21, IDOL; those related to immune checkpoints: CD274, PDCD1LG2, TIGIT; and genes related to T cell and NK cytotoxicity: PRF1, NKG7 (FIG. 21A-21D). These data suggest that tucatinib may increase immunogenicity of the TME by virtue of enhanced antigen presentation and cytokine production in a trastuzumab-resistant model.

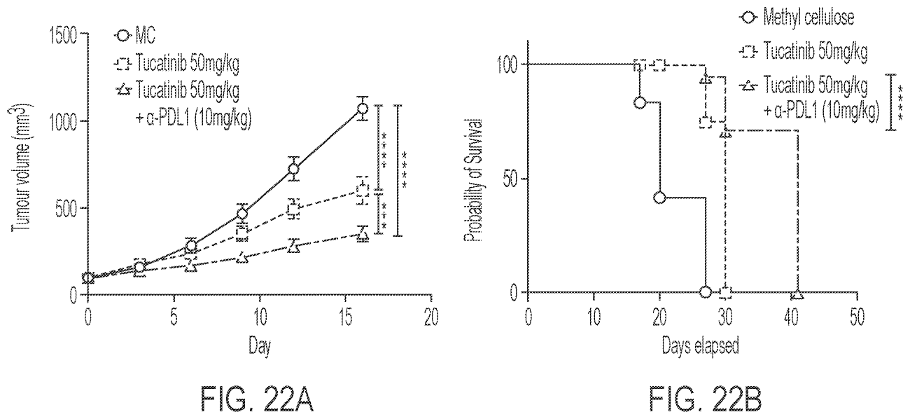

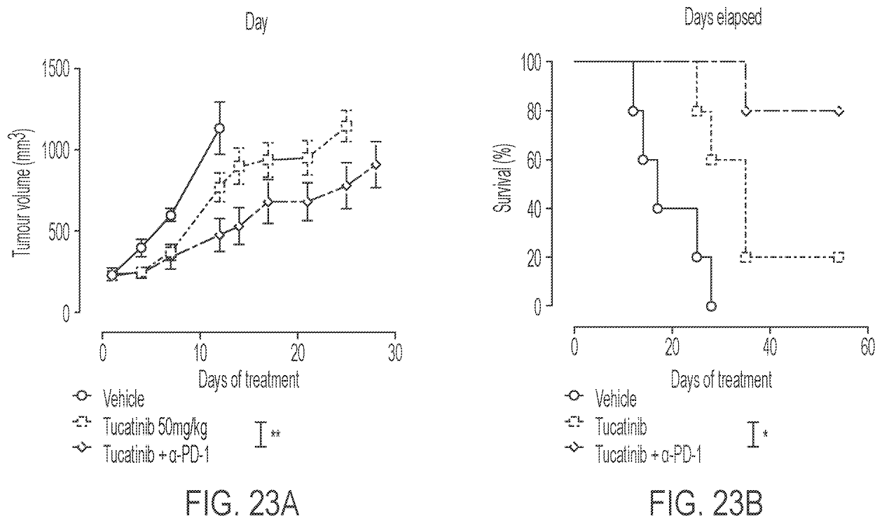

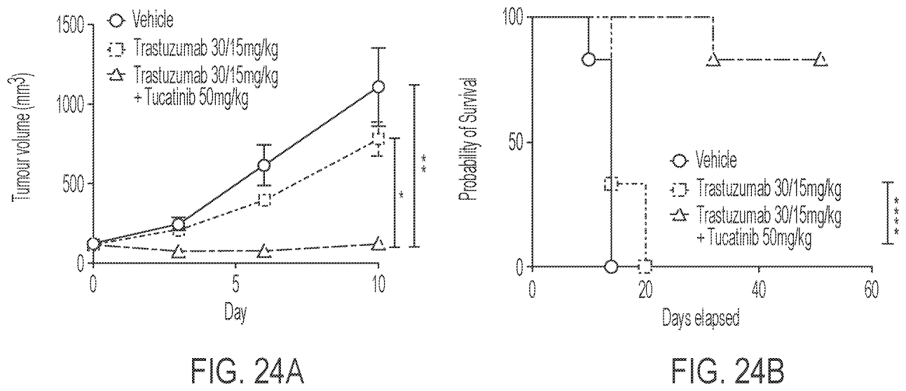

Because IFN-γ expression increases the expression of PD-1/PD-L1, tucatinib in combination with immune checkpoint inhibition in vivo was evaluated. It was found that a PD-L1 inhibitor in combination with tucatinib in mice bearing trastuzumab sensitive H2N113 tumors demonstrated significantly enhanced tumor growth inhibition compared with tucatinib alone and significantly improved survival in the combination treatment group (FIG. 22A-22B). Similarly, in mice with trastuzumab-resistant Fo5 tumors, tucatinib in combination with anti-PD-1 inhibitor was also found to have significantly enhanced anti-tumor effects and significantly better survival than that of tucatinib treatment alone (FIG. 23A-23B). The combination of tucatinib and trastuzumab in the Fo5 model was also investigated and demonstrated that tucatinib overcame the tumor's innate resistance to trastuzumab and resulted in potent anti-tumor efficacy and significantly improved survival (FIG. 24A-24B). These data suggest that the combination of a PD-1 or PD-L1 checkpoint inhibitor and tucatinib could result in improved outcomes for patients with HER2-positive breast cancer. Clinically, this is particularly relevant for those with trastuzumab resistant HER2-positive disease.

1. A method of treating cancer in a subject, the method comprising administering to the subject an antibody or an antigen-binding fragment thereof, wherein the antibody binds to Programmed Death-1 (PD-1) and inhibits PD-1 activity, and tucatinib, or salt or solvate thereof, to the subject, wherein the cancer is a solid tumor.

2. The method of claim 1, wherein the anti-PD-1 antibody or antigen-binding fragment thereof comprises the complementary determining regions (CDRs) of an antibody or antigen-binding fragment selected from the group consisting of pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof.

3. The method of claim 1, wherein the anti-PD-1 antibody or antigen-binding fragment thereof comprises the complementary determining regions (CDRs) of an antibody or antigen-binding fragment of pembrolizumab.

4. The method of claim 1, wherein the anti-PD-1 antibody or antigen-binding fragment thereof comprises the complementary determining regions (CDRs) of an antibody or antigen-binding fragment of nivolumab.

5. The method of claim 1, wherein the anti-PD-1 antibody or antigen-binding fragment thereof comprises the heavy chain variable region and the light chain variable region of an antibody or antigen-binding fragment selected from the group consisting of pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof.

6. The method of claim 1, wherein the anti-PD-1 antibody or antigen-binding fragment thereof comprises the heavy chain variable region and the light chain variable region of an antibody or antigen-binding fragment selected from the group consisting of pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.

7. The method of claim 1, wherein the anti-PD-1 antibody or antigen-binding fragment thereof is selected from the group consisting of pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof.

8. The method of claim 1, wherein the anti-PD-1 antibody or antigen-binding fragment thereof is selected from the group consisting of pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.

9. The method of claim 1, wherein the anti-PD-1 antibody or antigen-binding fragment thereof is pembrolizumab.

10. The method of claim 1, wherein the anti-PD-1 antibody or antigen-binding fragment thereof is nivolumab.

11. The method of any one of claims 1-10, wherein the anti-PD-1 antibody or antigen-binding fragment thereof is administered intravenously.

12. The method of any one of claims 1-11, wherein one or more therapeutic effects in the subject is improved after administration of the tucatinib, or salt or solvate thereof, and the anti-PD-1 antibody or antigen-binding fragment thereof relative to a baseline.

13. The method of claim 12, wherein the one or more therapeutic effects is selected from the group consisting of: size of a tumor derived from the cancer, objective response rate, duration of response, time to response, progression free survival, and overall survival.

14. The method of any one of claims 1-13, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the tucatinib, or salt or solvate thereof, and the anti-PD-1 antibody or antigen-binding fragment thereof.

15. The method of any one of claims 1-14, wherein the objective response rate is at least about 20%, at least about 25%, at least about 30%, at least about 35%, at least about 40%, at least about 45%, at least about 50%, at least about 60%, at least about 70%, or at least about 80%.

16. The method of any one of claims 1-15, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the tucatinib, or salt or solvate thereof, and the anti-PD-1 antibody or antigen-binding fragment thereof.

17. The method of any one of claims 1-16, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the tucatinib, or salt or solvate thereof, and the anti-PD-1 antibody or antigen-binding fragment thereof.

18. The method of any one of claims 1-16, wherein the duration of response to the tucatinib, or salt or solvate thereof, and the anti-PD-1 antibody or antigen-binding fragment thereof is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the tucatinib, or salt or solvate thereof, and the anti-PD-1 antibody or antigen-binding fragment thereof.

19. A method of treating cancer in a subject, the method comprising administering to the subject an antibody or an antigen-binding fragment thereof, wherein the antibody binds to Programmed Death Ligand-1 (PD-L1) and inhibits PD-L1 activity, and tucatinib, or salt or solvate thereof, to the subject, wherein the cancer is a solid tumor.

20. The method of claim 19, wherein the anti-PD-L1 antibody or antigen-binding fragment thereof comprises the complementary determining regions (CDRs) of an antibody or antigen-binding fragment selected from the group consisting of atezolizumab, BMS-936559, durvalumab, avelumab, envafolimab, CK-301, CS-1001, SHR-1316, CBT-502, and BGB-A333, or a biosimilar thereof.

21. The method of claim 19, wherein the anti-PD-L1 antibody or antigen-binding fragment thereof comprises the complementary determining regions (CDRs) of an antibody or antigen-binding fragment of atezolizumab.

22. The method of claim 19, wherein the anti-PD-L1 antibody or antigen-binding fragment thereof comprises the complementary determining regions (CDRs) of an antibody or antigen-binding fragment of BMS936559.

23. The method of claim 19, wherein the anti-PD-L1 antibody or antigen-binding fragment thereof comprises the complementary determining regions (CDRs) of an antibody or antigen-binding fragment of durvalumab.

24. The method of claim 19, wherein the anti-PD-L1 antibody or antigen-binding fragment thereof comprises the complementary determining regions (CDRs) of an antibody or antigen-binding fragment of avelumab.

25. The method of claim 19, wherein the anti-PD-L1 antibody or antigen-binding fragment thereof comprises the heavy chain variable region and the light chain variable region of an antibody or antigen-binding fragment selected from the group consisting of atezolizumab, BMS-936559, durvalumab, avelumab, envafolimab, CK-301, CS-1001, SHR-1316, CBT-502, and BGB-A333, or a biosimilar thereof.

26. The method of claim 19, wherein the anti-PD-L1 antibody or antigen-binding fragment thereof comprises the heavy chain variable region and the light chain variable region of an antibody or antigen-binding fragment selected from the group consisting of atezolizumab, BMS-936559, durvalumab, avelumab, envafolimab, CK-301, CS-1001, SHR-1316, CBT-502, and BGB-A333.

27. The method of claim 19, wherein the anti-PD-L1 antibody or antigen-binding fragment thereof is selected from the group consisting of atezolizumab, BMS-936559, durvalumab, avelumab, envafolimab, CK-301, CS-1001, SHR-1316, CBT-502, and BGB-A333, or a biosimilar thereof.

28. The method of claim 19, wherein the anti-PD-L1 antibody or antigen-binding fragment thereof is selected from the group consisting of atezolizumab, BMS-936559, durvalumab, avelumab, envafolimab, CK-301, CS-1001, SHR-1316, CBT-502, and BGB-A333.

29. The method of claim 19, wherein the anti-PD-L1 antibody or antigen-binding fragment thereof is atezolizumab.

30. The method of claim 19, wherein the anti-PD-L1 antibody or antigen-binding fragment thereof is BMS-936559.

31. The method of claim 19, wherein the anti-PD-L1 antibody or antigen-binding fragment thereof is durvalumab.

32. The method of claim 19, wherein the anti-PD-L1 antibody or antigen-binding fragment thereof is avelumab.

33. The method of any one of claims 19-32, wherein the anti-PD-L1 antibody or antigen-binding fragment thereof is administered intravenously.

34. The method of any one of claims 19-32, wherein one or more therapeutic effects in the subject is improved after administration of the tucatinib, or salt or solvate thereof, and the anti-PD-L1 antibody or antigen-binding fragment thereof relative to a baseline.

35. The method of claim 34, wherein the one or more therapeutic effects is selected from the group consisting of: size of a tumor derived from the cancer, objective response rate, duration of response, time to response, progression free survival, and overall survival.

36. The method of any one of claims 19-35, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the tucatinib, or salt or solvate thereof, and the anti-PD-L1 antibody or antigen-binding fragment thereof.

37. The method of any one of claims 19-36, wherein the objective response rate is at least about 20%, at least about 25%, at least about 30%, at least about 35%, at least about 40%, at least about 45%, at least about 50%, at least about 60%, at least about 70%, or at least about 80%.

38. The method of any one of claims 19-37, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the tucatinib, or salt or solvate thereof, and the anti-PD-L1 antibody or antigen-binding fragment thereof.

39. The method of any one of claims 19-37, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the tucatinib, or salt or solvate thereof, and the anti-PD-L1 antibody or antigen-binding fragment thereof.

40. The method of any one of claims 19-39, wherein the duration of response to the tucatinib, or salt or solvate thereof, and the anti-PD-L1 antibody or antigen-binding fragment thereof is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the tucatinib, or salt or solvate thereof, and the anti-PD-L1 antibody or antigen-binding fragment thereof.

41. The method of any one of claims 1-40, wherein infiltration of natural killer (NK) cells is increased in the solid tumor following administration of tucatinib, or salt or solvate thereof.

42. The method of any one of claims 1-41, wherein infiltration of CD8+ T cells expressing PD-1 is increased in the solid tumor following administration of tucatinib, or salt or solvate thereof.

43. The method of any one of claims 1-42, wherein infiltration of CD8+ T cells expressing IFNγ is increased in the solid tumor following administration of tucatinib, or salt or solvate thereof.

44. The method of any one of claims 1-43, wherein infiltration of CD8+ T cells expressing TIM3 is increased in the solid tumor following administration of tucatinib, or salt or solvate thereof.

45. The method of any one of claims 1-44, wherein infiltration of CD8+ T cells expressing OX40 is increased in the solid tumor following administration of tucatinib, or salt or solvate thereof.

46. The method of any one of claims 1-45, wherein infiltration of CD4+ T cells expressing FOXP3 is increased in the solid tumor following administration of tucatinib, or salt or solvate thereof.

47. The method of any one of claims 1-46, wherein infiltration of CD4+ T cells not expressing FOXP3 is increased in the solid tumor following administration of tucatinib, or salt or solvate thereof.

48. The method of any one of claims 1-47, wherein infiltration of CD4+ T cells expressing Ki67 is increased in the solid tumor following administration of tucatinib, or salt or solvate thereof.

49. The method of any one of claims 1-48, wherein the ratio of CD4+ to CD8+ T cells is increased in the solid tumor following administration of tucatinib, or salt or solvate thereof.

50. The method of any one of claims 1-49, wherein infiltration of neutrophils is decreased in the solid tumor following administration of tucatinib, or salt or solvate thereof.

51. The method of any one of claims 1-50, wherein the percentage of CD11b dendritic cells is increased in the solid tumor following administration of tucatinib, or salt or solvate thereof.

52. The method of any one of claims 1-51, wherein the percentage of MHC-II high expressing macrophages is increased in the solid tumor following administration of tucatinib, or salt or solvate thereof.

53. The method of any one of claims 1-52, wherein the percentage of low expressing macrophages is decreased in the solid tumor following administration of tucatinib, or salt or solvate thereof.

54. The method of any one of claims 1-53, wherein the solid tumor is a HER2+ solid tumor.

55. The method of any one of claims 1-54, wherein the cancer has been determined to express a mutant form of HER2.

56. The method of any one of claims 1-55, wherein the cancer expresses a mutant form of HER2.

57. The method of claim 55 or claim 56, wherein the mutant form of HER2 is determined by DNA sequencing.

58. The method of claim 55 or claim 56, wherein the mutant form of HER2 is determined by determining RNA sequencing.

59. The method of any one of claims 55-58, wherein the mutant form of HER2 is determined by nucleic acid sequencing.

60. The method of claim 59, wherein the nucleic acid sequencing is next-generation sequencing (NGS).

61. The method of claim 55 or claim 56, wherein the mutant form of HER2 is determined by polymerase chain reaction (PCR).

62. The method of any one of claims 55-61, wherein the mutant form of HER2 is determined by analyzing a sample obtained from the subject.

63. The method of claim 62, wherein the sample obtained from the subject is a cell-free plasma sample.

64. The method of claim 62, wherein the sample obtained from the subject is a tumor biopsy.

65. The method of any one of claims 55-64, wherein the HER2 mutation comprises at least one amino acid substitution, insertion, or deletion compared to the amino acid sequence of SEQ ID NO:1.

66. The method of claim 65, wherein the HER2 mutation is an activating mutation.

67. The method of claim 65 or claim 66, wherein the HER2 mutation is a mutation in the extracellular domain, the kinase domain, or the transmembrane/juxtamembrane domain, or any combination thereof.

68. The method of claim 67, wherein the HER2 mutation is a mutation in the extracellular domain selected from the group consisting of G309A, G309E, S310F, S310Y, C311R, C311S, and C334S.

69. The method of claim 67, wherein the HER2 mutation is a mutation in the kinase domain at an amino acid residue selected from the group consisting of Y772, G776, G778, and T798.

70. The method of claim 67, wherein the HER2 mutation is a G776 YVMA insertion.

71. The method of claim 67, wherein the HER2 mutation is a mutation in the kinase domain selected from the group consisting of T733I, L755P, L755S, I767M, L768S, D769N, D769Y, D769H, V777L, V777M, L841V, V842I, N857S, T862A, L869R, H878Y, and R896C.

72. The method of claim 67, wherein the HER2 mutation is a mutation in the kinase domain at an amino acid residue V697.

73. The method of claim 67, wherein the HER2 mutation is a mutation in the transmembrane/juxtamembrane domain selected from the group consisting of S653C, I655V, V659E, G660D, and R678Q.

74. The method of any one of claims 55-73, wherein the cancer does not have HER2 amplification, and wherein the absence of HER2 amplification is determined by immunohistochemistry (IHC).

75. The method of any one of claims 55-73, wherein the cancer has a HER2 amplification score of 0 or 1+, and wherein the HER2 amplification score is determined by immunohistochemistry (IHC).

76. The method of any one of claims 55-75, wherein the cancer has less than a 2 fold increase in HER2 protein levels.

77. The method of any one of claims 1-73, wherein the solid tumor the solid tumor has been determined to comprise HER2 overexpression/amplification.

78. The method of any one of claims 1-73, wherein the solid tumor comprises HER2 overexpression/amplification.

79. The method of claim 77 or claim 78, wherein the HER2 overexpression is 3+ overexpression as determined by immunohistochemistry (IHC).

80. The method of claim 77 or claim 78, wherein the HER2 amplification is determined by an in situ hybridization assay.

81. The method of claim 80, wherein the in situ hybridization assay is fluorescence in situ hybridization (FISH) assay.

82. The method of claim 80, wherein the in situ hybridization assay is chromogenic in situ hybridization.

83. The method of claim 77 or claim 78, wherein the HER2 amplification is determined in tissue by NGS.

84. The method of claim 77 or claim 78, wherein the HER2 amplification is determined in circulating tumor DNA (ctDNA) by a blood-based NGS assay.

85. The method of any one of claims 1-84, wherein the solid tumor is a metastatic solid tumor.

86. The method of any one of claims 1-85, wherein the solid tumor is locally-advanced.

87. The method of any one of claims 1-86, wherein the solid tumor is unresetable.

88. The method of any one of claims 1-87, wherein the solid tumor is selected from the group consisting of cervical cancer, uterine cancer, gallbladder cancer, cholangiocarcinoma, urothelial cancer, lung cancer, breast cancer, gastroesophageal cancer, and colorectal cancer.

89. The method of claim 88, wherein the breast cancer is a HER2+ breast cancer.

90. The method of claim 88 or claim 89, wherein the breast cancer is hormone receptor positive (HR+) breast cancer.

91. The method of claim 88, wherein the lung cancer is non-small cell lung cancer.

92. The method of any one of claims 1-91, wherein the solid tumor is sensitive to trastuzumab.

93. The method of any one of claims 1-91, wherein the solid tumor is resistant to trastuzumab.

94. The method of any one of claims 1-93, wherein the tucatinib, or salt or solvate thereof, is administered to the subject at a dose of about 150 mg to about 650 mg.

95. The method of claim 94, wherein the tucatinib, or salt or solvate thereof, is administered to the subject at a dose of about 300 mg.

96. The method of claim 94 or 95, wherein the tucatinib, or salt or solvate thereof, is administered once or twice per day.

97. The method of claim 96, wherein the tucatinib, or salt or solvate thereof, is administered to the subject at a dose of about 300 mg twice per day.

98. The method of any one of claims 1-97, wherein the tucatinib, or salt or solvate thereof, is administered to the subject orally.

99. The method of any one of claims 1-98, further comprising administering one or more additional therapeutic agents to the subject to treat the cancer.

100. The method of claim 99, wherein the one or more additional therapeutic agents is an anti-CTLA4 antibody or antigen-binding fragment thereof.

101. The method of claim 100, wherein the anti-CTLA4 antibody or antigen-binding fragment thereof comprises the CDRs of ipilimumab, or a biosimilar thereof.

102. The method of claim 100, wherein the anti-CTLA4 antibody or antigen-binding fragment thereof comprises the heavy chain variable region and the light chain variable region of ipilimumab, or a biosimilar thereof.

103. The method of claim 100, wherein the anti-CTLA4 antibody or antigen-binding fragment thereof is ipilimumab, or a biosimilar thereof.

104. The method of any one of claims 100-103, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express CTLA4.

105. The method of any one of claims 1-104, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1.

106. The method of any one of claims 1-105, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1.

107. The method of any one of claims 1-106, wherein treating the subject results in a tumor growth inhibition (TGI) index of at least about 85%.

108. The method of any one of claims 1-107, wherein treating the subject results in a TGI index of about 100%.

109. The method of any one of claims 1-108, wherein the subject has one or more adverse events and is further administered an additional therapeutic agent to eliminate or reduce the severity of the one or more adverse events.

110. The method of any one of claims 1-109, wherein the subject is at risk of developing one or more adverse events and is further administered an additional therapeutic agent to prevent or reduce the severity of the one or more adverse events.

111. The method of claim 109 or claim 110, wherein the one or more adverse events is a grade 3 or greater adverse event.

112. The method of claim 109 or claim 110, wherein the one or more adverse events is a serious adverse event.

113. The method of any one of claims 1-112, wherein the subject is a human.

114. The method of any one of claims 1-113, wherein the tucatinib, or salt or solvate thereof, is in a pharmaceutical composition comprising the tucatinib, or salt or solvate thereof, and a pharmaceutical acceptable carrier.

115. The method of any one of claims 1-18, wherein the anti-PD-1 antibody or antigen-binding fragment thereof is in a pharmaceutical composition comprising the anti-PD-1 antibody or antigen-binding fragment thereof and a pharmaceutical acceptable carrier.

116. The method of any one of claims 19-40, wherein the anti-PD-L1 antibody or antigen-binding fragment thereof is in a pharmaceutical composition comprising the anti-PD-L1 antibody or antigen-binding fragment thereof and a pharmaceutical acceptable carrier.

117. The method of any one of claims 100-104, wherein the anti-CTLA4 antibody or antigen-binding fragment thereof is in a pharmaceutical composition comprising the anti-CTLA4 antibody or antigen-binding fragment thereof and a pharmaceutical acceptable carrier.

118. A kit comprising:
(a) tucatinib, or salt or solvate thereof;
(b) an antibody or an antigen-binding fragment thereof, wherein the antibody binds to Programmed Death-1 (PD-1) and inhibits PD-1 activity; and
(c) instructions for use of the tucatinib, or salt or solvate thereof and the anti-PD-1 antibody or antigen-binding fragment thereof according to the method of any one of claims 1-18.

119. A kit comprising:
(a) tucatinib, or salt or solvate thereof;
(b) an antibody or an antigen-binding fragment thereof, wherein the antibody binds to Programmed Death Ligand-1 (PD-L1) and inhibits PD-L1 activity; and
(c) instructions for use of the tucatinib, or salt or solvate thereof and the anti-PD-L1 antibody or antigen-binding fragment thereof according to the method of any one of claims 19-40.
